(19) United States
(12) Patent Application Publication
(10) Pub. No.: US 2026/0327606 A1
(43) Pub. Date: Oct. 22, 2026

(54) ANTIBODY SCREENING

(71) Applicant: The Johns Hopkins University, Baltimore, MD (US)

(72) Inventors: Bert Vogelstein, Baltimore, MD (US); Kenneth W. Kinzler, Frankford, DE (US); Nickolas Papadopoulos, Towson, MD (US); Shibin Zhou, Owings Mills, MD (US); Steve Lu, Baltimore, MD (US); Austin Mattox, Baltimore, MD (US)

(21) Appl. No.: 19/107,510

(22) PCT Filed: Aug. 30, 2023
(86) PCT No.: PCT/US2023/031476

§ 371 Date: Feb. 27, 2025

Related U.S. Application Data
(60) Provisional application No. 63/402,827, filed on Aug. 31, 2022.

(51) Int. Cl. C12N 15/10; C12Q 1/6804; C40B 30/04; G01N 33/68

(52) U.S. Cl. CPC C12N 15/1068; C12Q 1/6804; G01N 33/6857; C40B 30/04; G01N 2500/04

(57) ABSTRACT

Described herein are, among other things, compositions and methods for antibody screening.

### CROSS-REFERENCE TO RELATED APPLICATIONS

This application claims priority to U.S. Provisional Patent Application No. 63/402,827, filed on Aug. 31, 2022, which is incorporated herein by reference in its entirety.

### FEDERALLY SPONSORED RESEARCH OR DEVELOPMENT

This invention was made with government support under grants CA006973 and GM136577 awarded by the National Institutes of Health. The government has certain rights in the invention.

### SEQUENCE LISTING

This application contains a Sequence Listing that has been submitted electronically as an XML file named “44807-0415WO1_ST26_SL.XML.” The XML file, created on Aug. 29, 2023, is 424,065 bytes in size. The material in the XML file is hereby incorporated by reference in its entirety.

### TECHNICAL FIELD

Described herein are, among other things, compositions and methods for antibody screening.

### BACKGROUND

In the past two decades, three zoonotic beta-coronaviruses have crossed species and infected humans with high mortality and impact on society. Most recently during the coronavirus disease 2019 (COVID-19) pandemic, the novel severe acute respiratory syndrome-related coronavirus 2 (SARS-COV-2) has infected over 260 million people with numbers still rising as of November 2021.3 Despite the remarkable efforts to develop therapies for infected individuals, the persistence of the pandemic as well as the potential for future outbreaks highlight limitations of current approaches to identify antibody-based therapies against a highly transmissible and rapidly evolving virus. This is just one of many examples of the need for rapid and comprehensive approaches to develop high quality antibodies for therapeutic applications.

### SUMMARY

With the sudden emergence of SARS-COV-2 viruses, the need to quickly develop antibody-based therapeutics has been highlighted.^{1,2} Described herein is an approach, called SLISY (Sequencing-Linked ImmunoSorbent assay) that has the potential to rapidly identify antibodies to any antigen, including new viral variants. As one example, SLISY was able to recover 1279 clones that specifically bound to the Receptor Binding Domain region of SARS-COV-2 but not that of MERS. Eighty-eight percent of these 1279 clones bound to at least one of six different receptor-binding domain (RBD) variants of SARS-COV-2, and 114 clones bound to all six RBDs. By identifying clones that bound to multiple variants simultaneously, clones were also recovered that exhibit broad-spectrum neutralizing potential against each of the variants tested. As will be appreciated by those of ordinary skill in the art, composition and methods described herein are broadly applicable to the rapid identification of antibodies, or antigen binding fragment thereof, that bind any antigen. For example, compositions and methods described herein are useful in the rapid identification of antibodies, or antigen binding fragment thereof, that bind any antigen of therapeutic interest including those associated with infectious disease (e.g., SARS-COV-2, influenza, Ebola, HIV, Dengue, and Middle Eastern Respiratory Syndrome (MERS), or any other viral or non-viral human disease caused by pathogens), cancer, autoimmune diseases, and the like. SLISY can be used to assess specificity of binding of millions of clones simultaneously, can in principle be applied to any screen that links DNA sequence to a potential binding moiety (e.g., phage, yeast, or ribosome display libraries) and requires only a single round of biopanning.

Provided herein are methods comprising: (i) providing a display library comprising a plurality of library members, wherein each library member comprises a display protein and a nucleic acid encoding the display protein, wherein each display protein comprises a complementarity determining region (CDR) H3 region; (ii) performing a binding assay of the display library against a target antigen to enrich for target-bound display proteins, and isolating nucleic acid from the target bound library members to generate a target bound nucleic acid sample; (iii) performing a binding assay of the display library against an off-target antigen to enrich for off-target-bound display proteins and isolating nucleic acid from the off-target bound library members to generate an off-target bound nucleic acid sample; (iv) determining the number of nucleic acid molecules comprising one or more of the CDR H3 regions of the display proteins of the display library in a representative sample of each of the target-bound nucleic acid sample and the off-target bound nucleic acid sample; (v) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one.

In some embodiments, the methods can further comprise: (vi) comparing the ratio of to a reference value; (vii) if the ratio is larger than the reference value, determining the sequence of the CDR H1, CDR H2, CDR H3, CDR L1, CDR L2, and CDR L3 sequences of one or more of the display proteins of the display library; and (viii) optionally, performing a binding assay on the one or more display proteins from step (vii) against the on-target antigen and/or the off-target antigen.

Also provided herein are methods for identifying an antibody, or antigen binding fragment thereof, that binds to a target antigen, the method comprising: (i) providing a display library comprising a plurality of library members, wherein each library member comprises a display protein and a nucleic acid encoding the display protein, wherein each display protein comprises a complementarity determining region (CDR) H3 region; (ii) performing one or more rounds of selection on the display library against a first target antigen to generate a selected display library; (iii) performing a binding assay of the selected display library against a target antigen to enrich for target-bound display proteins, and isolating nucleic acid from the target bound library members to generate a target bound nucleic acid sample; (iv) performing a binding assay of the display library against an off-target antigen to enrich for off-target-bound display proteins and isolating nucleic acid from the off-target bound library members to generate an off-target bound nucleic acid sample; (v) determining the number of nucleic acid molecules comprising one or more of the CDR H3 regions of the display proteins of the display library in a representative sample of each of the target-bound nucleic acid sample and the off-target bound nucleic acid sample; and (vi) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one.

In some embodiments, the methods can further comprise: (vii) comparing the ratio of to a reference value; (viii) if the ratio is larger than the reference value, determining the sequence of the CDR H1, CDR H2, CDR H3, CDR L1, CDR L2, and CDR L3 sequences of one or more of the display proteins of the display library, thereby identifying an antibody or antigen binding fragment thereof that binds to a target antigen; and (ix) optionally, performing a binding assay on the one or more display proteins from step (viii) against the on-target antigen and/or the off-target antigen.

In some embodiments, the number of library members in the display library is at least 102, at least 103, at least 104, at least 105, at least 106, at least 107, at least 108, or at least 109.

In some embodiments, determining the number of nucleic acid molecules comprises massively parallel sequencing. In some embodiments, massively parallel sequencing comprises sequencing-by-synthesis. In some embodiments, determining the number of nucleic acid molecules comprises PCR amplification using primers that universally amplify the CDR H3 region of the display proteins and also incorporate a molecular barcode.

In some embodiments, the one or more rounds of selection comprises one or more rounds comprising positive selection against the target antigen; and/or one or more rounds comprising negative selection against an off-target antigen; and, optionally, amplification. In some embodiments, the method comprising a first round of selection, wherein the first round of selection comprises or consists of positive selection against the target antigen. In some embodiments, the first round of selection comprises or consists of positive selection against the target antigen followed by negative selection against the off-target antigen.

In some embodiments, the method comprises between two and ten rounds of selection, wherein each of the second through tenth rounds of selection comprises or consists of positive selection against a target antigen and/or negative selection against the off-target antigen to generate a selected display library, and wherein the input display library at the start of each round of selection is the selected display library output from the last round of selection.

In some embodiments, positive selection against the target antigen comprises positive selection against a cell line expressing a target antigen and negative selection comprises selection against a cell line not expressing the target antigen. In some embodiments, the cell line not expressing the target antigen is a genetic knockout or knockdown version of the cell line expressing the target antigen. In some embodiments, positive selection against the target antigen comprises positive selection against a substrate comprising the target antigen and negative selection comprises negative selection against the substrate not comprising the target antigen.

In some embodiments, the display library is a phage display library. In some embodiments, the phage display library is an M13 phage display library. In some embodiments, the display proteins comprise scFvs. In some embodiments, the display proteins consist of a fusion protein comprising an scFv. In some embodiments, the fusion protein comprises or consists of a phage coat protein or portion thereof and an scFv.

In some embodiments, the reference value is 1, 2, 3, 4, 5, 6, 7, 8, 9, 10, 11, 12, 13, 14, 15, 16, 17, 18, 19, or 20.

Also provided herein are antigens or antigen binding fragments thereof identified by any one of the methods described herein.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a schematic of an example of the Sequencing-Linked ImmunoSorbent assay (SLISY). In this example, a sample of scFv phage library is applied to the desired target as well as an appropriate negative control. After extensive washing, bound phage are eluted, followed by PCR amplification and sequencing of the CDR-H3 region with universal primers to determine SLISY Binding Ratios (SBR) for each particular phage clone. SBR is calculated by comparing the number of phage eluted from the target well/cells with identical CDR-H3 sequence to the phage eluted from the non-target (negative) well/cells with the same CDR-H3 sequence. In this example, CDR-H3 sequences that have a molecular count of zero are given a value of 0.5 to avoid division by zero.

FIG. 1B shows low representation of clones with high SBR. Fraction of each clone within the entire pool after four rounds of panning against the RBD is plotted against its SBR. Red box indicates clones that are likely good binders (high SBR) but at low abundance.

FIG. 1C shows enrichment of clones across multiple rounds of biopanning. The top 100 ranked clones were determined based on their SBR values at round 4. The red line represents average enrichment of the round 4 top 100 clones; the grey line represents average enrichment of remaining clones.

FIGS. 2A-2E show validation of highly specific SARS-COV-2 clones. FIG. 2A shows SBRs for each clone is plotted against its PER from baseline after four rounds of biopanning. Red clones represent those selected for validation. Note one clone, RBD_6, was highly enriched in all three biopanning strategies but only selected as an RBD candidate as indicated. FIG. 2B shows binding specificity of candidate SARS-COV-2 scFvs. Wells pre-coated with RBD, S1 or FL spike proteins from SARS-COV-2 or MERS were incubated with phage. “—CTRL”, a negative control using phage expressing scFv against HLA-A3. Error bars represent standard deviation of means (n=3). C. Neutralizing potential of SARS-COV-2 specific phage tested using FDA approved cPASS assay. Phage (1013 titer) or antibodies pre-mixed with HRP-RBD were applied to wells pre-coated with recombinant human ACE2 protein. Red region represents ≥30% neutralization compared to HRP-RBD alone. “MM42”, a non-neutralizing negative control mAb; “MM57”, a neutralizing positive control mAb; “+CTRL”, a manufacturer-provided positive control. FIG. 2D shows correlation of SBR between round 4 and round 1 of RBD panning using 100×deeper sequencing. Clones in red represent those that were in the top 100 for both round 1 and round 4. Clones in blue represent those that were in top 100 of round 4 but not round 1. Clones in yellow represent those that were in top 100 of round 1 but not round 4. FIG. 2E shows performance of clones selected after one round of biopanning. Clones in the top 100 of round 1 after deep sequencing were assessed for their final outcomes after four rounds of biopanning.

FIGS. 3A-3D show characterization of converted full-length SARS-COV-2 antibodies. FIG. 3A is a schematic showing the relationship between scFv-expressing phage and full-length antibodies. FIG. 3B shows specificity of full length antibodies grafted from validated scFv-expressing phage. Wells pre-coated with RBD, S1 or FL spike proteins from SARS-COV-2 or MERS were incubated with converted antibodies. A3_13, which binds to HLA-A3, serves as a negative control for the ELISA. FIG. 3C shows measuring neutralization with cPASS assay. Red shaded region represents ≥30% neutralization and is defined as detection of SARS-COV-2 neutralizing activities. “MM42”, a non-neutralizing negative control mAb. “MM57”, a neutralizing positive control mAb. “+CTRL”, a manufacturer provided positive control. FIG. 3D shows the ability of full-length antibodies to inhibit ACE2-spike protein interaction. The converted full-length antibodies were applied to wells pre-coated with RBD, S1 or FL spike proteins from SARS-COV-2, followed by the addition of recombinant ACE2-His protein. Error bars represent standard deviation of means (n=3).

FIGS. 4A-4E show an example of using SLISY to assess binding and identify clones across multiple variants simultaneously. FIG. 4A shows assessing clones across multiple RBD variants using SLISY. UpSet plot includes upright bars that represent the total number of cross-reactive clones in each intersection. Filled circles below bars represent the variants that represent the intersecting set. Intersecting sets that have fewer than 6 cross-reactive clones were not represented. Horizontal bars represent the total number of clones that had a SLISY Ratio of ten and above for each of the proteins. FIG. 4B shows SBR for selected clones across variants. FIG. 4C shows ELISA for selected clones across variants. FIG. 4D shows selecting variant binding clones for validation. After applying the enriched pool of scFvs from biopanning with the original SARS-COV-2 to variant proteins, the original SARSCOV-2 SBR for each clone was plotted against a variant SBR. Clones were selected for testing based on high SBRs for both the original and variants. Twenty clones were selected from each of the original-variant pairings. FIG. 4E shows broad spectrum neutralization by “superclone” scFvs Beta_10, Gamma_12, and Gamma 19. The full-length scFv phage clones were applied to wells pre-coated with variant SARS-COV-2 spike proteins, followed by the addition of recombinant ACE2-His protein. Negative control is A3-Clone 13 scFv phage. Error bars represent standard deviation of means (n=3).

FIG. 5 shows Primer design for long-read sequencing. Initial amplification of template was done using forward and reverse primers flanking the entire scFv sequence. Amplified template was then used for long-read sequencing on an Illumina Sequencer. Read 1 primer was used to sequence CDRL-1, -2, and -3 regions as well as the molecular barcodes. A custom index primer was used to sequence the highly diverse CDRH-3 as well as the sample index. Read 2 primer was used to sequence the CDRH-1 and CDRH-2 regions.

FIG. 6 shows Overview of panning strategy for phage clones against HLA-A3. The scFv phage display library was expanded and applied to CFPAC parental (HLA-A2 & HLA-A3 positive) cells for positive biopanning and CFPAC HLA-A3 knockout (KO) cells for negative biopanning against HLA-A2 and other cell surface proteins. After washing and pelleting cells from positive biopanning, phage were eluted and used to infect SS320 bacteria with helper phage for amplification and expression of the scFv. After overnight growth, phage were precipitated for PEG/NaCl. After 4 rounds of biopanning, enriched phage were applied to both cell lines for the SLISY assay and analysis.

FIG. 7 shows Selection of HLA-A3 clones by cell-based SLISY. SBR versus Panning Enrichment Ratio (PER) for all clones (circles) in HLA-A3 biopanning, where PER for each clone was calculated by dividing its fraction in the population after growth by the fraction present in the input library and converting it to log base 2. SBR and PER were plotted after four rounds of biopanning. Eleven clones (red) were selected for subsequent validation and had an SBR greater than 10. The clone in yellow represents A3-Clone 20 to indicate its performance relative to other clones after it was spiked-in at a final ratio of 1:105 phage. A3-Clone 20 is an A3-specific clone previously identified using traditional panning and served as a positive control for A3 specificity. Consistent with our expectations, the CDR-H3 representative of A3-Clone 20 yielded an SBR significantly greater than 1 (7.02+0.42, mean±SD, n=3) in the sample derived from the spike-in library. More importantly, 62 novel CDR-H3 clones with SBRs greater than that observed for A3-Clone 20 were observed in either the nascent library or the library containing spiked in A3-Clone 20 (Extended Data Table 1). This apparent superior binding was often not reflected by the enrichment observed in the more traditional PER based scoring. PER and SBR were calculated as described in the Materials and Methods. The A3-Clone 20 was only detected in the spike-in library and only five of the high scoring scFvs overlapped between the two biopannings, suggesting the wide diversity of clones available in the library for HLA binding and supporting our concerns about the stochastic nature of traditional growth-based panning. Finally, while these novel CDR-H3 sequences could be readily identified with SLISY, only 1 of 62 were present at greater than 0.2% (1 in 500) after four rounds of biopanning, making it unlikely that they would be successfully identified by random sampling of phage from the final pool of growth selected phage.

FIGS. 8A-8B show Validation of HLA A3-specific candidate scFv clones. Using the long-read sequencing, we derived the full sequence of 11 scFvs showing high SBRs (>10) for HLA-A3 and used them to create synthetic gene blocks for the expression of the scFvs as clonal phagemids. FIG. 8A: Binding of candidate clones to biotinylated HLA-A3 versus HLA-A2. A3-Clone 20 served as a positive control for HLA-A3 specificity. All 11 monoclonal scFvs displayed preferential HLA-A3 protein binding relative to HLA-A2 protein in a standard ELISA assay. FIG. 8B: Binding of clones to CFPAC parental cells that express HLA-A3 versus isogenic HLA-A3 KO cell line. “—CTRL” represents the background binding of secondary antibodies without any initial phage. All 11 scFv monoclonals showed preferential binding of HLA-A3 expressing cells versus their isogenic HLA-A3 knockout by flow cytometry.

FIG. 9 shows a scheme of the SARS-COV-2 FL monomer (amino acids 16-1273). The N-terminal domain (NTD, blue) comprises amino acids 16-303; the core receptor binding domain (RBD, orange) comprises amino acids 319-541. The NTD and RBD form the S1 subunit. The S2 subunit (green) comprises amino acids 685-1213.

FIG. 10 shows a schematic of biopanning and SLISY for SARS-COV-2 specific scFvs. Sequences of candidate clones identified for SARS-COV-2 targets with high SBR are determined by NGS of the entire scFv as described in FIG. 5. The scFv sequences are used to generate geneblocks which are then cloned into the phagemid vector.

FIGS. 11A-11B show Distribution of SARS-COV-2 specific clones after biopanning. FIG. 11A: Overlap of SARS-COV-2 candidate clones from the biopannings of RBD, S1, and FL spike protein. Binding of clones along the spike protein after four rounds of biopanning was detected by SLISY. Only clones that had SBR greater than 10 were considered. 516 unique clones were independently selected and identified in all three biopannings. FIG. 11B: Localization of clones selected around FL spike protein. To determine the relative regions where the clones bound on SARS-COV-2 spike protein, the polyclonal phage from four rounds of FL biopanning was applied to the RBD, S1, and FL spike proteins. Independent SBRs determined using SLISY. Of the clones that were selected using the FL spike protein, 32% bound to the RBD region.

FIGS. 12A-12H show Selected SARS-COV-2 mAbs. FIGS. 12A-12F. Gel filtration chromatograms of S1_13, FL_5, FL_10, FL_12, FL_13, and RBD_1 mAbs, respectively; mAbs eluted at ~12 mL or ~18 mL with a 20 mM Na Phosphate, pH 7.2, 150 mM NaCl buffer. Chromatograms are representative of three replicates. FIG. 12G: Sodium dodecyl-sulfate polyacrylamide gel electrophoresis (SDS-PAGE) of the mAbs in A-F. The lanes in order are MW marker, and, in consecutive lanes reducing (R) and non-reducing (NR) conditions for the S1_13, FL_5, FL_10, FL_12, FL_13, and RBD_1 mAbs, respectively. In the R lanes, bands at 50 and 25 kDa represent the heavy and light chains, respectively; in the NR lanes, the band at ~150 kDa represents the full-length mAb. FIG. 12G: Affinity of SLISY selected-mAbs to SARS-COV-2 protein as measured by SPR. RBD_1 mAb binding to SARS-COV-2 FL spike protein was assessed with single-cycle kinetics using SPR (red line). The RBD_1 mAb was attached to a protein A chip and SARS-COV-2 FL spike protein was loaded at increasing concentrations (5 nM, 20 nM, 80 nM, 160 nM, 320 nM). RBD_1 mAb bound to the SARS-COV-2 FL spike protein with a KD of 44.2 nM when fit to one-to-one binding kinetics (grey line). The blank- and reference subtracted binding curve is shown. Other studies carried out in the same way as RBD_1 but for the following selected mAbs: FL_10 (KD of 7.5 nM), S1_13 (KD of 11.7 nM), FL_12 (KD of 6.0 nM), FL_5 (KD of 39.7 nM), FL_13 (KD of 9.5 nM) respectively.

FIG. 13 shows Epitope binning of SARS-COV-2 antibodies. The column indicates the biotinylated mAbs, and the row indicates the competing non-biotinylated mAbs. An indirect ELISA format was used to identify whether isolated neutralizing antibody clones competed for the same antigenic epitope on the SARS-COV-2 RBD polypeptide. Biotinylated antibody is mixed with each free antibody with the pre-titrated concentration through a volume ratio of 1:1 and applied to an indirect ELISA format to analyze competition within each pair. Inhibition of 70% or greater indicates strong simultaneous binding between the two antibodies likely due to same overlapping epitopes (dark blue squares). Inhibition of 50-70% indicates partial binding (light blue squares), while inhibition less than 50% suggests no simultaneous binding (white squares). Self-competition of an antibody against itself are represented by yellow squares.

FIGS. 14A-14B show Assessing clones across multiple SARS-COV-2 variants using SLISY. FIG. 14A: S1 variants. FIG. 14B: FL variants. UpSet plots include upright bars that represent the total number of cross-reactive clones in each intersection. Filled circles below bar represent the variants that represent the intersecting set. Horizontal bars represent the total number of clones that had a SBR of ten and above for each of the proteins. Error bars represent standard deviation of means (n=3).

FIG. 15 shows specificity of full-length SARS-COV-2 Antibody Clones for Multiple Variants. Wells pre-coated with spike protein were incubated with antibodies followed by anti-human IgG Fc-HRP antibody before being developed. A3_13, which binds to HLA-A3, serves as a phage negative control for the ELISA.

FIG. 16 shows neutralization potential of full-length SARS-COV-2 antibody clones for multiple variants. Wells coated with spike protein antigens were incubated with converted antibodies for 1 hr. After vigorous washing, wells were then incubated with recombinant hACE2-His protein, followed by rabbit anti-His polyclonal antibody, and finally goat anti-rabbit IgG antibody HRP for 1 hr at each step.

FIG. 17 shows binding activity of Beta, Gamma, and Delta selected scFvs.

FIG. 18 shows neutralization activity of Beta, Gamma, and Delta selected scFvs.

FIG. 19 shows ability of full-length antibodies to block pseudovirus infectivity.

FIG. 20 shows ability of full-length antibodies to block variant pseudovirus infectivity.

### DETAILED DESCRIPTION

Antibody-based therapies have been shown to provide immediate passive protection and to complement prophylactic vaccines. A notable example of this principle is convalescent plasma, though limited by scalability.⁴ Another example is ansuvimab (mAb114), which provided a safe and effective treatment against symptomatic Ebola virus disease.^{5,6} Other examples are provided by monoclonal antibodies against HIV, Dengue, and Middle Eastern Respiratory Syndrome (MERS). Such antibodies have been identified in screens using Epstein-Barr virus-immortalized memory B cells.⁷⁻¹⁰ These highly effective approaches are, however, limited by the time required to obtain samples from recovered patients as well as the time required for identification and isolation of specific, high affinity antibodies.¹¹

In vitro selection using display technologies, such as biopanning with libraries made in phage, bacteria, yeast, mammalian cells, or with ribosomes, enable particularly efficient ways to select for highly specific binding elements.^{12,13} Furthermore, promising candidates can be amplified and enriched through repeated rounds of positive selection with the target.¹⁴⁻¹⁶ Another advantage over conventional antibody generation is that display technologies allow for negative biopanning, which reduce clones that may be cross-reactive with closely related target antigens.

Traditional display library screens employing Sanger sequencing limit the number of clones to be screened to 10²-10³. Advances in next-generation sequencing (NGS) have allowed for deeper insights into the diversity by providing up to 10⁷ sequences (10,000-fold more).¹⁷⁻¹⁹ Although the strength of all of these methods relies on a direct link between phenotype and genotype, high throughput methods to assess clones suffer from imperfections in this link. Specifically, selection for high antigen-affinity clones through multiple rounds of biopanning can be confounded by unpredictable, growth-based library biases as well as stochastic sampling of clones that are at low frequency in the library. Moreover, up until now, sequencing of the entire length of some clone inserts (i.e. 600 bp for single-chain variable fragments (scFvs)) has not been possible without the isolation, cloning and sequencing of individual candidates, which drastically limits throughput.

Described herein, among other things, is an approach that combines NGS with differential binding assays to simultaneously evaluate all clones in any library even after a single round of screening and rapidly proceed to antibody production.

Thus, described herein are method comprising: (i) providing a display library comprising a plurality of library members, wherein each library member comprises a display protein and a nucleic acid encoding the display protein, wherein each display protein comprises a complementarity determining region (CDR) H3 region; (ii) performing a binding assay of the display library against a target antigen to enrich for target-bound display proteins, and isolating nucleic acid from the target bound library member to generate a target bound nucleic acid sample; (iii) performing a binding assay of the display library against an off-target antigen to enrich for off-target-bound display proteins and isolating nucleic acid from the off-target bound library member to generate an off-target bound nucleic acid sample; (iv) determining the number of nucleic acid molecules comprising one or more of the CDR H3 regions of the display proteins of the display library in a representative sample of each of the target-bound nucleic acid sample and the off-target bound nucleic acid sample; (v) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one.

In some cases, the method further comprises (vi) comparing the ratio of to a reference value; (vii) if the ratio is larger than the reference value, determining the sequence of the CDR H1, CDR H2, CDR H3, CDR L1, CDR L2, and CDR L3 sequences of one or more of the display proteins of the display library; and (viii) optionally, performing a binding assay on the one or more library members from step (vii) against the on-target antigen and/or the off-target antigen.

In some cases, method described herein comprise: (i) providing a display library comprising a plurality of display proteins, each comprising a complementarity determining region (CDR) H3 region; (ii) performing a binding assay of the display library against a target antigen to enrich for target-bound display proteins, and isolating nucleic acid from the target bound display proteins to generate a target bound nucleic acid sample; (iii) performing a binding assay of the display library against an off-target antigen to enrich for off-target-bound display proteins and isolating nucleic acid from the off-target bound display proteins to generate an off-target bound nucleic acid sample; (iv) determining the number of nucleic acid molecules comprising one or more of the CDR H3 regions of the display proteins of the display library in a representative sample of each of the target-bound nucleic acid sample and the off-target bound nucleic acid sample; (v) or each at least one of the one or more CDR H3 sequences, comparing the number of molecules comprising the CDR H3 sequence in the target-bound nucleic acid sample to the number of molecules comprising the CDR H3 sequence in the off-target-bound nucleic acid sample. In some cases, a count of zero in the denominator is rounded to a positive number less than one.

Comparing the number of molecules comprising the CDR H3 sequence in the target-bound nucleic acid sample to the number of molecules comprising the CDR H3 sequence in the off-target-bound nucleic acid sample can be performed using any of a variety of methods. For example, the comparison can be performed by determining a ratio of the number of molecules comprising the CDR H3 sequence in the target-bound nucleic acid sample to the number of molecules comprising the CDR H3 sequence in the off-target-bound nucleic acid sample. In some cases, the comparison can be performed by determining the number of molecules comprising the CDR H3 sequence in the target-bound nucleic acid sample to the number of molecules comprising the CDR H3 sequence in the off-target-bound nucleic acid sample. In some cases, the comparison is performed with normalization. In some cases, the comparison is performed without normalization. In some cases, the comparison can be performed by determining a ratio of the number of molecules comprising the CDR H3 sequence in the target-bound nucleic acid sample to the number of molecules comprising the CDR H3 sequence in the starting unenriched library. In some cases, the comparison can be performed by determining the number of molecules comprising the CDR H3 sequence in the target-bound nucleic acid sample to the number of molecules comprising the CDR H3 sequence in the starting unenriched library. In some cases, the comparison is performed with normalization. In some cases, the comparison is performed without normalization.

In some cases, the comparison can be performed by determining a ratio of the number of molecules comprising the CDR H3 sequence in the target-bound nucleic acid sample of a particular selection round to the number of molecules comprising the CDR H3 sequence in a previous selection round. In some cases, the comparison can be performed by determining the number of molecules comprising the CDR H3 sequence in the target-bound nucleic acid sample of a particular selection round to the number of molecules comprising the CDR H3 sequence in a previous selection round. In some cases, comparison is performed between sequential selection rounds (e.g., comparing the first round to the second round). In some cases, comparison is performed between sequential selection rounds (e.g., comparing the first round to the third or subsequent round). In some cases, comparison is performed between multiple selection rounds (e.g., comparing the first round to the second and the third round). In some cases, the comparison is performed with normalization. In some cases, the comparison is performed without normalization.

In some cases, the comparison can be performed by determining a ratio of the number of molecules comprising the CDR H3 sequence in the target-bound nucleic acid sample to the number of molecules comprising the CDR H3 sequence in a sample from a separate selection in which the display library is selected for binding to a different target. In some cases, the comparison can be performed by determining the number of molecules comprising the CDR H3 sequence in the target-bound nucleic acid sample to the number of molecules comprising the CDR H3 sequence in a sample from a separate selection in which the display library is selected for binding to a different target. In some cases, the different target is a target that is related to the intended target. For example, the intended target can be an antigen that is specific to a given pathogen, and the different target can be a closely related antigen that is specific to a different pathogen. In some case, the intended target can be an antigen that is specific to a given pathogen, and the different target can be a mutated version of that antigen that differs by one or more amino acid residues. In some cases, the comparison is performed with normalization. In some cases, the comparison is performed without normalization.

Also described herein are methods for identifying an antibody, or antigen binding fragment thereof, that binds to a target antigen, the method comprising: (i) providing a display library comprising a plurality of library members, wherein each library member comprises a display protein and a nucleic acid encoding the display protein, wherein each display protein comprises a complementarity determining region (CDR) H3 region; (ii) performing one or more rounds of selection on the display library against a first target antigen to generate a selected display library; (iii) performing a binding assay of the selected display library against a target antigen to enrich for target-bound display proteins, and isolating nucleic acid from the target bound library members to generate a target bound nucleic acid sample; (iv) performing a binding assay of the display library against an off-target antigen to enrich for off-target-bound display proteins and isolating nucleic acid from the off-target bound library members to generate an off-target bound nucleic acid sample; (v) determining the number of nucleic acid molecules comprising one or more of the CDR H3 regions of the display proteins of the display library in a representative sample of each of the target-bound nucleic acid sample and the off-target bound nucleic acid sample; (vi) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one; (vii) comparing the ratio of to a reference value; (viii) if the ratio is larger than the reference value, determining the sequence of the CDR H1, CDR H2, CDR H3, CDR L1, CDR L2, and CDR L3 sequences of one or more of the display proteins of the display library, thereby identifying an antibody or antigen binding fragment thereof that binds to a target antigen; and (ix) optionally, performing a binding assay on the one or more display proteins from step (viii) against the on-target antigen and/or the off-target antigen.

Also provided herein are antibodies, or antigen binding fragments thereof, identified by the methods described herein.

In some cases, the methods described herein utilize display libraries. Display libraries include, for example, phage display libraries, ribosome display libraries, mRNA display libraries, cis-activity based (CIS) display libraries, covalent antibody display libraries, aptamer display libraries, and in vitro compartmentalization display libraries. See, e.g., Galan et al., “Library-based display technologies: where do we stand?” Mol. BioSyst. doi: 10.1039/c6mb00219f (2016).

In some cases, the display library is a phage display library. Phage display libraries are well known in the art and described, for example, in Ledsgaard et al., “Basics of Antibody Phage Display Technology,” Toxin 10(6):236 (2018), Almagro et al., “Phage Display Libraries for Antibody Therapeutic Discovery and Development,” Antibodies 8:44 (2019), and Anand et al., “Phage Display Technique as a Tool for Diagnosis and Antibody Selection for Coronaviruses,” Current Microbiology 78:1124-34 (2021). As is known in the art, a given library member of a phage display library comprises a display protein and a nucleic acid encoding the display protein.

In some cases, the library is an M13 phage display library.

In some cases, the number of library members in the display library is at least 10², at least 10³, at least 10⁴, at least 10⁵, at least 10⁶, at least 10⁷, at least 10⁸, at least 10⁹, at least 10¹⁰, or at least 10¹¹. In some cases the number of library members in the display library is between 10² and 10¹². In some cases, the number of library members in the display library is between 10⁹ and 10¹².

In some cases, the phage display library is a naïve library. In some cases, the phage display library is a synthetic library. In some cases, the phage display library is a semisynthetic library.

In some cases, the display protein comprises an antibody or antigen binding fragment thereof comprising a CDR H3 region (e.g., an scFv).

In some cases the display library comprises fusion proteins, e.g., of phage coat proteins and antibodies or antigen binding fragments thereof comprising a CDR H3 region.

In some cases, the methods described herein comprise binding assays. Such binding assays can be performed before or after selection (e.g., after any round of selection in a method that includes multiple rounds), including both positive and negative selection. Suitable binding assays include, but are not limited to: Enzyme-linked Immunosorbent Assays (ELISAs), Immunosorbent Assays, Immune precipitation, Immunohistochemistry, and Western Blots. Other suitable binding assays are known in the art.

In some cases, the methods described herein comprise molecular counting, e.g., of nucleic acid molecules. Suitable methods for molecular counting include, but are not limited to those that incorporate random nucleotide barcodes of length 4 to 20 as molecular tags such as applied in SafeSeqS or SaferSeqS.

In some cases, molecular counting comprises molecular barcoding and sequencing of the nucleic acid molecules, e.g., as described in Kinde et al., Detection and quantification of rare mutations with massively parallel sequencing, Proc Natl Acad Sci USA. 2011 Jun. 7; 108 (23): 9530-5. doi: 10.1073/pnas.1105422108, Mattox et al., Bisulfite-converted duplexes for the strand-specific detection and quantification of rare mutations, Proc Natl Acad Sci USA. 2017 May 2; 114 (18):4733-4738. doi: 10.1073/pnas. 1701382114, and Cohen et al., Detection of low-frequency DNA variants by targeted sequencing of the Watson and Crick strands, Nat Biotechnol. 2021 October; 39 (10): 1220-1227. doi: 10.1038/s41587-021-00900-z, each of which is incorporated by reference herein in its entirety.

In some cases, determining the number of nucleic acid molecules comprises massively parallel sequencing. In some cases, massively parallel sequencing comprises sequencing-by-synthesis.

In some cases, determining the number of nucleic acid molecules comprises PCR amplification using primers that universally amplify the CDR H3 region of the display proteins and also incorporate a molecular barcode. In some cases, a molecule barcode can be a unique identifier (UID), such as a UID used in “Safe-SeqS” (Safe-Sequencing System) methods, which are described in Kinde et al. Detection and quantification of rare mutations with massively parallel sequencing, Proc Natl Acad Sci USA. 2011 Jun. 7; 108 (23):9530-5. doi: 10.1073/pnas. 1105422108, and PCT application publication no. WO/2012/142213, each of which is incorporated by reference herein in its entirety. Safe-SeqS methods generally include the following steps: i) assignment of a unique identifier (UID) to a population of template molecules; (ii) amplification of uniquely tagged template molecule to create UID-families; and (iii) redundant sequencing of the amplification products. Safe-SeqS methods are useful in determining the number of nucleic acid molecules comprising one or more of the CDR H3 regions in any of the variety of methods described herein.

In some cases, the methods described herein comprise one or more rounds of selection (e.g., positive selection and/or negative selection, e.g., biopanning. Selection strategies are well known in the art and described, for example, in Anand et al., “Phage Display Technique as a Tool for Diagnosis and Antibody Selection for Coronaviruses,” Current Microbiology 78:1124-34 (2021).

In some cases, the one or more rounds of selection comprises one or more rounds comprising positive selection against the target antigen; and/or one or more rounds comprising negative selection against an off-target antigen; and, optionally, amplification.

In some cases, the methods described herein comprise a first round of selection, wherein selection comprises or consists of positive selection against the target antigen.

In some cases, the first round of selection comprises or consists of positive selection against the target antigen followed by negative selection against an off-target antigen.

In some cases, the method comprises between two and ten rounds of selection, wherein each of the second through tenth rounds of selection comprises or consists of positive selection against a target antigen and/or negative selection against a target antigen to generate a selected display library, and wherein the input display library at the start of each round of selection is the selected display library output from the last round of selection.

In some cases, positive selection against the target antigen comprises positive selection against a cell line expressing a target antigen and negative selection comprises selection against a cell line not expressing the target antigen. In some cases, the cell line not expressing the target antigen is a genetic knockout or knockdown version of the cell line expressing the target antigen.

In some cases, positive selection against the target antigen comprises positive selection against a substrate comprising the target antigen and negative selection comprises negative selection against the substrate not comprising the target antigen.

### Examples

The invention is further described in the following examples, which do not limit the scope of the invention described in the claims.

### Example 1: Basic elements of SLISY

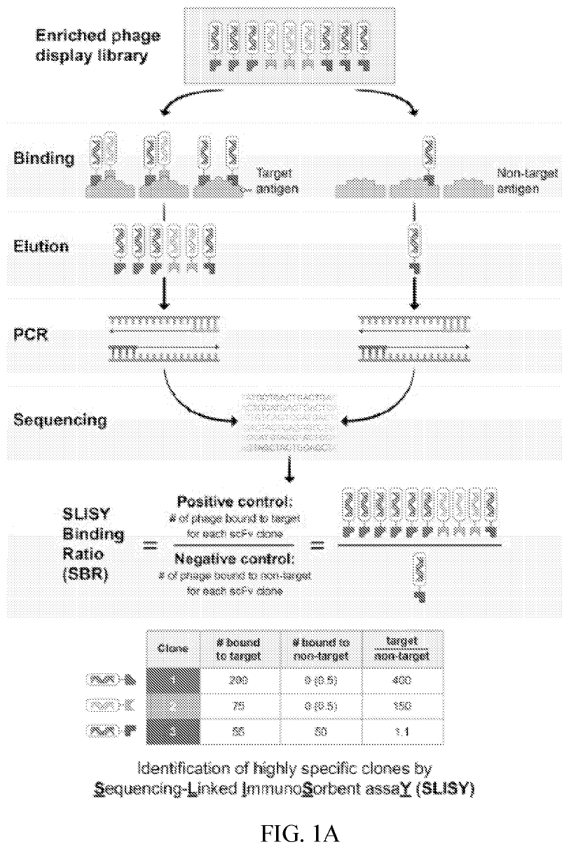

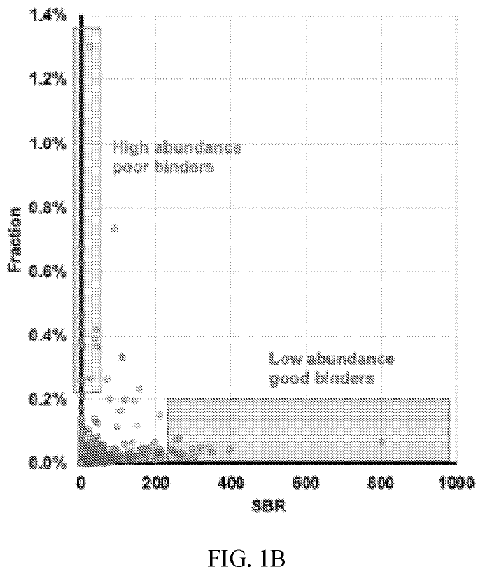

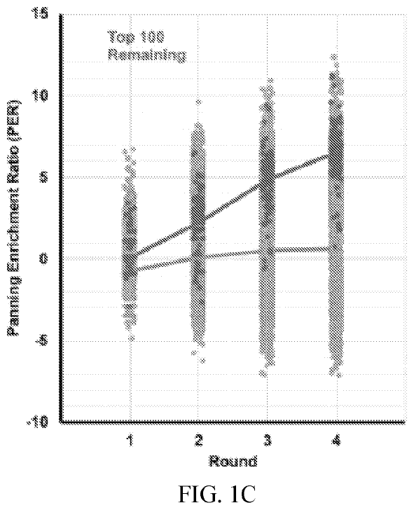

Commonly applied phage display-based approaches for identification of antigen-specific scFvs rely on multiple rounds of biopanning. On the basis of extensive experience with this process, it was postulated that the direct assessment of scFv phage binding without the intermediate growth phases could accelerate and improve the identification of desirable scFvs.²⁰⁻²² To test this hypothesis, a quantitative NGS based phage binding assay called SLISY (Sequencing-Linked ImmunoSorbent assay) was first developed. SLISY is based on using the highly diverse complementarity determining region H3 (CDR-H3) region as a unique identifier of each scFv-expressing phage (FIG. 1A). Primers were designed that could universally amplify the CDR-H3 region of any scFv with high efficiency while also incorporating a unique molecular barcode to avoid introduction of PCR bias (rather than phage growth bias) in subsequent analyses.²³ It was possible to then use NGS to detect and directly enumerate phage binding to a target antigen versus non target or control antigens. The resulting molecular counts could then be used to calculate a SLISY Binding Ratio (SBR) and identify binders as depicted in FIG. 1A. In the second component of our strategy, a NGS sequencing strategy that allowed isolation of the full-length sequence of any phage using only the CDR-H3 region as a key to query a custom en masse sequence library of the input phage (FIG. 5) was developed. These sequences could then be used to rapidly reconstitute binding phage or converted to full-length antibodies. These methods and others related to the fundamentals of SLISY are described in detail in the Materials and Methods section below.

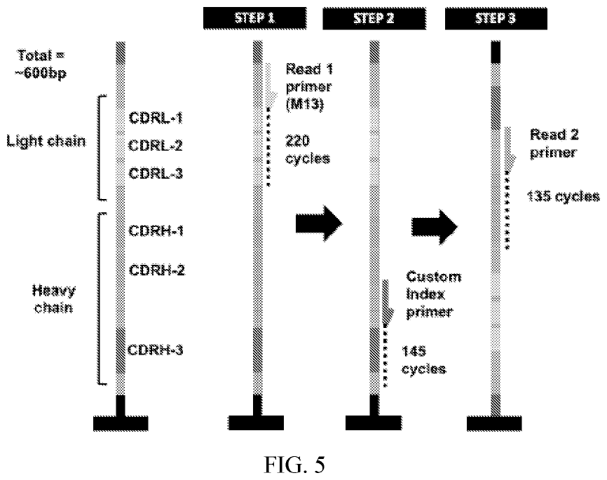

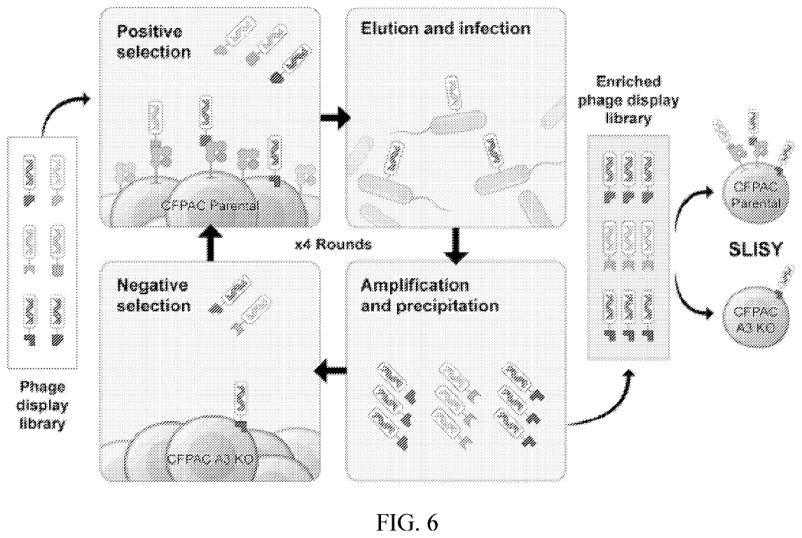

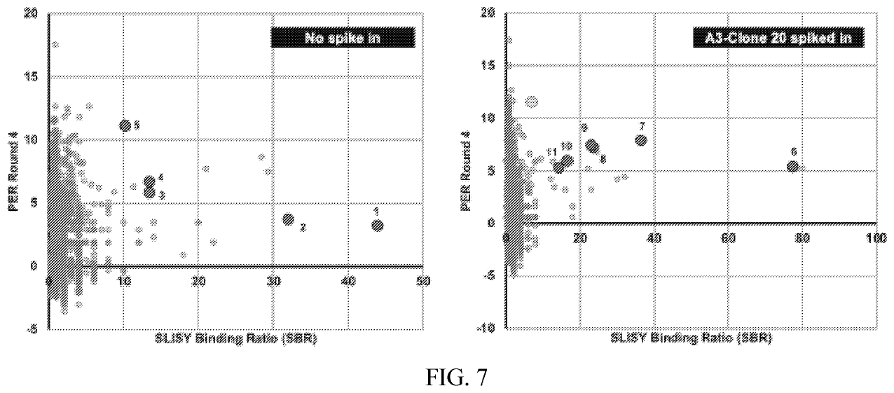

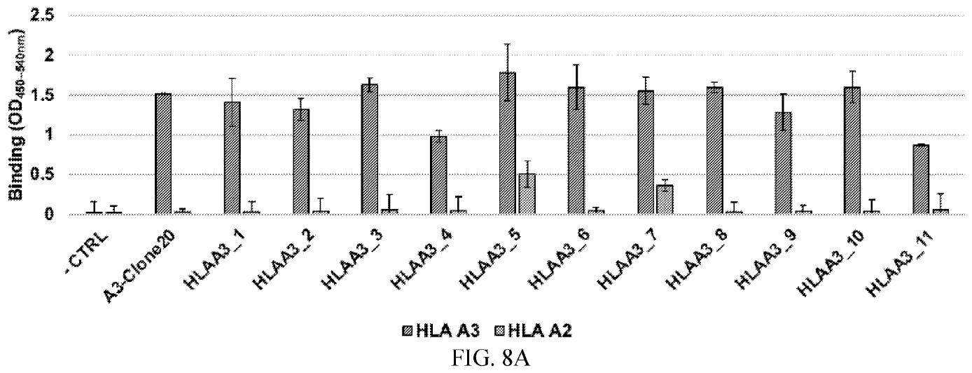

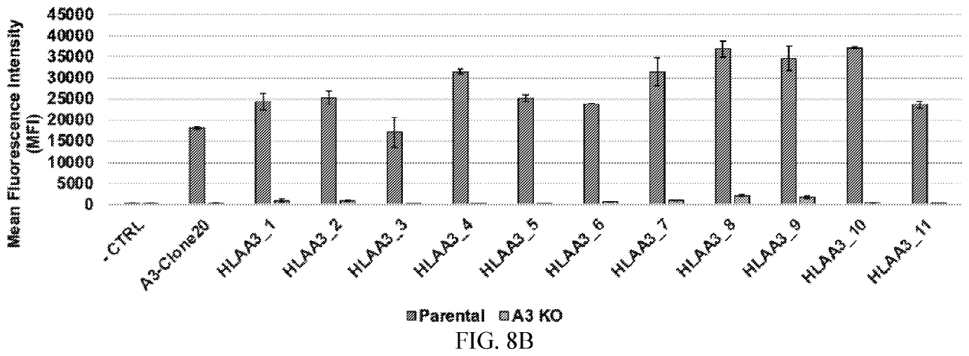

As an initial test of the above strategy, it was sought to identify antibodies that bound specifically to Human Leukocyte Anitgen-A3 (HLA-A3) allele versus HLA-A2. We chose cell-bound HLA as an initial target because of the availability of positive controls and the need to apply negative biopanning to ensure the desired specificity (FIG. 6). These initial experiments indicated that SLISY could identify desirable clones, which would be difficult to identify using a traditional growth based screening approach (FIG. 7 and Table A). Consistent with this, a panel of 11 full-length scFvs displaying HLA-A3 specific binding by enzyme-linked immunosorbent assay (ELISA) and flow cytometry (FIGS. 8A-8B and Table B) was produced. The results of the cell-based screening for HLA-A3 suggested that SLISY could improve both the number and quality of clones derived from biopanning and that the SLISY approach warranted further investigation.

### Example 2: Identification of Highly Specific SARS-COV-2 Antibody Fragments

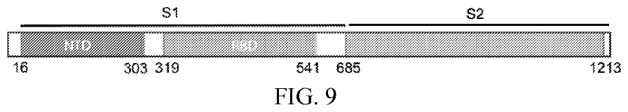

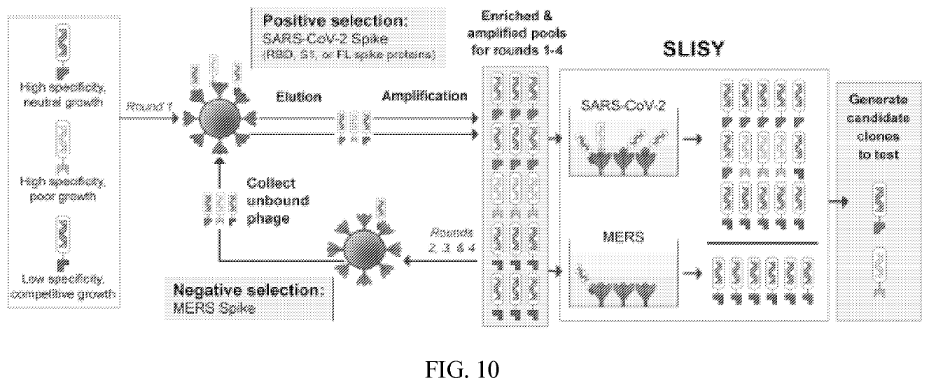

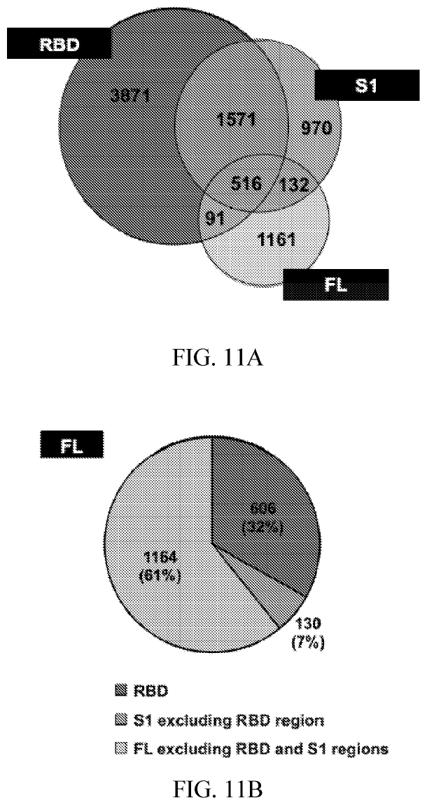

Given the importance of SARS-COV-2 and its emerging variants, it was next tested whether SLISY could rapidly identify antibody fragments that specifically bound to the SARS-COV-2 spike protein. Selection for phage binding to the full-length (FL) SARS-COV-2 spike protein (amino acids 16 to 1213), to its S1 subunit (S1, amino acids 16-685), or to the core receptor binding domain (RBD, amino acids 319-541) (FIGS. 9A-9B) was performed. Four rounds of traditional biopanning were performed against each target with negative selection against the related coronavirus MERS (FIG. 10). After each round, an SBR for every scFv clone was calculated as the observed read count of its CDR-H3 sequence recovered after binding to the SARS-COV-2 polypeptides divided by the observed read count of the same CDR-H3 sequence recovered after binding to the MERS protein. After four rounds of biopanning (see below for results of a single round of biopanning), we observed 8312 unique scFv clones with SBR>10 for the full-length spike protein, S1, or RBD domains (FIG. 11A). Of the subset of clones that were selected for binding to the full-length spike protein, 32% bound to the RBD region of S1 and 7% bound to the non-RBD portion of S1, while the remainder (61%) bound to other regions of the FL protein (FIG. 11B).

As observed with HLA-A3 biopanning, many of the most specific SARS-CoV-2 clones (defined by SLISY) were at very low frequency even after four rounds of enrichment and expansion. Conversely, the great majority of the most abundant clones were not highly specific (FIG. 1B). For example, the clone with the highest SBR was only present in one of 2437 clones (0.04%). If clones were selected for evaluation by conventional methods (i.e., a few hundred clones picked randomly after four rounds), this highly specific clone would have been missed. The top 100 clones as assessed by SLISY only represented 1.1 to 4.0% of the final pools for each of the three targets after four rounds (Table C). To measure enrichment, a Panning Enrichment Ratio (PER) for each clone was calculated by dividing its fraction in the population after growth by the fraction present in the input library and converting it to log base 2. Although only a small final fraction, clones that scored highest by SLISY were continually enriched across each round (FIG. 1C).

### Example 3: Validation of the Specificity of SARS-COV-2 Clones Identified with SLISY

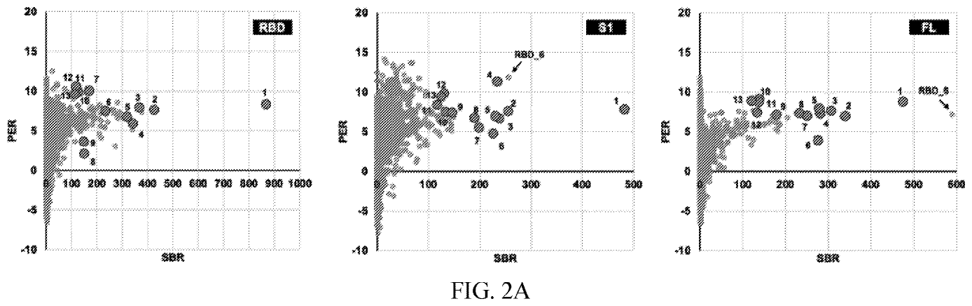

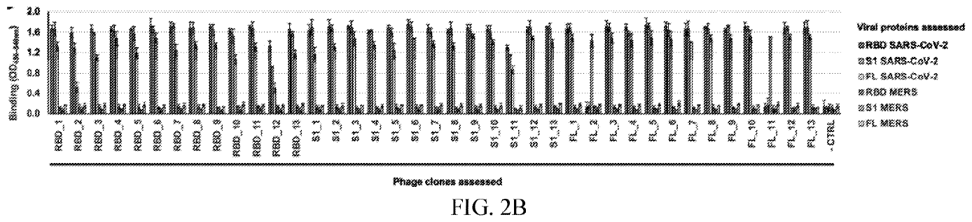

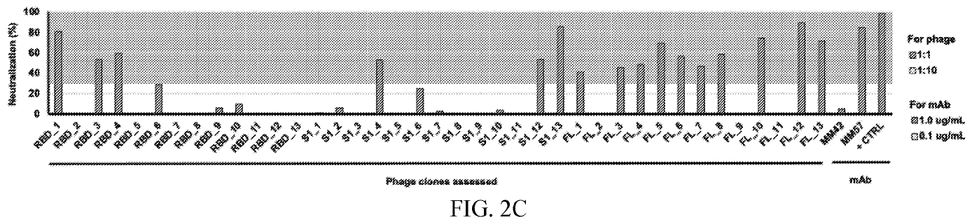

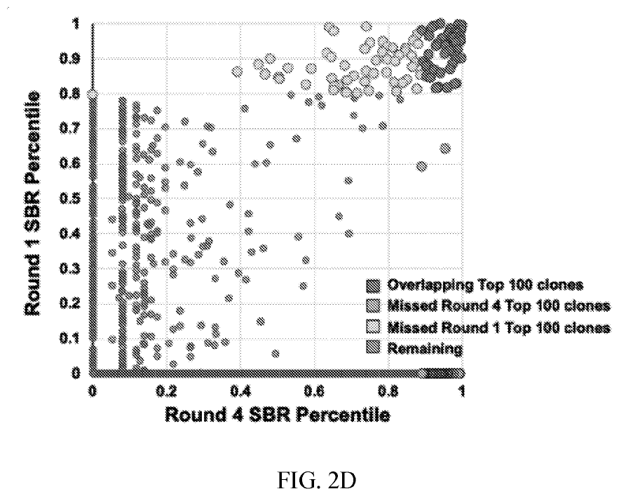

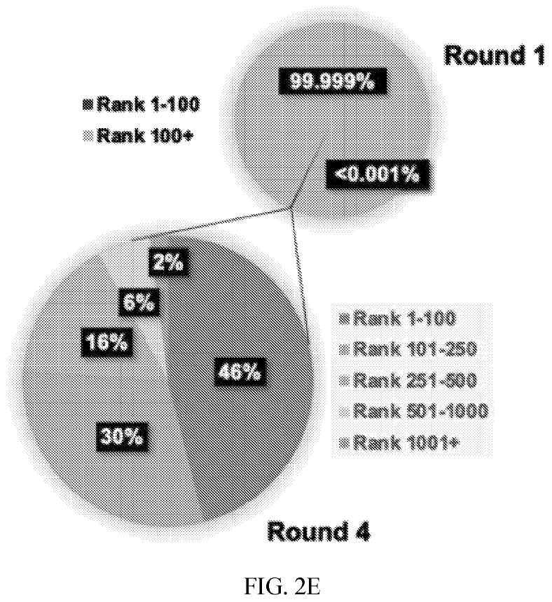

To validate the specificity of the clones identified by SLISY in an orthogonal fashion, candidate scFv-expressing phage were directly generated by sequencing the entire length of scFvs as described above (Table F). For validation, 39 clones were chosen, thirteen from each of the three proteins used for screening with each clone having an SBR>100 (FIG. 2A). One-hundred percent of these 39 clones were found to specifically bind to SARS-COV-2 via ELISA, a standard immunoassay (FIG. 2B). This perfect concordance is not unexpected in that SLISY can be thought of as a type of digital ELISA assay, though it evaluates millions of “antibodies” in parallel rather than the relatively small number that can be evaluated via ELISA.²⁴

To be therapeutically useful, an antibody must neutralize the virus rather than simply bind to the SARS-COV-2 spike protein. Indeed, most antibodies against the SARS-COV-2 spike protein are not neutralizing because they do not bind to the region required to interact with angiotensin converting enzyme 2 (ACE2), the cellular receptor for the virus.²⁵ An FDA-approved test (cPASS) was used to evaluate the neutralizing capacity of the 39 phage clones described above. Over 40% of clones were found to be neutralizing, often as strongly neutralizing as commercial antibodies (FIG. 2C).

### Example 4: SLISY Allows Identification of Clones after a Single Round of Biopanning

Although four rounds of biopanning were employed for the experiments described above, it was suspected that functional clones could be obtained after one round of biopanning. Moreover, it was believed that additional rounds might be unnecessary and decrease the diversity of identified clones by virtue of growth bias: highly specific clones might grow less well than other clones during phage expansion, which is a known problem with biopanning.²⁶ The most definitive of experiments to address this issue was a comparison of the SLISY data after one versus four rounds of biopanning. It was found that 46% of the 100 most specific clones in Round 4 were found among the 100 most specific clones in Round 1 (FIG. 2D). Conversely, 76% of the of 100 most specific clones in Round 1 were found within the 250 most specific clones in Round 4 (FIG. 2E). Importantly, these data suggest that neither sequential positive section nor negative selection is required during the biopanning process when SLISY is employed; biopanning against MERS proteins was not used for Round 1 and only used for Rounds 2, 3, and 4. This makes the entire biopanning process considerably simpler, quicker and allows identification of clones specific for multiple antigens in a single step, as subsequent experiments confirmed.

### Example 5: Full-Length Monoclonal Antibodies (mAbs) Against SARS-COV-2 Derived from Phage Clones

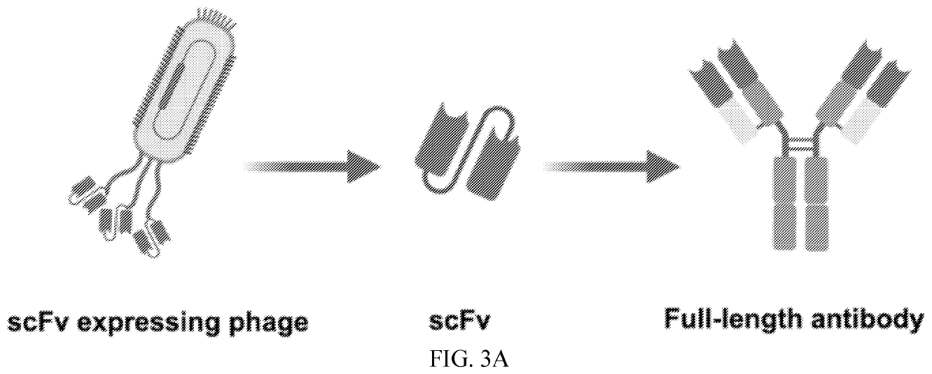

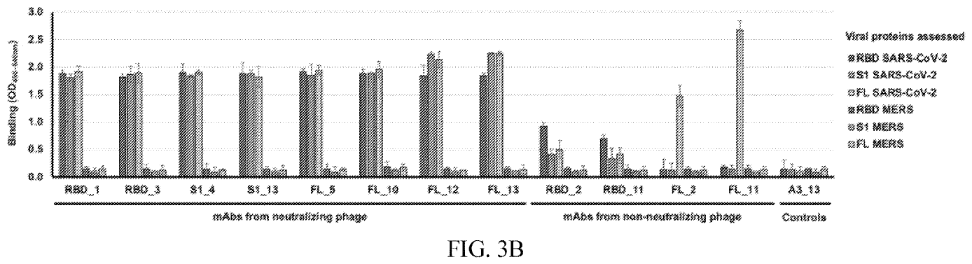

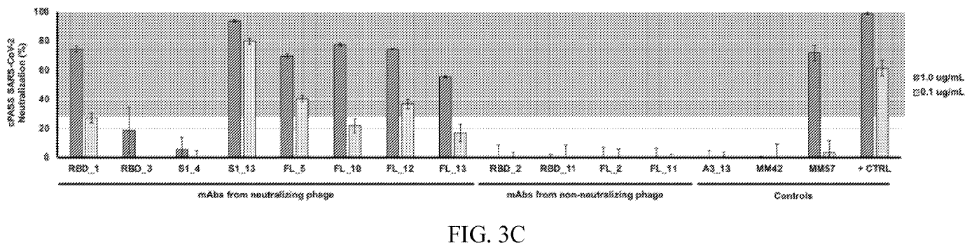

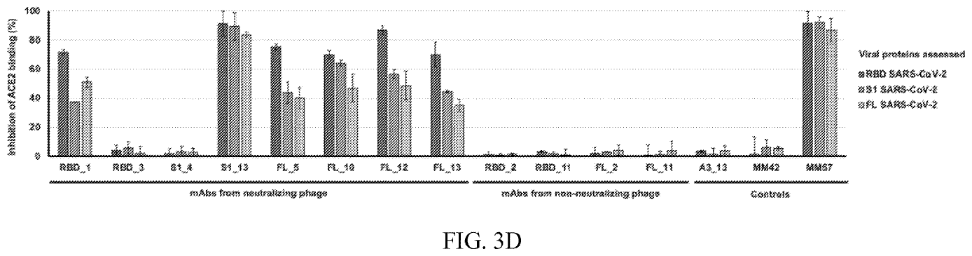

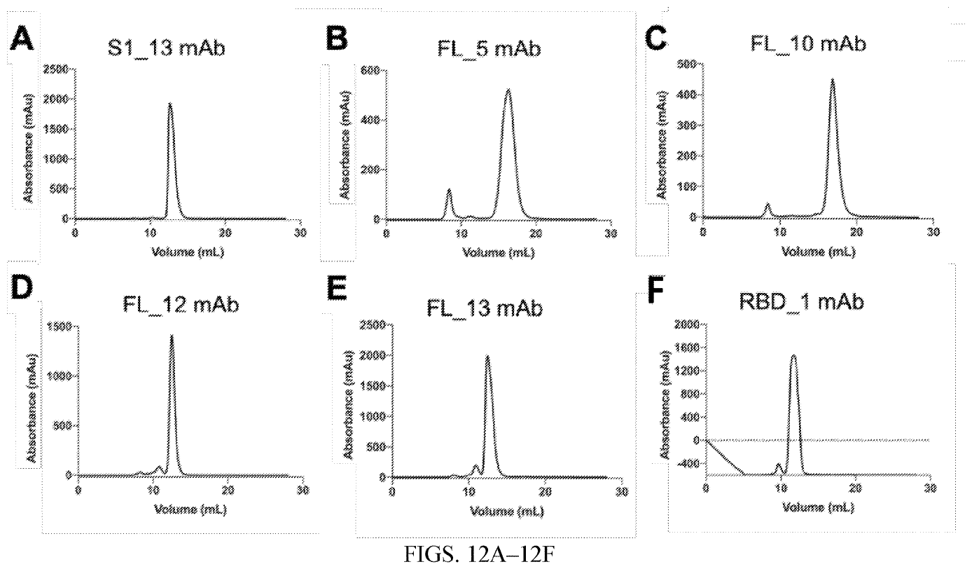

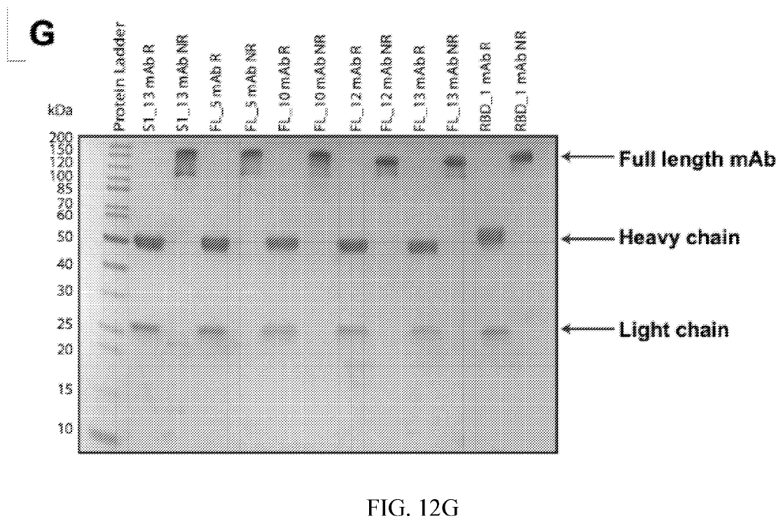

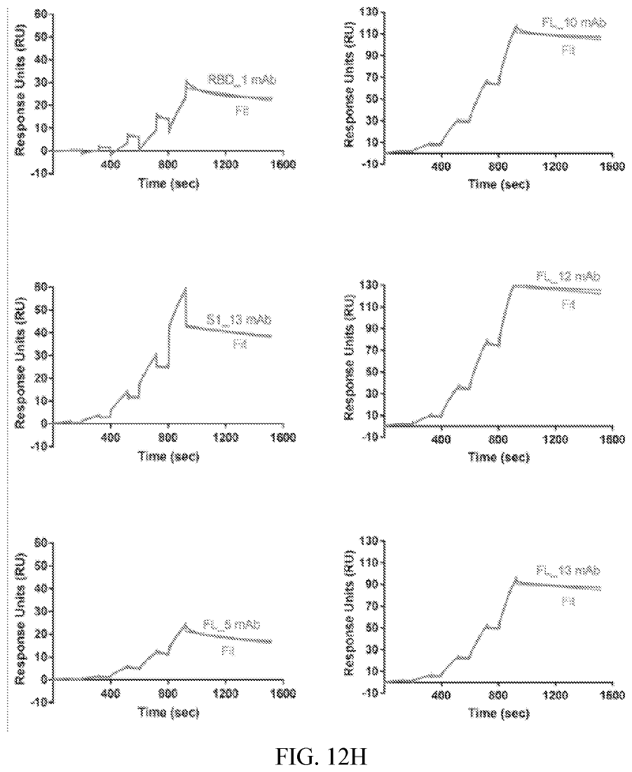

Converting scFvs to full-length antibodies that are more suitable for clinical use can be problematic.²⁷ This issues was addressed by converting the scFv regions from 12 of the phage described above (eight neutralizing and four non-neutralizing) into full-length IgG antibodies rather than antibody fragments (FIG. 3A). All twelve were successfully made into full-length mAbs (examples in FIGS. 12A-12H) and retained specific binding to the SARS-COV-2 spike protein that was equivalent to its phage counterpart (FIG. 3B and FIG. 2B). Moreover, the neutralizing activity was fully retained in six of the eight mAbs, with activities comparable to that of a commercially available, highly neutralizing monoclonal antibody (FIG. 3C and FIG. 3D). It is possible that for the two clones (RBD_3 and S1_4) for which the phage but not the full-length antibodies were neutralizing, steric factors associated with the larger phage interfered with binding of the spike protein to ACE2 but were eliminated in the much smaller antibody format.

### Example 6: Biophysical Characterization and Epitope Mapping of Full-Length Antibodies

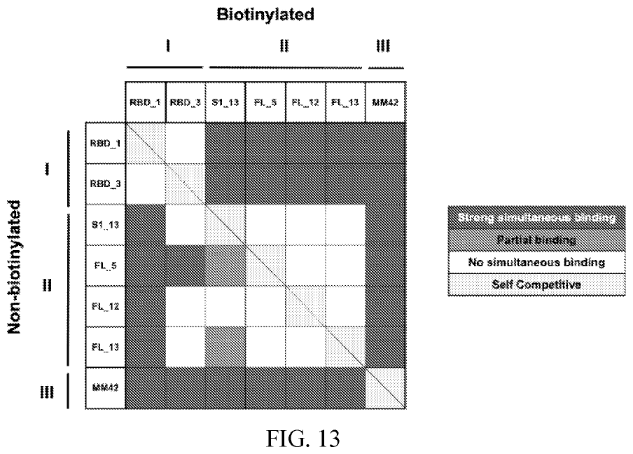

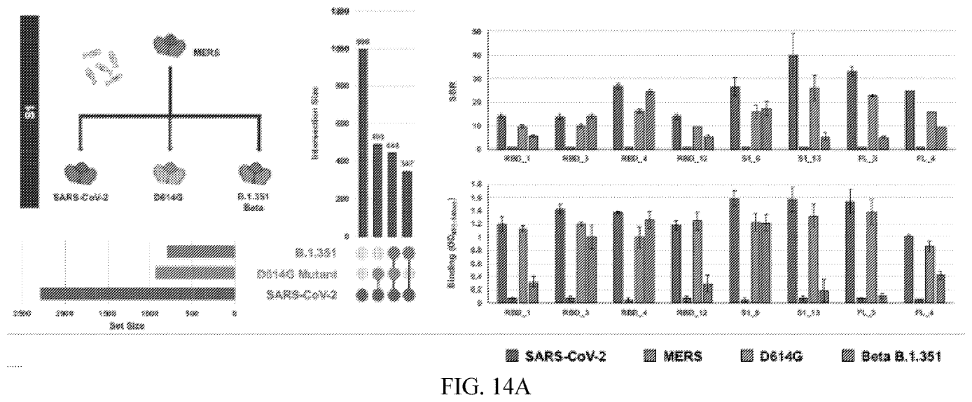

Kinetic and equilibrium constants for the six neutralizing mAbs were measured using Surface Plasmon Resonance (SPR). None of them appeared to bind to the control MERS spike protein, as expected, but all of them bound to the SARS-CoV-2 FL spike protein with high affinity-K_{D}s in the low nanomolar range (6 to 44.2 nM) (FIG. 13 and Table D).

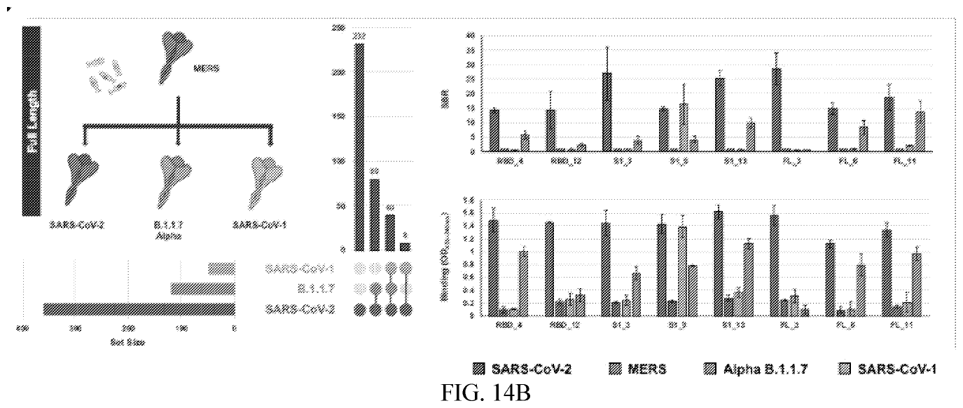

Cocktails composed of more than one neutralizing antibody have been shown to have advantages over a single antibody in prior studies.²⁸⁻³⁰ To evaluate the potential for such combinations with the new mAbs described here, each was labeled with biotin and competed with each of the others for binding to an immobilized RBD protein (FIG. 14A-14B). When two mAbs bind to non-overlapping regions of the RBD, there should be little or no decrease in binding of the first mAb by the second mAb. This enabled the grouping of our six neutralizing mAbs into two groups: Group 1 (RBD_1, RBD_3) and Group 2 (FL_5, FL_12, FL_13, S1_13). Binding of a commercially available non-neutralizing, control RBD antibody was not affected by any of the six neutralizing antibodies, and vice versa.

### Example 7: Assessment of Binding to SARS-COV-2 Variants

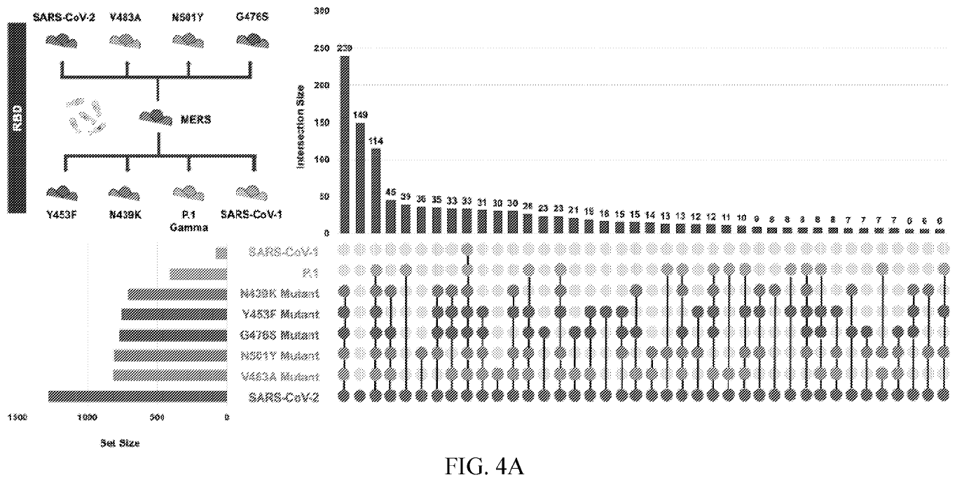

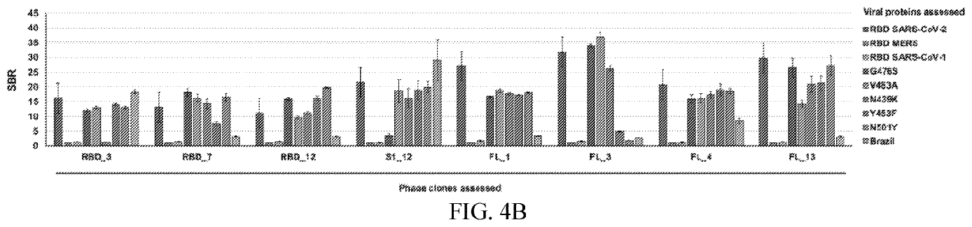

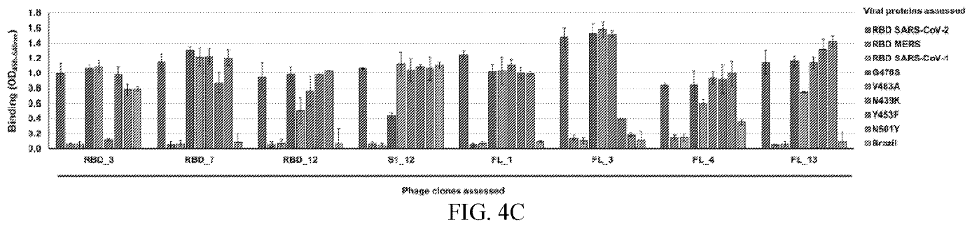

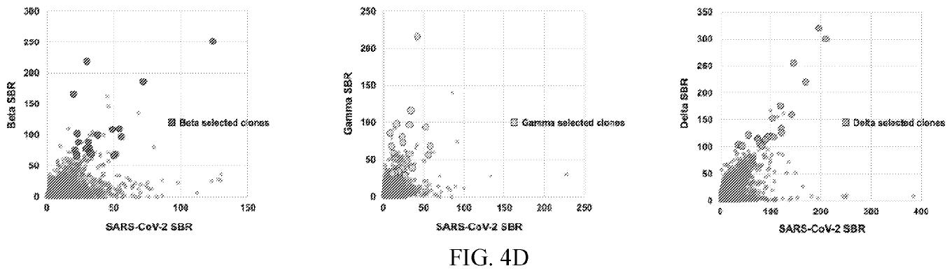

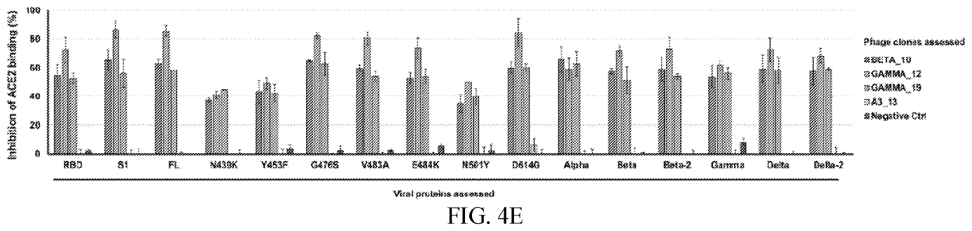

Rather than performing individual ELISAs for millions of clones, it was determined whether SLISY can be used to rapidly identify and predict patterns of binding across multiple variants of SARS-COV-2. To address this question, the phage that survived four rounds of biopanning against the original SARS-COV-2 RBD region were used and SLISY against five variants, each containing a single amino acid change in the RBD, and a pathogenic variant with multiple mutations (Gamma P.1) that emerged during the pandemic was performed. Without any further rounds of biopanning, it was found that 88.3% (1130 of 1279) of clones that bound to the original SARS-COV-2 RBD also bound to at least one of six variants. Many of these phage clones were able to bind to several SARS-COV-2 virus variants; for example, 386 phage clones bound to all five of the single amino acid variants and 147 clones bound to all five single amino variants plus the RBD Gamma variant (FIG. 4A). The differential binding predicted using SLISY was validated by comparing it to ELISA-based testing using phage previously derived from binding to the original SARS-CoV-2 (FIG. 4B and FIG. 4C). This analysis revealed perfect concordance between SLISY and the ELISA. Four other variants of interest were available in either the S1 (D614G and Beta-B.1.351) or FL (SARS-COV-1 and Alpha-B.1.1.7) formats. Similar to above, there was perfect concordance between SLISY and the ELISA based test for both S1 and FL.

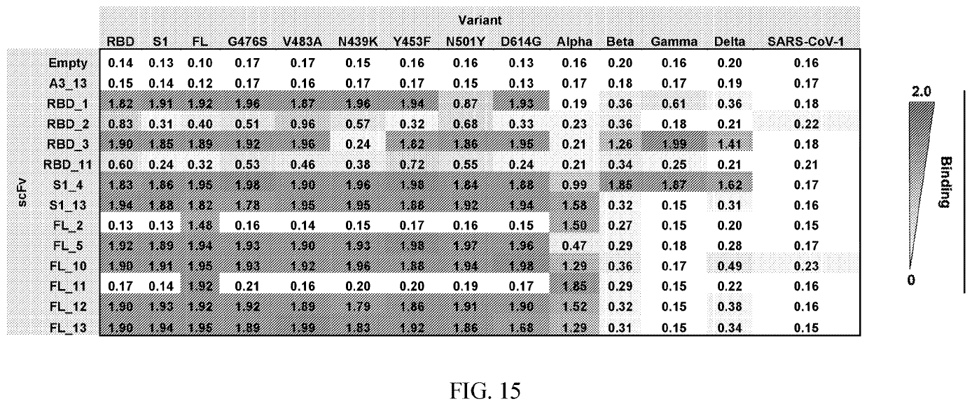

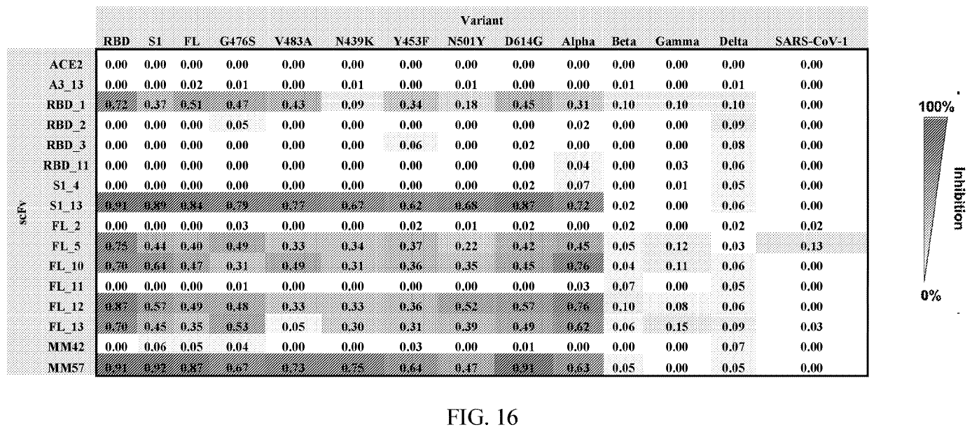

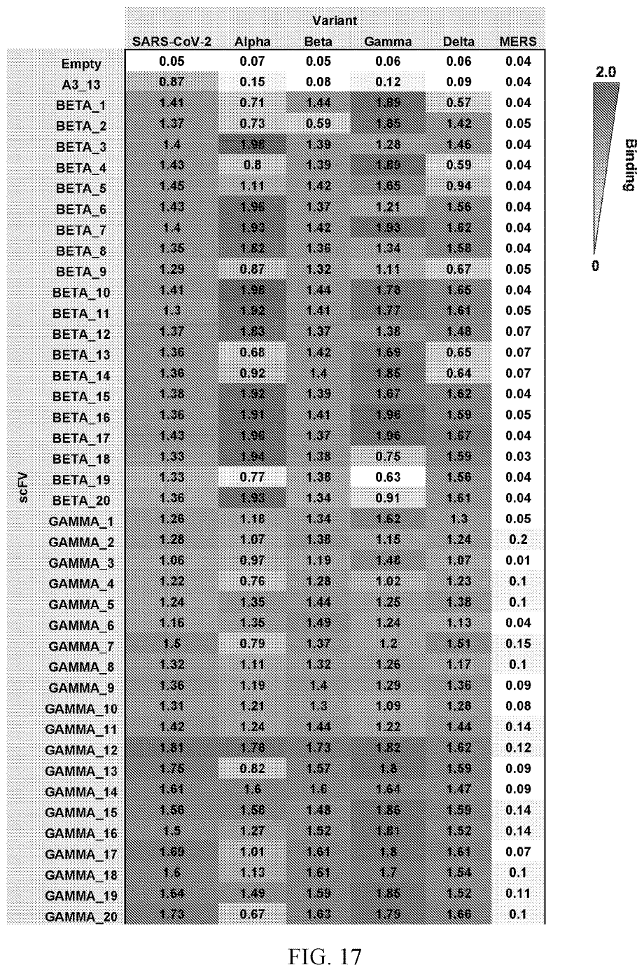

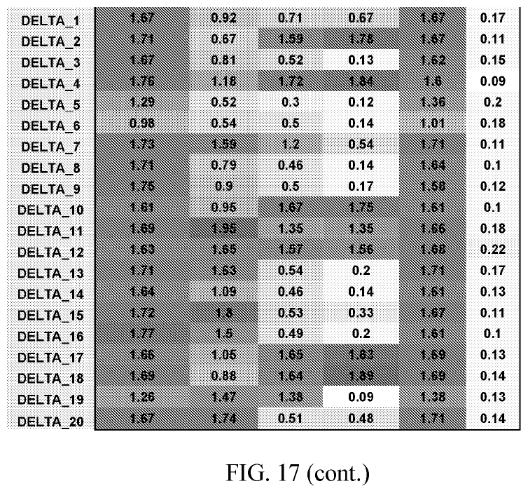

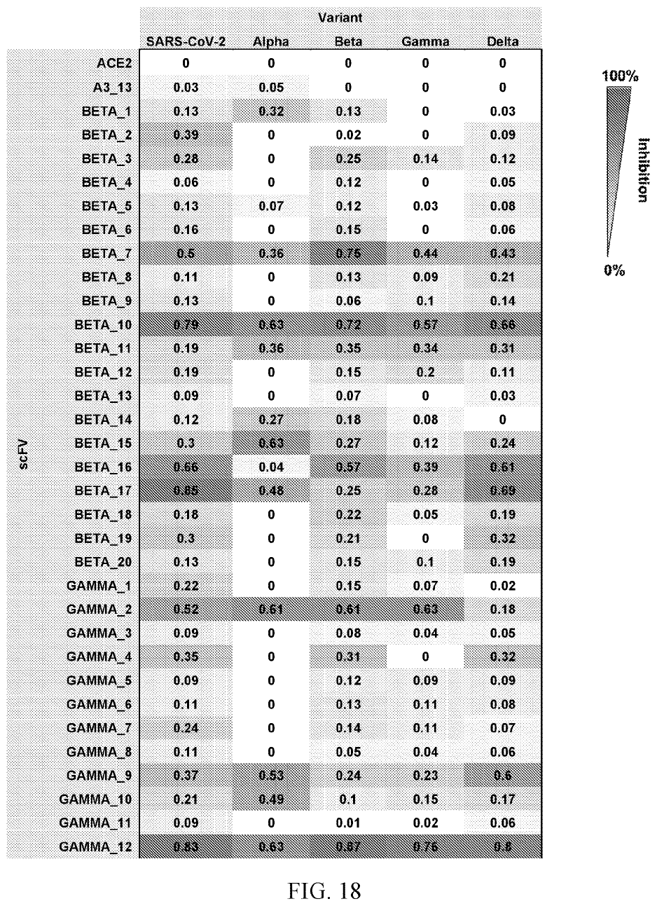

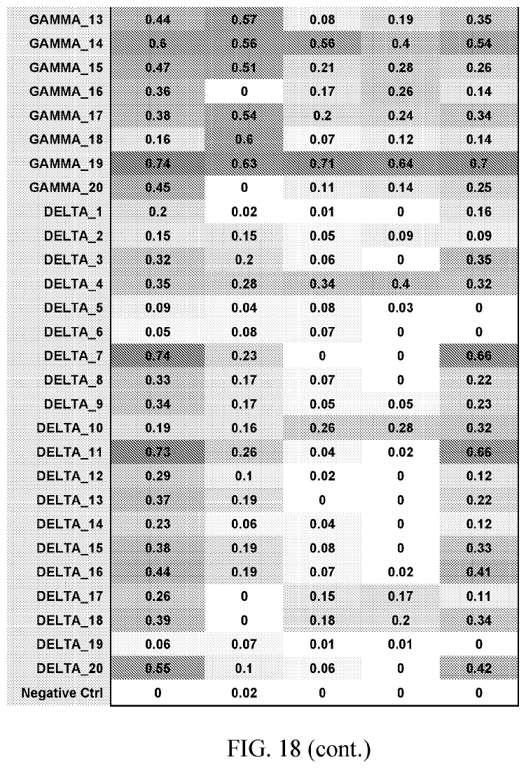

While some of the full-length antibody clones did bind to the variants, none were able to simultaneously neutralize the Beta, Gamma and Delta variants, three viral variants that are highly clinically relevant (FIG. 15 and FIG. 16). Therefore, SLISY from existing biopannings was used to quickly identify new clones against these three variants as well as the original strain (FIG. 4D). Because clones with the most potent neutralizing capacity do not necessarily have the highest SBR, twenty candidate clones against each of the three variants were selected. All tested clones demonstrated strong binding to the original strain and its corresponding variant, with many cross-reactive to multiple variants (FIG. 17). It was also observed that at least 2 out of the 20 clones from each set demonstrated significant inhibition greater than 50% against its variant (FIG. 18). More importantly, clones that were able to neutralize multiple variants were quickly identified. In particular, clones Beta_10, Gamma 12, and Gamma_19 simultaneously inhibited more than 50% of the original, Alpha, Beta, Gamma, and Delta variants (FIG. 4E). By selecting clones using multiple comparisons with SLISY, several broad spectrum “superclones” that warrant further evaluation were rapidly and successfully identified.

In summary, phage clones that could selectively bind to all 14 clinically relevant SARS-COV-2 viral strains tested and three “superclones” that could each bind to every variant were rapidly identify. It is expected that SLISY could be applied to many types of display libraries and used to develop diagnostic and therapeutic proteins, including antibodies, against a variety of important biomedical targets.

### Example 8: Ability of Full-Length Antibodies to Block Variant Pseudovirus Infectivity

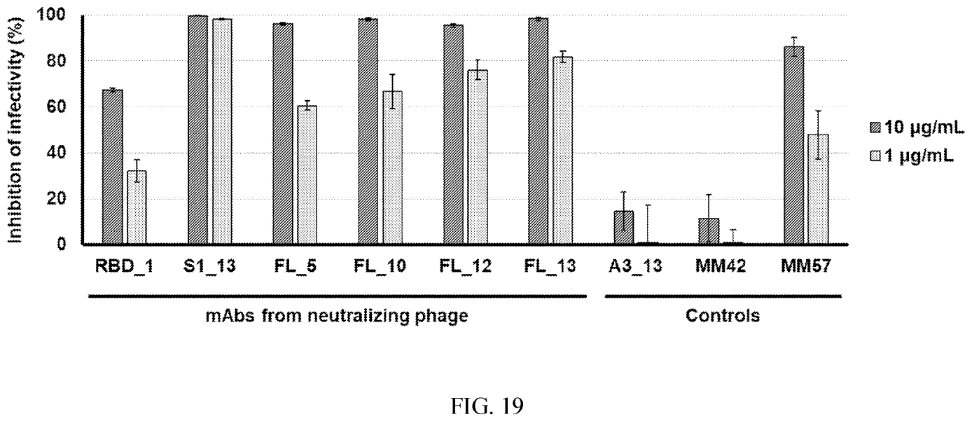

To test if the six neuralizing six mAbs (RBD_1, S1_13, FL_5, FL_10, FL_12, and FL_13) are indeed neutralizing, HEK-293T cells stably expressing ACE2 were incubated simultaneously with lentivirus pseudotyped with the SARS-COV-2 spike glycoprotein and mAbs for 48 hrs. All six mAbs blocked infectivity as measured by luciferase comparable to or better than a commercially available neutralizing mAb indicating that SLISY is able to identify strongly neutralizing mAbs (FIG. 19). Converted full-length antibodies were incubated with pseudovirus expressing SARS-CoV-2 spike protein and ACE2-expressing HEK-293T cells for 48 hours. Infectivity was measured by luciferase activity.

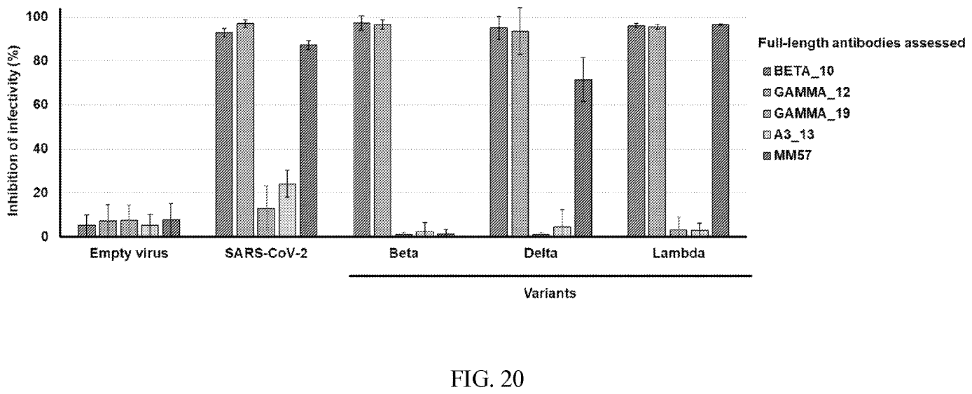

Furthermore, when converted to full-length antibodies, Beta 10 and Gamma 12 were able to block infectivity of original SARS-COV-2 spike pseudovirus as well as variant pseudoviruses of Beta, Delta, and the recently emerged Lambda (FIG. 20). Converted full-length antibodies were incubated with pseudovirus expressing variant SARS-COV-2 spike protein and ACE2-expressing HEK-293T cells for 48 hours. Infectivity was measured by luciferase activity.

### Materials and Methods for Examples 1-7

### Phage Library Construction and Expansion

Methods for scFv phage display library construction and expansion were described previously.^{21,32} Briefly, SS320 bacteria (Lucigen, 60512-2) grown to mid-log phase were supplemented with 2% glucose and infected with M13K07 Helper phage (Antibody Design Labs, PH010L) and library phage. After 1 hr, cells were resuspended in 2×YT medium with carbenicillin (100 μg/mL), kanamycin (50 μg/mL), and IPTG (50 μM) and grown overnight at 30° C. The following morning, the bacterial culture was pelleted at high speed (12,000 g) to obtain clarified supernatant. The phage-laden supernatant was precipitated on ice for 40 min with a 20% PEG-8000/2.5 M NaCl solution at a 1:4 ratio of PEG/NaCl: supernatent. After precipitation, phage was centrifuged at 12,000 g for 40 minutes and resuspended in 1×TBS, 2 mM EDTA. Phage from multiple tubes were pooled, re-precipitated, and resuspended to an average titer of 1×10¹³ cfu/mL. The precipitated phage represents ~250-fold coverage of the library.

### Selection for Phage Binding to HLA-A3 on CFPAC Parental Cells

For round 1 of cell-based panning, 100×10⁶ CFPAC parental cells were harvested and washed in PBS. The cells were directly resuspended in the pooled phage library and incubated at 4° C. for 1 hr with rotation. For the A3 clone spike-in condition, phage clone A3-Clone 20 was initially added to the pooled library for a final amount of 1:100000 phage. After pelleting, the cells were washed 5× in PBS. To recover the phage for reinfection, 1 mL of Elution Buffer (0.2 M glycine at pH 2.2) was used to resuspend cells for 10 min at 20° C. The solution was neutralized with 150 μL of Neutralization Buffer (1 M Tris HCl at pH 9.0). The neutralized phage were then used to infect a 10 mL SS320 culture at mid-log phase with the addition of M13K07 helper phage (MOI of 4) and 2% glucose. As previously described, bacteria were grown overnight and phage particles in conditioned medium were precipitated with PEG/NaCl. Starting in round 2 and for the remainder of biopanning, 15×10⁶ CFPAC HLA A3 knockout cells were washed in PBS and used for negative biopanning. Like positive biopanning, the phage was incubated for 1 hr at 4° C. with rotation before cells were pelleted and the supernatant containing unbound phage was used for positive biopanning. The amount of CFPAC parental cells used for positive biopanning decreased with each round: 15×10⁶ cells for round 2, 10×10⁶ cells for round 3, and 5×10⁶ cells for round 4. At the end of each round, cells were pelleted and phage particles were eluted for infection of overnight SS320 cultures.

### Biopanning for Phage Binding to SARS-COV-2.

His-tagged SARS-COV-2 and MERS antigens (RBD, S1, and FL) were conjugated to HisPur™ Ni-NTA magnetic beads (Thermo Fisher, 88832) at a concentration of 16 μg protein/mg beads following manufacturer's protocol (Table E). In order to remove any phage recognizing a portion of the magnetic beads itself, the precipitated library was in applied to naked 500 μL washed Ni-NTA magnetic beads overnight at 4° C. with rotation. After negative biopanning, the supernatant containing unbound phage was applied to beads conjugated with 10 μg of each SARS-COV-2 protein (RBD, S1, FL) for a total of 30 μg protein for 1 hour at 4° C. The beads were washed 3× with TBST (TBS+0.05% Tween-20) with the last wash overnight at 4° C. Phage were eluted, neutralized, and used to infect bacteria as previously described. Beginning in round 2, we separated the precipitated round 1 phage into three different biopanning strategies corresponding to RBD, S1, or FL. We applied an overnight negative biopanning of 30 μg of the corresponding MERS protein-conjugated beads (i.e. for SARS-COV-2 RBD panning, use MERS RBD) prior to positive biopanning. This remained constant for rounds 3 and 4. However, the amount of SARS-COV-2 protein used for positive biopanning decreased from 4 μg in round 2 to 1 μg in round 3 to 0.5 μg in round 4. After each negative biopanning, the sample was applied to a magnet to isolate the supernatant for positive biopanning. After each round, phage were eluted and used to infect bacteria for overnight cultures.

Sanger Sequencing of Full-Length scFv Phage Clones to Validate Constructs

To identify each clone by Sanger sequencing, 1 μL of bacteria is mixed following manufacturer's protocol with Q5 Hot Start High-Fidelity 2×Master Mix (New England Biolabs, M0494X) and forward and reverse primers (phiS2: 5′-ATGAAATACCTATTGCCTACGG (SEQ ID NO: 395) and psiR2: 5′-CGTTAGTAAATGAATTTTCTGTATGAGG (SEQ ID NO: 396)). The following cycling conditions used are described below:

| | |
|---|---|
| 1 cycle | 1 min at 98 °C |
| 3 cycle3 | 10 s at 98 °C, 10 s at 70 °C, 15 s at 72 °C |
| 3 cycles | 10 s at 98 °C, 10 s at 67 °C, 15 s at 72 °C |
| 3 cycles | 10 s at 98 °C, 10 s at 64 °C, 15 s at 72 °C |
| 3 cycles | 10 s at 98 °C, 10 s at 61 °C, 15 s at 72 °C |
| 20-30 cycles | 10 s at 98 °C, 10 s at 58 °C, 15 s at 72 °C |
| 1 cycle | 5 min at 72 °C |
| | Hold at 10 °C |

PCR products were submitted to Genewiz (South Plainfield, NJ) for Sanger Sequencing. Entire sequence of scFv was identified using SnapGene (San Diego, CA).

### Sequencing-Linked ImmunoSorbent Assay (SLISY)

For cell-based SLISY: For each cell line, CFPAC parental and CFPAC A3 KO, 10×10⁶ cells were trypsinized and washed 3× with PBS. Cell pellets were resuspended in PBS at a concentration of 10×10⁶ cells/mL. Next, 10 μL of polyclonal phage (1013 titer) was applied to both samples and incubated for 1 hr at 20° C. with continuous rotation. Samples were pelleted and washed 3× with PBS. After washing, the samples were resuspended in QuickExtract™ DNA Extraction Solution (Lucigen, QE09050) according to manufacturer's protocol. Samples were passed through the QIAshredder (Qiagen, 79656) and used directly for PCR amplification of CDRH3 region.

For SARS-COV-2 SLISY: His Tag antibody plates (Genscript, L00440C) were individually coated with the spike proteins desired for SLISY comparisons. The spike proteins were diluted in PBS to a concentration of 0.5 μg/mL and incubated at 4° C. for 12 hours. Plates were vigorously washed 6× times with 1×TBST. Next, 100 μL of polyclonal phage (10¹³ titer) was applied to each of the wells for comparison and incubated for 1 hr at 4° C. Next, the plates were washed again 6× times with TBST. After washing, 20 μL of Elution Buffer (0.2 M glycine at pH 2.2) was applied to wells for 20 min at 20° C. Without washing, 3 μL of Neutralization Buffer (1 M Tris HCl at pH 9.0) was directly added to wells and total volume in well (~23 μL) was recovered for PCR amplification.

PCR amplification: Eluted phage was amplified using the following primers (Forward: GGATACCGCTGTCTACTACTGTAGCCG (SEQ ID NO: 1), Reverse: CTGCTCACCGTCACCAATGTGCC (SEQ ID NO: 2) which flank the CDR-H3 region. The sequences at the 5′-ends of these primers incorporated molecular barcodes to facilitate unambiguous enumeration of distinct phage sequences. The protocols for PCR-amplification and sequencing are described in Kinde et al. (2011).²³ Sequences processed and translated using a custom SQL database and both the nucleotide sequences and amino acid translations were analyzed using Microsoft Excel. The SLISY Binding Ratio was calculated by comparing the phage eluted from the target well/cells to the phage eluted from the non-target (negative) well/cells.

$$SLISY Binding Ratio ( SBR ) = \frac{Clone UID in Target Pool}{Clone UID in Negative Pool}$$

Clones that have a UID of zero are given a value of 0.5. Because wells were loaded with equivalent amounts of phage and we were using molecular barcodes to count molecules, we did not normalize for total reads. For comparison, we also calculated a Panning Enrichment Ratio (PER) by dividing the fraction of the phage expressing the specific CDR-H3 in the population after growth of the bound phage by the fraction present in the input material and converting it to log base 2.

### Long-Read Sequencing of Candidate Clones Selected by SLISY

SLISY initially uses sequencing of just the CDR-H3 region of the scFv to determine whether any promising clones exist in the library following biopanning. If so, we used more extensive sequencing to determine the sequence of the entire scFv. In order to obtain the complete variable regions of the heavy and light chains of the scFv, long-read sequencing utilized a custom protocol with three reads (FIG. 5). To efficiently multiplex samples, 96 forward primers were designed with unique well barcodes to serve as sample indexes and a Unique IDentifier (UID) of 14 random nucleotides to serve as molecular barcodes. Thus, each primer contained both a well barcode and the means to identify up to 414 unique molecules per well. Each 25 μl reaction consisted of 12.5 μl of Q5 High-Fidelity 2× Master Mix (New England Biolabs, Ipswich, MA, cat #0491L), 1 μl of 10 μM forward primer (IDT, Coralville, IA), 1 μl of 10 μM reverse primer (IDT), 1 μl of phage, and 9 μl nuclease-free water. Amplification conditions were as follows: 1 denaturation cycle of 98° C. for 3 minutes; amplification cycles of 98° C. for 10 seconds, 61° C. for 2 minutes, and 72° C. for 2 minutes; followed by an infinite hold at 4° C. Amplicons were then sequenced on a MiSeq using the following custom primers designed to sequence all variable regions of the scFv: read 1 primer-5′-ACTGGCCGTCGTTTTACGTCG-3′ (SEQ ID NO: 3), read 2 primer-5′-TGCGCTAATGGTAAAGCGACCTTTCACGCTAT-3′ (SEQ ID NO: 4), and index primer-5′-CTGCAGATGAATAGTCTGCGTGCAGAGGATACAGC-3′ (SEQ ID NO: 5). Run parameters are as follows: read 1-220 cycles, read 2-135 cycles, and index-145 cycles. Reads passing Illumina chastity filters were included in subsequent analysis.

Cloning and Expression of Full-Length Candidate scFvs Identified by SLISY

Based on the long-read sequencing, geneblocks corresponding to the entire scFv were ordered from Integrated DNA Technologies and resuspended at a concentration of 0.05 M in TE Buffer. The pADL-10b phagemid vector was digested using BglI enzyme (New England Biolabs, R0143S) with Antarctic Phosphatase (New England Biolabs, M0289L) in manufacturer recommended Buffer 3.1 for 12 hr at 37° C. Following PCR purification (Qiagen, 28104), 100 ng of linearized pADL-10b plasmid and 1 μL of each resuspended geneblock was added to NEBuilder® HiFi DNA Assembly Master Mix (New England Biolabs, E2621X) for a reaction total volume of 20 μL that is incubated at 50° C. for 1 hr. One μL of the ligation product was mixed with 25 μL of electrocompetent SS320 cells. This mixture was electroporated using a Gene Pulser electroporation system (Bio-Rad, BZA648860) and allowed to recover in Recovery Media (Lucigen, 80026-1) for 1 hr at 37° C. with shaking. Next, 100 μL of 1:500 dilution of transformed cells were plated on 2×YT agar plates supplemented with carbenicillin (100 μg/mL) and 2% glucose. Cells were grown at 37° C. for 12 hours. Individual colonies were inoculated into 200 μl of 2×YT medium containing 100 μg/mL carbenicillin and 2% glucose and grown for three hours at 37° C. The cells were then infected with 1.6×10⁷ M13K07 helper phage and incubated for an additional 1 hr at 37° C. The cells were pelleted, resuspended in 300 μL of 2×YT medium containing carbenicillin (100 μg/mL) and kanamycin (50 μg/mL), and grown overnight at 30° C. Clones were submitted for Sanger sequencing and inserts were confirmed using SnapGene. Once verified, cells were pelleted and 100 μL of phage-laden supernatant was used to infect 9 mL of bacteria to produce phage overnight as described above.

### Flow Cytometry of Cell Lines to Validate HLA-A3-Binding Clones

CFPAC parental and HLA-A3 KO lines were harvested, washed with PBS, and resuspended in ice-cold flow cytometry staining buffer (PBS, 0.5% BSA, 2 mM, EDTA, 0.1% sodium azide) at a concentration of 10×10⁶ cells/mL. Next, 10 μL of precipitated phage (10¹³ titer) was applied to 100 μL of both cells and incubated on ice for 15 min. After washing 3× with staining buffer, cell pellets were resuspended to same concentration (10×10⁶ cells/mL) and stained with 1 μL of rabbit anti-M13 polyclonal antibody (Novus biologicals, Littleton, CO) on ice for 15 min. After washing 3× again, cell pellets were resuspended and stained with 1 μL of PE donkey anti-rabbit IgG antibody (Biolegend, San Diego, CA) for 15 min on ice. After a final wash 3×, stained CFPAC cells were analyzed using a LSRII flow cytometer (Becton Dickinson, Franklin Lakes, NJ) to measure mean fluorescence intensity.

### ELISAs for Binding and Specificity

His Tag antibody plates were coated with a 0.5 μg/mL solution of spike protein antigens diluted in PBS at 4° C. for 12 hours and washed with 1×TBST.

For phage: To test binding and specificity of phage to spike antigens, precipitated phage (10¹³ titer) was diluted 1:1000 in PBS and 100 μL was added to each well and incubated for 1 hr at 4° C. After vigorous washing, the bound phage were then incubated with 100 μL of rabbit anti-M13 antibody diluted 1:5,000 in 1× TBST for 1 hr at 4° C. Following 6 washes with 1×TBST, wells were then incubated with 100 μL of goat anti-rabbit IgG (H+L) antibody HRP (Thermo Fisher, A27036) diluted 1:10,000 in 1×TBST for 1 hr at 4° C.

For converted antibodies: Each converted full-length IgG antibody clones was diluted to 1.0 μg/mL in PBS and 100 μL was added each well with 1 hr incubation at 4° C. Wells were then incubated directly with 100 μL of goat anti-human IgG Fc-HRP antibody (Abcam, ab98624) diluted 1:10,000 in 1×TBST for 1 hr at 4° C. After a 6 more 1×TBST washes, 100 μL of TMB substrate (Biolegend, 421101) was added and allowed to develop. The reaction was quenched with 50 μL of 2 N sulfuric acid. Absorbance at 450 nm and 540 nm was measured with a Synergy H1 Multi-Mode Reader (BioTek, Winooski, VT). O.D. readings were measured as absorbance at 450 nm minus that at 540 nm.

### ELISAs for Measuring Neutralization Potential

His Tag antibody plates were coated with a 0.5 μg/mL solution of spike protein antigens diluted in PBS at 4° C. for 12 hours then washed with 1X TBST. To test blocking, 100 μL of precipitated phage (10¹³ titer) or 10 μg/mL of converted antibody was applied to each well and incubated for 1 hr at 4° C. After vigorous washing, the wells were then incubated with 100 μL of recombinant ACE2-His protein (RayBiotech Life, Inc, Peachtree Corners, GA) for 1 hr at 4° C. After 6×washing, 100 μL of rabbit anti-6×His tag polyclonal antibody (Abcam, ab9108) diluted 1:1,000 in PBS was added and incubated for 1 hr at 4° C. Following 6 washes with 1×TBST, wells were then incubated with 100 μL of goat anti-rabbit IgG (H+L) antibody HRP diluted 1:10,000 in 1×TBST for 1 hr at 4° C. Neutralization ELISAs were developed and measured identical to ELISAs for binding and specificity described above. A negative control antibody, SARS-COV-2 spike mouse Mab (40591-MM42) (Sino Biological, Wayne, PA) that binds to the SARS-COV-2 spike protein but does not neutralize, and a positive control antibody, SARS-COV-2 spike mouse Mab (40592-MM57) (Sino Biological) that does block its interaction with ACE2 were used at 10 μg/mL. An ACE2-His alone condition without phage or antibody served as the baseline signal for spike-ACE2 binding. Neutralization was measured as a decreased in signal from ACE2-His alone.

cPASS: SARS-COV-2 Surrogate Virus Neutralization Test (sVNT)

Assays were performed following manufacturer's protocols for measuring neutralization using the SARS-COV-2 sVNT kit (Genscript). Briefly, 100 μL of 1:1 and 1:10 phage (10¹³ titer) diluted in Sample Dilution Buffer was mixed with diluted HRP-RBD solution with a volume ratio of 1:1. Mixtures were incubated at 37° C. for 30 min. For antibodies, 100 μL of 10 μg/mL and 1.0 μg/mL antibody diluted in Sample Dilution Buffer was mixed with HRP-RBD solution. Provided positive and negative controls were prepared similarly following manufacturer protocols. Next, 100 μL of mixtures were applied to wells pre-coated with recombinant ACE2 protein and incubated at 37° C. for 15 min. After washing plates were 260 μL 1×Wash Solution for four times, 100 μL of provided TMB solution was added and reaction developed for 15 min at 25° C. Reaction was quenched with Stop Solution and plate read at 450 nm and 540 nm absorbance. According to manufacturer's protocol, neutralization was measured as a decrease in signal greater than or equal to 30% of signal from the negative control well.

$$Inhibition = ( 1 - \frac{OD value of Sample}{OD value of Negative Control} ) \times 100 \%$$

### Production and Purification of SARS-COV-2 Specific Antibodies

For IgG Antibodies, the light and heavy chain variable sequences of the scFvs were grafted on the chains of 4D5/trastuzumab and cloned into pcDNA3.4 backbone with a mouse IgKVIII leader signal peptide as we have done before in Hsiue et al. (2021).²¹ Freestyle 293-F cells were transfected with light and heavy chain plasmids with a ratio of 1:1 using PEI at 1:3 at a concentration of 2×10⁶ to 2.5×10⁶ per ml and incubated for 6 days at 37° C. Transfection and expression of the antibodies were carried out at the Eukaryotic Tissue Culture Core Facility at Johns Hopkins University. The media was harvested by centrifugation, filtered through a 0.22 μm PES membrane and purified via protein A affinity chromatography on a HiTrap MabSelect SuRe column (Cytiva, Malborough, MA) with the following running buffer, 20 mM Sodium Phosphate, 150 mM NaCl pH 7.2. The antibodies were eluted with a linear gradient from 0 to 100 mM glycine pH 3.0 over 30 column volumes. Fractions were collected in prefilled tubes with 1 M TRIS pH 9.0. The fractions were quantified by SDS-PAGE gel electrophoresis and the fractions of pure antibody were pooled and dialyzed into 20 mM Sodium Phosphate, 150 mM NaCl pH 7.2. Gel filtration chromatography was used for further purification in 20 mM Sodium Phosphate, 150 mM NaCl pH 7.2 with the Superdex 200 increase, 10/300 GL column (Cytiva). Final fractions were quantified by SDS-PAGE gel electrophoresis and the fractions containing antibody were frozen with liquid nitrogen and stored at −80° C.

### Surface Plasmon Resonance (SPR) Affinity Measurements

SPR experiments were carried out on a Biacore T200 (Cytiva) at 25° C. of a CM5 chip. Protein A/G was diluted (1:25 dilution, 1 μM diluted concentration) in 10 mM sodium acetate buffer at pH 4.5 and immobilized on all flow cells (Fcs) of the CM5 chip to a level of ~4100 response units (RU) using standard amine coupling chemistry. HBS-P (10 mM Hepes pH 7.4, 150 mM NaCl, 0.05% v/v surfactant P20) was used as the immobilization and capture running buffer. Approximately ~70-200 RU of each SLISY-selected antibody was captured onto Fcs 2 through 4. Fcl was used as reference subtraction. Single-cycle kinetics were performed for the analytes binding to the captured ligands in the presence of HBS-P by increasing concentrations (5, 20, 80, 160, and 320 nM, four-fold dilutions) of purified target analytes flowed over Fc 1-4 at a rate at 50 μL/min. The analytes used were SARS-COV-2 RBD, SARS-COV-2 S1, and SARS-COV-2 FL (Table D). The contact and dissociation times were 120 sec and 600 sec, respectively. One 20 sec injection of Glycine pH 2.0 was used for surface regeneration. This regeneration also took away captured ligands. Therefore, ligands were captured in the beginning of every cycle. Binding responses for kinetic analyses were reference and blank subtracted. All curves were fit with a 1:1 kinetic binding model using Biacore Insight evaluation software. All SPR measurements were done in triplicates.

Epitope Binning of SARS-COV-2 mAbs.

An indirect ELISA format was used to identify whether identified neutralizing antibody clones could compete for the same antigenic epitope. Briefly, antibodies were biotinylated and an initial indirect ELISA was performed to calibrate the appropriate concentration (OD value approaches 1.5) of each antibody for the binning assay against the SARS-COV-2 RBD-His. Antibodies with low binding activity on the RBD-His were excluded from downstream assays due to difficulty in showing self-competition. Biotinylated antibody is mixed with each free antibody with the calibrated concentration through a volume ratio of 1:1 and applied to an indirect ELISA format to analyze competition within each pair. Antibody pairs are categorized as likely having the same epitope with one of the competition values (ODmAb1-mAb2) is lower than the self-competition value of the other antibody (ODmAb2) and the other competition value (ODmAb2-mAb1) demonstrates an inhibition of greater than 30% than the biotinylated antibody alone (ODmAb1). Epitope binning assay performed and analyzed by Genscript.

$${Inhibition}_{mAb 1 - mAb 2} = ( 1 - \frac{{OD}_{m A b 1 - m A b 2} - O {D}_{m A b 2}}{O {D}_{m A b 1} - {OD}_{m A b 2}} ) \times 100 \%$$

### Statistics

Unless otherwise indicated, error bars represent the standard deviation of three technical replicates.

### SARS-COV-2 Spike Pseudotyped Lentiviral Assay for Neutralization

SARS-COV-2 S-protein pseudotyped replication incompetent lentiviral particles were produced by first transfecting HEK-293T with GeneJuice transfection reagent (Millipore-Sigma) and SARS-Related Coronavirus 2, Wuhan-Hu-1 Spike-Pseudotyped Lentiviral Kit (Spike-Pseudotyped Lentiviral Kit, NR-52948; BEI Resources Repository, Manassas, VA). Viral supernatant was collected 48 h after transfection and filtered through a 0.45 μM filter. For variant viruses, commercially available pseudotyped luciferase rSARS-COV-2 spike virus for the Beta, Delta, and Lambda strains were obtained from Creative Biosciences (Shirley, NY). 1.25×10⁴ 293T cells engineered to express hACE2 receptor (hACE2.293T cells) were plated on day-1 in black 96-well microplates (Corning, Corning, NY). Parental 293T cells served as a control for nonspecific cell transduction. On Day 0, S-pseudoviral particles+/−the indicated antibodies were added and the plate centrifuged at 800 g for 30′ at 32C. Optimal amounts of S-pseudoviral particles for each virus were titrated up to maximal signal-to-noise without antibodies. Cells were then incubated at 37C in 5% CO2 for 48 h at which time the viral-containing supernatant was aspirated and fresh media containing 150 μg/ml D-Luciferin (Millipore-Sigma) for the original strain or Renilla Luciferase (Promega, Madison, WI) for variant viruses was added. BLI was measured and reactive light units (RLU) determined after subtraction of virus-only background. Inhibition of infectivity by each antibody was calculated as percentage decrease in BLI over baseline (virus without antibodies). The assay was performed in experimental triplicate.

### Data Tables

TABLE A
Selected HLA A3 Clones from Cell-Based Panning

| | | | | SLISY Analysis | | | | | | | | | |
|---|---|---|---|---|---|---|---|---|---|---|---|---|---|
| | | | | | CFP | | CFPA | | | | | | |
| | | | | | AC | | C A3 | | Traditional Analysis | | | | |
| | | | | SBR | Parental | CFPAC | KO | CFPAC | | Inverted | | | |
| | SEQ ID | Starting | Validated | Round | Total | Parental | Total | A3 KO | PER | Frequency | Fold | Baseline | Round 4 |
| CDR-H3 | NO: | Library | Clones | 4 | UIDs | Frequency | UIDs | Frequency | Round 4 | (1 out of n) | Enrichment | Frequency | Frequency |
| YSDGAFD | 6 | Nascent | HLAA3_1 | 43.97 | 22 | 1.92E−05 | 0 | 3.77E−07 | 3.21 | 28254 | 9.28 | 3.81E−06 | 3.54E−05 |
| Y | | | | | | | | | | | | | |
| FYSSYAF | 7 | Nascent | HLAA3_2 | 31.98 | 16 | 1.40E−05 | 0 | 3.77E−07 | 3.70 | 40363 | 12.99 | 1.91E−06 | 2.48E−05 |
| DV | | | | | | | | | | | | | |
| NKYYTA | 8 | Nascent | HLAA3_3 | 29.23 | 117 | 1.02E−04 | 4 | 3.02E−06 | 7.54 | 5651 | 185.65 | 9.53E−07 | 1.77E−04 |
| YYMDY* | | | | | | | | | | | | | |
| GYYASY | 9 | Nascent | HLAA3_4 | 28.43 | 569 | 4.97E−04 | 20 | 1.51E−05 | 8.67 | 2569 | 408.43 | 9.53E−07 | 3.89E−04 |
| YFDV* | | | | | | | | | | | | | |
| RYSSTSA | 10 | Nascent | | 21.99 | 11 | 9.61E−06 | 0 | 3.77E−07 | 1.89 | 282543 | 3.71 | 9.53E−07 | 3.54E−06 |
| FDY | | | | | | | | | | | | | |
| GTYFAM | 11 | Nascent | HLAA3_5 | 20.99 | 273 | 2.39E−04 | 13 | 9.81E−06 | 7.75 | 4871 | 215.36 | 9.53E−07 | 2.05E−04 |
| DY* | | | | | | | | | | | | | |
| GFSYYKG | 12 | Nascent | | 19.99 | 20 | 1.75E−05 | 1 | 7.54E−07 | 3.48 | 94181 | 11.14 | 9.53E−07 | 1.06E−05 |
| YFDY | | | | | | | | | | | | | |
| SLSSYLP | 13 | Nascent | | 19.99 | 10 | 8.74E−06 | 0 | 3.77E−07 | 3.48 | 94181 | 11.14 | 9.53E−07 | 1.06E−05 |
| YTSYFAY | | | | | | | | | | | | | |
| ASSAYAF | 14 | Nascent | | 17.99 | 18 | 1.57E−05 | 1 | 7.54E−07 | 0.89 | 94181 | 1.86 | 5.72E−06 | 1.06E−05 |
| DY | | | | | | | | | | | | | |
| SWFSKSA | 15 | Nascent | HLAA3_9 | 13.99 | 28 | 2.45E−05 | 2 | 1.51E−06 | 3.48 | 94181 | 11.14 | 9.53E−07 | 1.06E−05 |
| MDY* | | | | | | | | | | | | | |
| WGSYAF | 16 | Nascent | | 13.99 | 7 | 6.12E−06 | 0 | 3.77E−07 | 2.31 | 35318 | 4.95 | 5.72E−06 | 2.83E−05 |
| DY | | | | | | | | | | | | | |
| ALYSAYF | 17 | Nascent | HLAA3_6 | 13.49 | 27 | 2.36E−05 | 2 | 1.51E−06 | 5.80 | 18836 | 55.70 | 9.53E−07 | 5.31E−05 |
| AY | | | | | | | | | | | | | |
| GDYYYS | 18 | Nascent | HLAA3_7 | 13.49 | 54 | 4.72E−05 | 4 | 3.02E−06 | 6.70 | 10091 | 103.97 | 9.53E−07 | 9.91E−05 |
| YLDY | | | | | | | | | | | | | |
| GLSSHSA | 19 | Nascent | | 11.99 | 12 | 1.05E−05 | 1 | 7.54E−07 | 1.89 | 282543 | 3.71 | 9.53E−07 | 3.54E−06 |
| FDY | | | | | | | | | | | | | |
| GFYYYY | 20 | Nascent | | 11.99 | 6 | 5.24E−06 | 0 | 3.77E−07 | 2.89 | 141271 | 7.43 | 9.53E−07 | 7.08E−06 |
| YSYYMA | | | | | | | | | | | | | |
| MDV | | | | | | | | | | | | | |
| YMAWSY | 21 | Nascent | | 11.99 | 6 | 5.24E−06 | 0 | 3.77E−07 | 1.89 | 282543 | 3.71 | 9.53E−07 | 3.54E−06 |
| KYMDY | | | | | | | | | | | | | |
| ATSYSHS | 22 | Nascent | | 11.24 | 45 | 3.93E−05 | 4 | 3.02E−06 | 6.28 | 13454 | 77.97 | 9.53E−07 | 7.43E−05 |
| YSIYYHY | | | | | | | | | | | | | |
| YMDY | | | | | | | | | | | | | |
| SFSRYGY | 23 | Nascent | HLAA3_8 | 10.21 | 3830 | 3.35E−03 | 375 | 2.83E−04 | 11.12 | 472 | 2224.11 | 9.53E−07 | 2.12E−03 |
| MDY | | | | | | | | | | | | | |
| QMYYEY | 24 | Nascent | | 9.99 | 5 | 4.37E−06 | 0 | 3.77E−07 | 3.48 | 94181 | 11.14 | 9.53E−07 | 1.06E−05 |
| MDY | | | | | | | | | | | | | |
| SYGSYM | 25 | Nascent | | 9.99 | 5 | 4.37E−06 | 0 | 3.77E−07 | -0.43 | 141271 | 0.74 | 9.53E−06 | 7.08E−06 |
| DY | | | | | | | | | | | | | |
| YYGYAM | 26 | Nascent | | 9.99 | 5 | 4.37E−06 | 0 | 3.77E−07 | -0.31 | 14127 | 0.81 | 8.77E−05 | 7.08E−05 |
| DY | | | | | | | | | | | | | |
| SFSSYGY | 27 | Nascent | | 8.77 | 79 | 6.90E−05 | 9 | 6.79E−06 | 5.89 | 17659 | 59.41 | 9.53E−07 | 5.66E−05 |
| MDY | | | | | | | | | | | | | |
| ETMYGF | 28 | Nascent | | 7.99 | 16 | 1.40E−05 | 2 | 1.51E−06 | 4.70 | 40363 | 25.99 | 9.53E−07 | 2.48E−05 |
| DY* | | | | | | | | | | | | | |
| GTSSHSA | 29 | Nascent | | 7.99 | 16 | 1.40E−05 | 2 | 1.51E−06 | 1.89 | 282543 | 3.71 | 9.53E−07 | 3.54E−06 |
| FDY | | | | | | | | | | | | | |
| PLYSSFY | 30 | Nascent | | 7.99 | 8 | 6.99E−06 | 1 | 7.54E−07 | 4.21 | 56509 | 18.57 | 9.53E−07 | 1.77E−05 |
| SAMDY | | | | | | | | | | | | | |
| DSYYYM | 31 | Nascent | | 7.99 | 4 | 3.49E−06 | 0 | 3.77E−07 | 0.21 | 56509 | 1.16 | 1.53E−05 | 1.77E−05 |
| DY | | | | | | | | | | | | | |
| GFTWRY | 32 | Nascent | | 7.99 | 4 | 3.49E−06 | 0 | 3.77E−07 | 4.70 | 40363 | 25.99 | 9.53E−07 | 2.48E−05 |
| FDV | | | | | | | | | | | | | |
| GGIPYAF | 33 | Nascent | | 7.99 | 4 | 3.49E−06 | 0 | 3.77E−07 | 2.89 | 141271 | 7.43 | 9.53E−07 | 7.08E−06 |
| DV | | | | | | | | | | | | | |
| GPSSHSA | 34 | Nascent | | 7.99 | 4 | 3.49E−06 | 0 | 3.77E−07 | 1.89 | 282543 | 3.71 | 9.53E−07 | 3.54E−06 |
| FVY | | | | | | | | | | | | | |
| SGSYYFD | 35 | Nascent | | 7.99 | 4 | 3.49E−06 | 0 | 3.77E−07 | -0.31 | 28254 | 0.81 | 4.39E−05 | 3.54E−05 |
| Y | | | | | | | | | | | | | |
| SWGVAM | 36 | Nascent | | 7.99 | 4 | 3.49E−06 | 0 | 3.77E−07 | 5.06 | 31394 | 33.42 | 9.53E−07 | 3.19E−05 |
| DY | | | | | | | | | | | | | |
| SYTSYM | 37 | Nascent | | 7.99 | 4 | 3.49E−06 | 0 | 3.77E−07 | 0.57 | 70636 | 1.48 | 9.53E−06 | 1.42E−05 |
| DY | | | | | | | | | | | | | |
| VGYYYF | 38 | Nascent | | 7.99 | 4 | 3.49E−06 | 0 | 3.77E−07 | 0.31 | 141271 | 1.24 | 5.72E−06 | 7.08E−06 |
| DY | | | | | | | | | | | | | |
| WYSRGY | 39 | A3- | | 79.99 | 40 | 1.43E−05 | 0 | 1.61E−07 | 5.24 | 13241 | 37.79 | 2.00E−06 | 7.55E−05 |
| MDY | | Clone20 | | | | | | | | | | | |
| | | spike-in | | | | | | | | | | | |
| QYGYPY | 40 | A3- | HLAA3_11 | 77.49 | 155 | 5.53E−05 | 2 | 6.45E−07 | 5.35 | 12260 | 40.81 | 2.00E−06 | 8.16E−05 |
| YFDY | | Clone20 | | | | | | | | | | | |
| | | spike-in | | | | | | | | | | | |
| GTYFAM | 41 | A3- | HLAA3_5 | 36.54 | 402 | 1.43E−04 | 11 | 3.55E−06 | 7.87 | 2136 | 234.27 | 2.00E−06 | 4.68E−04 |
| DY* | | Clone20 | | | | | | | | | | | |
| | | spike-in | | | | | | | | | | | |
| ESMYAM | 42 | A3- | | 32.00 | 32 | 1.14E−05 | 1 | 3.22E−07 | 4.45 | 11415 | 21.91 | 4.00E−06 | 8.76E−05 |
| DY | | Clone20 | | | | | | | | | | | |
| | | spike-in | | | | | | | | | | | |
| ETMYGF | 43 | A3- | | 30.00 | 30 | 1.07E−05 | 1 | 3.22E−07 | 4.18 | 27586 | 18.14 | 2.00E−06 | 3.63E−05 |
| DY* | | Clone20 | | | | | | | | | | | |
| | | spike-in | | | | | | | | | | | |
| SNYSSYY | 44 | A3- | | 24.57 | 172 | 6.13E−05 | 7 | 2.26E−06 | 6.82 | 4414 | 113.36 | 2.00E−06 | 2.27E−04 |
| LDY | | Clone20 | | | | | | | | | | | |
| | | spike-in | | | | | | | | | | | |
| NKYYTA | 45 | A3- | HLAA3_3 | 23.67 | 213 | 7.59E−05 | 9 | 2.90E−06 | 7.20 | 3413 | 146.61 | 2.00E−06 | 2.93E−04 |
| YYMDY* | | Clone20 | | | | | | | | | | | |
| | | spike-in | | | | | | | | | | | |
| GYYASY | 46 | A3- | HLAA3_4 | 23.04 | 599 | 2.14E−04 | 26 | 8.38E−06 | 7.39 | 2982 | 167.77 | 2.00E−06 | 3.35E−04 |
| YFDV* | | Clone20 | | | | | | | | | | | |
| | | spike-in | | | | | | | | | | | |
| DVKSYR | 47 | A3- | | 23.00 | 69 | 2.46E−05 | 3 | 9.67E−07 | 3.18 | 55172 | 9.07 | 2.00E−06 | 1.81E−05 |
| NYFDY | | Clone20 | | | | | | | | | | | |
| | | spike-in | | | | | | | | | | | |
| SPWYYL | 48 | A3- | | 22.00 | 44 | 1.57E−05 | 2 | 6.45E−07 | 5.18 | 13793 | 36.27 | 2.00E−06 | 7.25E−05 |
| DY | | Clone20 | | | | | | | | | | | |
| | | spike-in | | | | | | | | | | | |
| SLWYYM | 49 | A3- | | 18.00 | 9 | 3.21E−06 | 0 | 1.61E−07 | 1.60 | 165515 | 3.02 | 2.00E−06 | 6.04E−06 |
| DY | | Clone20 | | | | | | | | | | | |
| | | spike-in | | | | | | | | | | | |
| SPWDYM | 50 | A3- | | 18.00 | 9 | 3.21E−06 | 0 | 1.61E−07 | 0.60 | 331030 | 1.51 | 2.00E−06 | 3.02E−06 |
| DY | | Clone20 | | | | | | | | | | | |
| | | spike-in | | | | | | | | | | | |
| SWFSKSA | 51 | A3- | HLAA3_9 | 16.67 | 100 | 3.56E−05 | 6 | 1.93E−06 | 5.95 | 8074 | 61.97 | 2.00E−06 | 1.24E−04 |
| MDY* | | Clone20 | | | | | | | | | | | |
| | | spike-in | | | | | | | | | | | |
| DSVGYAF | 52 | A3- | | 16.00 | 8 | 2.85E−06 | 0 | 1.61E−07 | 3.18 | 55172 | 9.07 | 2.00E−06 | 1.81E−05 |
| DY | | Clone20 | | | | | | | | | | | |
| | | spike-in | | | | | | | | | | | |
| DMYSVR | 53 | A3- | HLAA3_10 | 14.40 | 72 | 2.57E−05 | 5 | 1.61E−06 | 5.30 | 12732 | 39.30 | 2.00E−06 | 7.85E−05 |
| SAMDY | | Clone20 | | | | | | | | | | | |
| | | spike-in | | | | | | | | | | | |
| SMYQYSS | 54 | A3- | | 13.00 | 13 | 4.63E−06 | 1 | 3.22E−07 | 3.40 | 47290 | 10.58 | 2.00E−06 | 2.11E−05 |
| FDY | | Clone20 | | | | | | | | | | | |
| | | spike-in | | | | | | | | | | | |
| SRYTRYY | 55 | A3- | | 12.75 | 102 | 3.64E−05 | 8 | 2.58E−06 | 5.84 | 8711 | 57.43 | 2.00E−06 | 1.15E−04 |
| MDY | | Clone20 | | | | | | | | | | | |
| | | spike-in | | | | | | | | | | | |
| ESYYGLD | 56 | A3- | | 12.00 | 6 | 2.14E−06 | 0 | 1.61E−07 | 4.18 | 27586 | 18.14 | 2.00E−06 | 3.63E−05 |
| Y | | Clone20 | | | | | | | | | | | |
| | | spike-in | | | | | | | | | | | |
| ATSYTYF | 57 | A3- | | 9.50 | 76 | 2.71E−05 | 8 | 2.58E−06 | 6.18 | 6896 | 72.55 | 2.00E−06 | 1.45E−04 |
| DV | | Clone20 | | | | | | | | | | | |
| | | spike-in | | | | | | | | | | | |
| FYSGYG | 58 | A3- | | 8.20 | 82 | 2.92E−05 | 10 | 3.22E−06 | 5.95 | 8074 | 61.97 | 2.00E−06 | 1.24E−04 |
| MDY | | Clone20 | | | | | | | | | | | |
| | | spike-in | | | | | | | | | | | |
| AFSYYYY | 59 | A3- | | 8.00 | 4 | 1.43E−06 | 0 | 1.61E−07 | 0.60 | 331030 | 1.51 | 2.00E−06 | 3.02E−06 |
| YSMYYY | | Clone20 | | | | | | | | | | | |
| AVDY | | spike-in | | | | | | | | | | | |
| AYWYAF | 60 | A3- | | 8.00 | 1 | 1.43E−06 | 0 | 1.61E−07 | 0.60 | 165515 | 1.51 | 4.00E−06 | 6.04E−06 |
| DY | | Clone20 | | | | | | | | | | | |
| | | spike-in | | | | | | | | | | | |
| DNYYFA | 61 | A3- | | 8.00 | 4 | 1.43E−06 | 0 | 1.61E−07 | 0.60 | 331030 | 1.51 | 2.00E−06 | 3.02E−06 |
| MDY | | Clone20 | | | | | | | | | | | |
| | | spike-in | | | | | | | | | | | |
| GFSAIDG | 62 | A3- | | 8.00 | 4 | 1.43E−06 | 0 | 1.61E−07 | 1.60 | 165515 | 3.02 | 2.00E−06 | 6.04E−06 |
| YYYYFD | | Clone20 | | | | | | | | | | | |
| V | | spike-in | | | | | | | | | | | |
| GSWYYS | 63 | A3- | | 8.00 | 4 | 1.43E−06 | 0 | 1.61E−07 | 0.60 | 331030 | 1.51 | 2.00E−06 | 3.02E−06 |
| YSYYYYS | | Clone20 | | | | | | | | | | | |
| GYDYYM | | spike-in | | | | | | | | | | | |
| DV | | | | | | | | | | | | | |
| GTYFALD | 64 | A3- | | 8.00 | 4 | 1.43E−06 | 0 | 1.61E−07 | 0.60 | 331030 | 1.51 | 2.00E−06 | 3.02E−06 |
| Y | | Clone20 | | | | | | | | | | | |
| | | spike-in | | | | | | | | | | | |
| GYSRYSF | 65 | A3- | | 8.00 | 4 | 1.43E−06 | 0 | 1.61E−07 | 0.60 | 331030 | 1.51 | 2.00E−06 | 3.02E−06 |
| DY | | Clone20 | | | | | | | | | | | |
| | | spike-in | | | | | | | | | | | |
| PHSYQPA | 66 | A3- | | 8.00 | 4 | 1.43E−06 | 0 | 1.61E−07 | 0.60 | 331030 | 1.51 | 2.00E−06 | 3.02E−06 |
| FDY | | Clone20 | | | | | | | | | | | |
| | | spike-in | | | | | | | | | | | |
| SAYYAV | 67 | A3- | | 8.00 | 4 | 1.43E−06 | 0 | 1.61E−07 | 2.05 | 30094 | 4.16 | 8.00E−06 | 3.32E−05 |
| DY | | Clone20 | | | | | | | | | | | |
| | | spike-in | | | | | | | | | | | |
| SWYYAF | 68 | A3- | | 8.00 | 4 | 1.43E−06 | 0 | 1.61E−07 | -0.20 | 22069 | 0.87 | 5.20E−05 | 4.53E−05 |
| DY | | Clone20 | | | | | | | | | | | |
| | | spike-in | | | | | | | | | | | |
| YYAYSFD | 69 | A3- | | 8.00 | 4 | 1.43E−06 | 0 | 1.61E−07 | -1.99 | 331030 | 0.25 | 1.20E−05 | 3.02E−06 |
| Y | | Clone20 | | | | | | | | | | | |
| | | spike-in | | | | | | | | | | | |
| YYSSAFD | 70 | A3- | | 8.00 | 4 | 1.43E−06 | 0 | 1.61E−07 | -1.47 | 15763 | 0.36 | 1.76E−04 | 6.34E−05 |
| Y | | Clone20 | | | | | | | | | | | |
| | | spike-in | | | | | | | | | | | |
| SPWYYM | 71 | A3- | | 7.67 | 23 | 8.20E−06 | 3 | 9.67E−07 | 5.81 | 8947 | 55.92 | 2.00E−06 | 1.12E−04 |
| EY | | Clone20 | | | | | | | | | | | |
| | | spike-in | | | | | | | | | | | |
| SPCYYM | 72 | A3- | | 7.27 | 109 | 3.89E−05 | 15 | 4.84E−06 | 5.12 | 14393 | 34.76 | 2.00E−06 | 6.95E−05 |
| DY | | Clone20 | | | | | | | | | | | |
| | | spike-in | | | | | | | | | | | |
| SPWYYM | 73 | A3- | A3- | 7.02 | 12832 | 4.57E−03 | 1829 | 5.90E−04 | 11.49 | 15 | 2868.36 | 2.36E−05 | 6.78E−02 |
| DY | | Clone20 | Clone20 | | | | | | | | | | |
| | | spike-in | | | | | | | | | | | |

*Indicates clones that appeared in both bio-panning strategies.
** Eleven representative clones with SBRs higher than 10 were randomly selected for validation. The average SBR of selected clones was 26.51 (range 10.21 to 77.49) whereas the average SBR of 36 clones with an SBRs greater than 10 was 23.33 (range 10.21 to 79.99).

TABLE B
Amino Acid Sequence of HLA A3 scFvs tested

| | | SEQ | | SEQ |
|---|---|---|---|---|
| | CDR- | ID | Amino acid sequence of | ID |
| Clone | H3 | NO: | scFv* | NO: |
| A3- | SPWYY | 74 | QQKPGKAPKLLIYSASFLYSGVPSR | 75 |
| Clone20 | MDY | | FSGSRSGTDFTLTISSLQPEDFATY | |
| | | | YCQQSWTYPVTFGQGTKVEIKRT | |
| | | | GGGSGGGGSGGGASEVQLVESGG | |
| | | | GLVQPGGSLRLSCAASGFNIYPYR | |
| | | | MHWVRQAPGKGLEWVADILPGN | |
| | | | GSTSYADSVKGRFTISADTSKNTA | |
| | | | YLQMNSLRAEDTAVYYCSRSPWY | |
| | | | YMDYWGQGTLVTVSSGGGASDY | |
| | | | KDDDDK | |
| HLAA3_1 | YSDGA | 76 | QQKPGKAPKLLIYSASFLYSGVPSR | 77 |
| | FDY | | FSGSRSGTDFTLTISSLQPEDFATY | |
| | | | YCQQTYRTPITFGQGTKVEIKRTG | |
| | | | GGSGGGGSGGGASEVQLVESGGG | |
| | | | LVQPGGSLRLSCAASGFNVQGDG | |
| | | | MHWVRQAPGKGLEWVAMVGPYS | |
| | | | SYTYYADSVKGRFTISADTSKNTA | |
| | | | YLQMNSLRAEDTAVYYCSRYSDG | |
| | | | AFDYWGQGTLVTVSSGGGASDYK | |
| | | | DDDDK | |
| HLAA3_2 | FYSSY | 78 | QQKPGKAPKLLIYSASFLYSGVPSR | 79 |
| | AFDV | | FSGSRSGTDFTLTISSLQPEDFATY | |
| | | | YCQQSYRSPITFGQGTKVEIKRTG | |
| | | | GGSGGGGSGGGASEVQLVESGGG | |
| | | | LVQPGGSLRLSCAASGFNITKYSIH | |
| | | | WVRQAPGKGLEWVAMVGPYSSY | |
| | | | TYYADSVKGRFTISADTSKNTAYL | |
| | | | QMNSLRAEDTAVYYCSRFYSSYA | |
| | | | FDVWGQGTLVTVSSGGGASDYKD | |
| | | | DDDK | |
| HLAA3_3 | NKYYT | 80 | QQKPGKAPKLLIYSASFLYSGVPSR | 81 |
| | AYYMD | | FSGSRSGTDFTLTISSLQPEDFATY | |
| | Y | | YCQQYYVRPITFGQGTKVEIKRTG | |
| | | | GGSGGGGSGGGASEVQLVESGGG | |
| | | | LVQPGGSLRLSCAASGFNIYSYAM | |
| | | | HWVRQAPGKGLEWVASVAPGSG | |
| | | | YTYYADSVKGRFTISADTSKNTAY | |
| | | | LQMNSLRAEDTAVYYCSRNKYYT | |
| | | | AYYMDYWGQGTLVTVSSGGGAS | |
| | | | DYKDDDDK | |
| HLAA3_4 | GYYAS | 82 | QQKPGKAPKLLIYSASFLYSGVPSR | 83 |
| | YYFDV | | FSGSRSGTDFTLTISSLQPEDFATY | |
| | | | YCQQYHFKPMTFGQGTKVEIKRT | |
| | | | GGGSGGGGSGGGASEVQLVESGG | |
| | | | GLVQPGGSLRLSCAASGFNIYSYA | |
| | | | MHWVRQAPGKGLEWVASFVPGS | |
| | | | YATYYADSVKGRFTISADTSKNTA | |
| | | | YLQMNSLRAEDTAVYYCSRGYYA | |
| | | | SYYFDVWGQGTLVTVSSGGGASD | |
| | | | YKDDDDK | |
| HLAA3_5 | GTYFA | 84 | QQKPGKAPKLLIYSASFLYSGVPSR | 85 |
| | MDY | | FSGSRSGTDFTLTISSLQPEDFATY | |
| | | | YCQQFSSYPITFGQGTKVEIKRTGG | |
| | | | GSGGGGSGGGASEVQLVESGGGL | |
| | | | VQPGGSLRLSCAASGFNLSYYWM | |
| | | | HWVRQAPGKGLEWVASIYPDGSY | |
| | | | TWYADSVKGRFTISADTSKNTAYL | |
| | | | QMNSLRAEDTAVYYCSRGTYFA | |
| | | | MDYWGQGTLVTVSSGGGASDYK | |
| | | | DDDDK | |
| HLAA3_6 | ALYSA | 86 | QQKPGKAPKLLIYSASFLYSGVPSR | 87 |
| | YFAY | | FSGSRSGTDFTLTISSLQPEDFATY | |
| | | | YCQQYYRAPPTFGQGTKVEIKRTG | |
| | | | GGSGGGGSGGGASEVQLVESGGG | |
| | | | LVQPGGSLRLSCAASGFNIFGYYM | |
| | | | HWVRQAPGKGLEWVAVVSPGSG | |
| | | | YTNYADSVKGRFTISADTSKNTAY | |
| | | | LQMNSLRAEDTAVYYCSRALYSA | |
| | | | YFAYWGQGTLVTVSSGGGASDYK | |
| | | | DDDDK | |
| HLAA3_7 | GDYYY | 88 | QQKPGKAPKLLIYSASFLYSGVPSR | 89 |
| | SYLDY | | FSGSRSGTDFTLTISSLQPEDFATY | |
| | | | YCQQWETYPATFGQGTKVEIKRT | |
| | | | GGGSGGGGSGGGASEVQLVESGG | |
| | | | GLVQPGGSLRLSCAASGFNVYYY | |
| | | | WMHWVRQAPGKGLEWVASIYPD | |
| | | | SDNTYYADSVKGRFTISADTSKNT | |
| | | | AYLQMNSLRAEDTAVYYCSRGDY | |
| | | | YYSYLDYWGQGTLVTVSSGGGAS | |
| | | | DYKDDDDK | |
| HLAA3_8 | SFSRY | 90 | QQKPGKAPKLLIYSASFLYSGVPSR | 91 |
| | GYMDY | | FSGSRSGTDFTLTISSLQPEDFATY | |
| | | | YCQQSWYPPITFGQGTKVEIKRTG | |
| | | | GGSGGGGSGGGASEVQLVESGGG | |
| | | | LVQPGGSLRLSCAASGFNIYSYYIH | |
| | | | WVRQAPGKGLEWVAKIYPDSGYT | |
| | | | YYADSVKGRFTISADTSKNTAYLQ | |
| | | | MNSLRAEDTAVYYCSRSFSRYGY | |
| | | | MDYWGQGTLVTVSSGGGASDYK | |
| | | | DDDDK | |
| HLAA3_9 | SWFSK | 92 | QQKPGKAPKLLIYSASFLYSGVPSR | 93 |
| | SAMDY | | FSGSRSGTDFTLTISSLQPEDFATY | |
| | | | YCQQSSHSPITFGQGTKVEIKRTGG | |
| | | | GSGGGGSGGGASEVQLVESGGGL | |
| | | | VQPGGSLRLSCAASGFNISTWLIH | |
| | | | WVRQAPGKGLEWVAVVYPDSSY | |
| | | | TYYADSVKGRFTISADTSKNTAYL | |
| | | | QMNSLRAEDTAVYYCSRSWFSKS | |
| | | | AMDYWGQGTLVTVSSGGGASDY | |
| | | | KDDDDK | |
| HLAA3_ | DMYSV | 94 | QQKPGKAPKLLIYSASFLYSGVPSR | 95 |
| 10 | RSAMD | | FSGSRSGTDFTLTISSLQPEDFATY | |
| | Y | | YCQQYYSYPITFGQGTKVEIKRTG | |
| | | | GGSGGGGSGGGASEVQLVESGGG | |
| | | | LVQPGGSLRLSCAASGFNVYYSG | |
| | | | MHWVRQAPGKGLEWVAKISPDSG | |
| | | | STYYADSVKGRFTISADTSKNTAY | |
| | | | LQMNSLRAEDTAVYYCSRDMYSV | |
| | | | RSAMDYWGQGTLVTVSSGGGAS | |
| | | | DYKDDDDK | |
| HLAA3_ | QYGYP | 96 | QQKPGKAPKLLIYSASFLYSGVPSR | 97 |
| 11 | YYFDY | | FSGSRSGTDFTLTISSLQPEDFATY | |
| | | | YCQQASSVPVTFGQGTKVEIKRTG | |
| | | | GGSGGGGSGGGASEVQLVESGGG | |
| | | | LVQPGGSLRLSCAASGFNIYSYGIH | |
| | | | WVRQAPGKGLEWVAKVSPSSGYT | |
| | | | YYADSVKGRFTISADTSKNTAYLQ | |
| | | | MNSLRAEDTAVYYCSRQYGYPYY | |
| | | | FDYWGQGTLVTVSSGGGASDYKD | |
| | | | DDDK | |

*The text in BOLD indicates the CDR-H3 location along the scFv.

TABLE C
Fraction of Selected Pools Corresponding to top 100 scFvs

| | FRACTION OF SELECTED POOLS | | | | |
|---|---|---|---|---|---|
| | CORRESPONDING TO TOP 100 SVFCS* | | | | |
| TARGET | ROUND 0 | ROUND 1 | ROUND 2 | ROUND 3 | ROUND 4 |
| RBD | 0.06% | 0.06% | 0.20% | 1.10% | 4.00% |
| S1 | 0.04% | 0.07% | 0.50% | 1.30% | 3.30% |
| FL | 0.04% | 0.05% | 0.08% | 0.30% | 1.10% |

*THE FRACTIONAL ABUNDANCE OF THE TOP 100 CLONES AS RANKED BY SBR OF THE ROUND 4 POOL WERE SUMMED FOR EACH ROUND FOR EACH TARGET.

TABLE D
Physical Characteristics of Potent Neutralizing mAbs

| Antibody | | | | K_{D} | | U- | |
|---|---|---|---|---|---|---|---|
| (Ligand) | Protein (Analyte) | kₐ (1/Ms) | k_{d} (1/s) | (nM) | Chi² | Value | RU |
| RBD_1 | SARS-CoV-2 FL | 0.840 × 10⁴ | 3.710 × 10⁻⁴ | 44.2 | 1.35 | 3 | ~30 |
| S1_13 | SARS-CoV-2 FL | 1.578 × 10⁴ | 1.840 × 10⁻⁴ | 11.7 | 0.168 | 1 | ~60 |
| FL_5 | SARS-CoV-2 FL | 1.146 × 10⁴ | 4.550 × 10⁻⁴ | 39.7 | 0.518 | 2 | ~20 |
| FL_10 | SARS-CoV-2 FL | 1.396 × 10⁴ | 1.048 × 10⁻⁴ | 7.5 | 1.34 | 3 | ~120 |
| FL_12 | SARS-CoV-2 FL | 1.489 × 10⁴ | 8.860 × 10⁻⁵ | 6 | 3.5 | 5 | ~120 |
| FL_13 | SARS-CoV-2 FL | 1.077 × 10⁴ | 1.022 × 10⁻⁴ | 9.5 | 1.23 | 4 | ~100 |

TABLE E
Coronavirus Antigens

| Name | Lineage | Antigen* | Mutations | Cat # |
|---|---|---|---|---|
| SARS- | — | RBD | — | 405920-V08H |
| CoV-2 | | | | (Sino Biological) |
| RBD | | | | |
| SARS- | — | S1 | — | 40591-V08H (Sino |
| CoV-2 S1 | | | | Biological) |
| SARS- | — | FL | — | 40589-V08B1 |
| CoV-2 FL | | (S1 + S2 | | (Sino Biological) |
| | | ECD) | | |
| MERS | — | RBD | — | 40071-V08B1 |
| RBD | | | | (Sino Biological) |
| MERS S1 | — | S1 | — | 40069-V08H (Sino |
| | | | | Biological) |
| MERS FL | — | FL | — | 40069-V08B (Sino |
| | | (S1 + S2 | | Biological) |
| | | ECD) | | |
| SARS- | B.1.1.7 | FL | HV69-70 deletion, | 40589-V08B6 |
| CoV-2 | | (S1 + S2 | Y144 deletion, N501Y, | (Sino Biological) |
| Alpha | | ECD) | A570D, D614G, | |
| | | | P681H, T716I, S982A, | |
| | | | D1118H | |
| SARS- | B.1.351 | S1 | K417N, E484K, | 40591-V08H10 |
| CoV-2 | | | N501Y, D614G | (Sino Biological) |
| Beta | | | | |
| SARS- | P.1 | RBD | K417T, E484K, | 40592-V08H86 |
| CoV-2 | | | N501Y | (Sino Biological) |
| Gamma | | | | |
| SARS- | B.1.617/ | RBD | L452R, E484Q | 40592-V08H88 |
| CoV-2 | B.1.617.1/ | | | (Sino Biological) |
| Delta | B.1.617.3 | | | |
| SARS- | — | RBD | G476S | 40592-V08H8 |
| CoV-2 | | | | (Sino Biological) |
| G476S | | | | |
| SARS- | — | RBD | V483A | 40592-V08H5 |
| CoV-2 | | | | (Sino Biological) |
| V483A | | | | |
| SARS- | — | RBD | N439K | 40592-V08H14 |
| CoV-2 | | | | (Sino Biological) |
| N439K | | | | |
| SARS- | Mink | RBD | Y453F | 40592-V08H80 |
| CoV-2 | variant | | | (Sino Biological) |
| Y453F | | | | |
| SARS- | — | RBD | N501Y | 40592-V08H82 |
| CoV-2 | | | | (Sino Biological) |
| N501Y | | | | |
| SARS- | — | S1 | D614G | 40591-V08H3 |
| CoV-2 | | | | (Sino Biological) |
| D614G | | | | |
| SARS- | — | RBD | — | 40150-V08B2 |
| CoV RBD | | | | (Sino Biological) |
| SARS- | — | S1 | — | 40150-V08B1 |
| CoV S1 | | | | (Sino Biological) |
| SARS- | — | FL | — | 40634-V08B (Sino |
| CoV FL | | (S1 + S2 | | Biological) |
| | | ECD) | | |
| SARS- | B.1.351 | S1 | L18F, D80A, D215G, | 40591-V08H15 |
| CoV-2 | | | LAL242-244 deletion, | (Sino Biological) |
| Beta-2 | | | R246I, K417N, | |
| | | | E484K, N501Y, | |
| | | | D614G | |
| SARS- | B.1.617.2 | RBD | L452R,T478K | 40592-V08H90 |
| CoV-2 | | | | (Sino Biological) |
| Delta-2 | | | | |
| SARS- | B.1.351 | RBD | E484K | 40592-V08H84 |
| CoV-2 | | | | (Sino Biological) |
| E484K | | | | |
| SARS- | | RBD | | 40150-V08B2 |
| CoV | | | | (Sino Biological) |
| SARS- | | FL | | 40634-V08B (Sino |
| CoV | | (S1 + S2 | | Biological) |
| | | ECD) | | |

*All antigens were His-tagged.

TABLE F
Sequences of SARS-CoV-2 scFvs

| | | SEQ | | SEQ | | SEQ |
|---|---|---|---|---|---|---|
| | | ID | | ID | | ID |
| Clone | CDR-H3 | NO: | Amino Acid Sequence of scFv* | NO: | Nucleotide Sequence of scFv | NO: |
| RBD_1 | DKSTYES | 98 | KPGKAPKLLIYSASFLYSGVPSRFSG | 99 | AAACCGGGTAAAGCACCGAAACTGCTGAT | 100 |
| | AFDY | | SRSGTDFTLTISSLQPEDFATYYCQQ | | TTATAGCGCCTCTTTCCTGTATAGCGGTGT | |
| | | | SFYSPFTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNISNASMHWVRQAP | | GCAGCCGGAAGATTTTGCAACCTATTATTG | |
| | | | GKGLEWVAWIYGSGDYTYYADSV | | CCAGCAA TCTTTTTACTCTCCTTTTACCTTT | |
| | | | KGRFTISADTSKNTAYLQMNSLRAE | | GGCCAGGGCACCAAAGTTGAAATTAAACG | |
| | | | DTAVYYCSRDKSTYESAFDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTAACGCATCTATGC | |
| | | | | | ATTGGGTGCGCCAGGCACCTGGTAAAGGTC | |
| | | | | | TGGAATGGGTTGCCTGGATCTACGGTTCTG | |
| | | | | | GTGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGATAAATCTACCTACGAA | |
| | | | | | TCTGCATTTGATTACTGGGGTCAGGGCACC | |
| RBD_2 | GGVHYG | 101 | KPGKAPKLLIYSASFLYSGVPSRFSG | 102 | AAACCGGGTAAAGCACCGAAACTGCTGAT | 103 |
| | AMDY | | SRSGTDFTLTISSLQPEDFATYYCQQ | | TTATAGCGCCTCTTTCCTGTATAGCGGTGT | |
| | | | YDSPITFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNIDYKFMHWVRQAP | | GCAGCCGGAAGATTTTGCAACCTATTATTG | |
| | | | GKGLEWVALFGPDQDYTYYADSV | | CCAGCAA TACGATTCTCCTATCACCTTTGG | |
| | | | KGRFTISADTSKNTAYLQMNSLRAE | | CCAGGGCACCAAAGTTGAAATTAAACGTA | |
| | | | DTAVYYCSRGGVHYGAMDYWGQ | | CCGGTGGTGGTTCAGGTGGCGGTGGCAGC | |
| | | | GT | | GGAGGCGGAGCCAGCGAAGTGCAGCTGGT | |
| | | | | | TGAAAGCGGTGGCGGTCTGGTTCAGCCAG | |
| | | | | | GTGGTAGCCTGCGTCTGAGCTGTGCAGCAA | |
| | | | | | GCGGTTTTAACATCGATTACAAATTTATGC | |
| | | | | | ATTGGGTGCGCCAGGCACCTGGTAAAGGTC | |
| | | | | | TGGAATGGGTTGCCCTGTTTGGTCCAGATC | |
| | | | | | AGGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTGGTGTTCATTACGGT | |
| | | | | | GCAATGGATTACTGGGGTCAGGGCACC | |
| RBD_3 | QSSYAY | 104 | KPGKAPKLLIYSASFLYSGVPSRFSG | 105 | AAACCGGGTAAAGCACCGAAACTGCTGAT | 106 |
| | MDY | | SRSGTDFTLTISSLQPEDFATYYCQQ | | TTATAGCGCCTCTTTCCTGTATAGCGGTGT | |
| | | | QWYSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLADSHIHWVRQAP | | GCAGCCGGAAGATTTTGCAACCTATTATTG | |
| | | | GKGLEWVAQLYGDSDYTNYADSV | | CCAGCAA CAGTGGTACTCTCCTATCACCTT | |
| | | | KGRFTISADTSKNTAYLQMNSLRAE | | TGGCCAGGGCACCAAAGTTGAAATTAAAC | |
| | | | DTAVYYCSRQSSYAYMDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGGCAGATTCTCATATC | |
| | | | | | CATTGGGTGCGCCAGGCACCTGGTAAAGGT | |
| | | | | | CTGGAATGGGTTGCCCAGCTGTACGGTGAT | |
| | | | | | TCTGATTACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCCAGTCTTCTTACGCATA | |
| | | | | | CATGGATTACTGGGGTCAGGGCACC | |
| RBD_4 | NYSSYAF | 107 | KPGKAPKLLIYSASFLYSGVPSRFSG | 108 | AAACCGGGTAAAGCACCGAAACTGCTGAT | 109 |
| | DY | | SRSGTDFTLTISSLQPEDFATYYCQQ | | TTATAGCGCCTCTTTCCTGTATAGCGGTGT | |
| | | | PYSYPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVYSYQMHWVRQA | | GCAGCCGGAAGATTTTGCAACCTATTATTG | |
| | | | PGKGLEWVAYVSGYSGNTYYADS | | CCAGCAA CCATACTCTTACCCTGTTACCTT | |
| | | | VKGRFTISADTSKNTAYLQMNSLRA | | TGGCCAGGGCACCAAAGTTGAAATTAAAC | |
| | | | EDTAVYYCSRNYSSYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTTACTCTTACCAGATG | |
| | | | | | CATTGGGTGCGCCAGGCACCTGGTAAAGGT | |
| | | | | | CTGGAATGGGTTGCCTACGTTTCTGGTTAC | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCAACTACTCTTCTTACGC | |
| | | | | | ATTTGATTACTGGGGTCAGGGCACC | |
| RBD_5 | NYGGYA | 110 | KPGKAPKLLIYSASFLYSGVPSRFSG | 111 | AAACCGGGTAAAGCACCGAAACTGCTGAT | 112 |
| | FDY | | SRSGTDFTLTISSLQPEDFATYYCQQ | | TTATAGCGCCTCTTTCCTGTATAGCGGTGT | |
| | | | SSPPPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLYSYDIHWVRQAP | | GCAGCCGGAAGATTTTGCAACCTATTATTG | |
| | | | GKGLEWVAQVVPESGNTYYADSV | | CCAGCAA TCTTCTCCACCACCTGTTACCTT | |
| | | | KGRFTISADTSKNTAYLQMNSLRAE | | TGGCCAGGGCACCAAAGTTGAAATTAAAC | |
| | | | DTAVYYCSRNYGGYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTACTCTTACGATATC | |
| | | | | | CATTGGGTGCGCCAGGCACCTGGTAAAGGT | |
| | | | | | CTGGAATGGGTTGCCCAGGTTGTTCCAGAA | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCAACTACGGTGGTTACGC | |
| | | | | | ATTTGATTACTGGGGTCAGGGCACC | |
| RBD_6 | DMYYSS | 113 | KPGKAPKLLIYSASFLYSGVPSRFSG | 114 | AAACCGGGTAAAGCACCGAAACTGCTGAT | 115 |
| | YAVDV | | SRSGTDFTLTISSLQPEDFATYYCQQ | | TTATAGCGCCTCTTTCCTGTATAGCGGTGT | |
| | | | YYEPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLSSSGMHWVRQA | | GCAGCCGGAAGATTTTGCAACCTATTATTG | |
| | | | PGKGLEWVALFDPLYDNTMYADSV | | CCAGCAA TACTACGAACCTGTTACCTTTGG | |
| | | | KGRFTISADTSKNTAYLQMNSLRAE | | CCAGGGCACCAAAGTTGAAATTAAACGTA | |
| | | | DTAVYYCSRDMYYSSYAVDVWGQ | | CCGGTGGTGGTTCAGGTGGCGGTGGCAGC | |
| | | | GT | | GGAGGCGGAGCCAGCGAAGTGCAGCTGGT | |
| | | | | | TGAAAGCGGTGGCGGTCTGGTTCAGCCAG | |
| | | | | | GTGGTAGCCTGCGTCTGAGCTGTGCAGCAA | |
| | | | | | GCGGTTTTAACCTGTCTTCTTCTGGTATGCA | |
| | | | | | TTGGGTGCGCCAGGCACCTGGTAAAGGTCT | |
| | | | | | GGAATGGGTTGCCCTGTTTGATCCACTGTA | |
| | | | | | CGATAACACCATGTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGATATGTACTACTCTTCTT | |
| | | | | | ACGCAGTTGATGTTTGGGGTCAGGGCACC | |
| RBD_7 | AEYYST | 116 | KPGKAPKLLIYSASFLYSGVPSRFSG | 117 | AAACCGGGTAAAGCACCGAAACTGCTGAT | 118 |
| | AFDY | | SRSGTDFTLTISSLQPEDFATYYCQQ | | TTATAGCGCCTCTTTCCTGTATAGCGGTGT | |
| | | | SFVYPHTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVYSYSMHWVRQA | | GCAGCCGGAAGATTTTGCAACCTATTATTG | |
| | | | PGKGLEWVAAINYDGSYTYYADSV | | CCAGCAA TCTTTTGTTTACCCTCATACCTT | |
| | | | KGRFTISADTSKNTAYLQMNSLRAE | | TGGCCAGGGCACCAAAGTTGAAATTAAAC | |
| | | | DTAVYYCSRAEYYSTAFDYWGQG | | GTACCGGTGGTGGTTCAGGTGGCGGTGGCA | |
| | | | T | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTTACTCTTACTCTATGC | |
| | | | | | ATTGGGTGCGCCAGGCACCTGGTAAAGGTC | |
| | | | | | TGGAATGGGTTGCCGCAATCAACTACGATG | |
| | | | | | GTTCTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAGAATACTACTCTACC | |
| | | | | | GCATTTGATTACTGGGGTCAGGGCACC | |
| RBD_8 | SDSYAFD | 119 | KPGKAPKLLIYSASFLYSGVPSRFSG | 120 | AAACCGGGTAAAGCACCGAAACTGCTGAT | 121 |
| | Y | | SRSGTDFTLTISSLQPEDFATYYCQQ | | TTATAGCGCCTCTTTCCTGTATAGCGGTGT | |
| | | | YYWSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVTYGYMHWVRQ | | GCAGCCGGAAGATTTTGCAACCTATTATTG | |
| | | | APGKGLEWVAWLLGDYGYTYYAD | | CCAGCAA TACTACTGGTCTCCTATCACCTT | |
| | | | SVKGRFTISADTSKNTAYLQMNSLR | | TGGCCAGGGCACCAAAGTTGAAATTAAAC | |
| | | | AEDTAVYYCSRSDSYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTACCTACGGTTACATG | |
| | | | | | CATTGGGTGCGCCAGGCACCTGGTAAAGGT | |
| | | | | | CTGGAATGGGTTGCCTGGCTGCTGGGTGAT | |
| | | | | | TACGGTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTCTGATTCTTACGCATTT | |
| | | | | | GATTACTGGGGTCAGGGCACC | |
| RBD_9 | YATYAF | 122 | KPGKAPKLLIYSASFLYSGVPSRFSG | 123 | AAACCGGGTAAAGCACCGAAACTGCTGAT | 124 |
| | DY | | SRSGTDFTLTISSLQPEDFATYYCQQ | | TTATAGCGCCTCTTTCCTGTATAGCGGTGT | |
| | | | SFKTPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNISYHGMHWVRQA | | GCAGCCGGAAGATTTTGCAACCTATTATTG | |
| | | | PGKGLEWVAVIQPSGGYTYYADSV | | CCAGCAA TCTTTTAAAACCCCTGTTACCTT | |
| | | | KGRFTISADTSKNTAYLQMNSLRAE | | TGGCCAGGGCACCAAAGTTGAAATTAAAC | |
| | | | DTAVYYCSRYATYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTTACCATGGTATG | |
| | | | | | CATTGGGTGCGCCAGGCACCTGGTAAAGGT | |
| | | | | | CTGGAATGGGTTGCCGTTATCCAGCCATCT | |
| | | | | | GGTGGTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACGCAACCTACGCATT | |
| | | | | | TGATTACTGGGGTCAGGGCACC | |
| RBD_ | GHSYGY | 125 | KPGKAPKLLIYSASFLYSGVPSRFSG | 126 | AAACCGGGTAAAGCACCGAAACTGCTGAT | 127 |
| 10 | ALAY | | SRSGTDFTLTISSLQPEDFATYYCQQ | | TTATAGCGCCTCTTTCCTGTATAGCGGTGT | |
| | | | SAYGPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIYHESMHWVRQA | | GCAGCCGGAAGATTTTGCAACCTATTATTG | |
| | | | PGKGLEWVAEIRPMTGFTYYADSV | | CCAGCAA TCTGCATACGGTCCTATCACCTT | |
| | | | KGRFTISADTSKNTAYLQMNSLRAE | | TGGCCAGGGCACCAAAGTTGAAATTAAAC | |
| | | | DTAVYYCSRGHSYGYALAYWGQG | | GTACCGGTGGTGGTTCAGGTGGCGGTGGCA | |
| | | | T | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACCATGAATCTATG | |
| | | | | | CATTGGGTGCGCCAGGCACCTGGTAAAGGT | |
| | | | | | CTGGAATGGGTTGCCGAAATCAGACCAAT | |
| | | | | | GACCGGTTTTACCTACTATGCCGATAGCGT | |
| | | | | | GAAAGGTCGCTTTACCATTAGCGCAGATAC | |
| | | | | | CAGCAAAAATACCGCATACCTGCAGATGA | |
| | | | | | ATAGTCTGCGTGCAGAGGATACAGCAGTGT | |
| | | | | | ATTATTGTAGCCGCGGTCATTCTTACGGTT | |
| | | | | | ACGCACTGGCATACTGGGGTCAGGGCACC | |
| RBD_ | GNGSAF | 128 | KPGKAPKLLIYSASFLYSGVPSRFSG | 129 | AAACCGGGTAAAGCACCGAAACTGCTGAT | 130 |
| 11 | DY | | SRSGTDFTLTISSLQPEDFATYYCQQ | | TTATAGCGCCTCTTTCCTGTATAGCGGTGT | |
| | | | AYGVPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLSYDPMHWVRQA | | GCAGCCGGAAGATTTTGCAACCTATTATTG | |
| | | | PGKGLEWVAHIEPWSGNTYYADSV | | CCAGCAA GCATACGGTGTTCCTATCACCTT | |
| | | | KGRFTISADTSKNTAYLQMNSLRAE | | TGGCCAGGGCACCAAAGTTGAAATTAAAC | |
| | | | DTAVYYCSRGNGSAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTCTTACGATCCTATG | |
| | | | | | CATTGGGTGCGCCAGGCACCTGGTAAAGGT | |
| | | | | | CTGGAATGGGTTGCCCATATCGAACCATGG | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGGTAACGGTTCTGCATT | |
| | | | | | TGATTACTGGGGTCAGGGCACC | |
| RBD_ | GLLHHS | 131 | KPGKAPKLLIYSASFLYSGVPSRFSG | 132 | AAACCGGGTAAAGCACCGAAACTGCTGAT | 133 |
| 12 | SGVALD | | SRSGTDFTLTISSLQPEDFATYYCQQ | | TTATAGCGCCTCTTTCCTGTATAGCGGTGT | |
| | Y | | SYYYPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLYNSDMHWVRQA | | GCAGCCGGAAGATTTTGCAACCTATTATTG | |
| | | | PGKGLEWVAIIYPGSGNTYYADSVK | | CCAGCAA TCTTACTACTACCCTGTTACCTT | |
| | | | GRFTISADTSKNTAYLQMNSLRAED | | TGGCCAGGGCACCAAAGTTGAAATTAAAC | |
| | | | TAVYYCSRGLLHHSSGVALDYWG | | GTACCGGTGGTGGTTCAGGTGGCGGTGGCA | |
| | | | QGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTACAACTCTGATATG | |
| | | | | | CATTGGGTGCGCCAGGCACCTGGTAAAGGT | |
| | | | | | CTGGAATGGGTTGCCATCATCTACCCAGGT | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGGTCTGCTGCATCATTCT | |
| | | | | | TCTGGTGTTGCACTGGATTACTGGGGTCAG | |
| | | | | | GGCACC | |
| RBD_ | WERYSIS | 134 | KPGKAPKLLIYSASFLYSGVPSRFSG | 135 | AAACCGGGTAAAGCACCGAAACTGCTGAT | 136 |
| 13 | MDY | | SRSGTDFTLTISSLQPEDFATYYCQQ | | TTATAGCGCCTCTTTCCTGTATAGCGGTGT | |
| | | | SSSYPITFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNIYQYDIHWVRQAPG | | GCAGCCGGAAGATTTTGCAACCTATTATTG | |
| | | | KGLEWVASLSDSSGSTNYADSVKG | | CCAGCAA TCTTCTTCTTACCCTATCACCTT | |
| | | | RFTISADTSKNTAYLQMNSLRAEDT | | TGGCCAGGGCACCAAAGTTGAAATTAAAC | |
| | | | AVYYCSRWERYSISMDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACCAGTACGATATC | |
| | | | | | CATTGGGTGCGCCAGGCACCTGGTAAAGGT | |
| | | | | | CTGGAATGGGTTGCCTCTCTGTCTGATTCTT | |
| | | | | | CTGGTTCTACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTGGGAAAGATACTCTATC | |
| | | | | | TCTATGGATTACTGGGGTCAGGGCACC | |
| S1_1 | ADWYSL | 137 | KPGKAPKLLIYSASFLYSGVPSRFSG | 138 | AAACCGGGTAAAGCACCGAAACTGCTGAT | 139 |
| | SEFYYS | | SRSGTDFTLTISSLQPEDFATYYCQQ | | TTATAGCGCCTCTTTCCTGTATAGCGGTGT | |
| | MDY | | SSAAPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLGQYDIHWVRQA | | GCAGCCGGAAGATTTTGCAACCTATTATTG | |
| | | | PGKGLEWVAQFMYRSGYTNYADS | | CCAGCAA TCTTCTGCAGCACCTATCACCTT | |
| | | | VKGRFTISADTSKNTAYLQMNSLRA | | TGGCCAGGGCACCAAAGTTGAAATTAAAC | |
| | | | EDTAVYYCSRADWYSLSEFYYSM | | GTACCGGTGGTGGTTCAGGTGGCGGTGGCA | |
| | | | DYWGQGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGGGTCAGTACGATATC | |
| | | | | | CATTGGGTGCGCCAGGCACCTGGTAAAGGT | |
| | | | | | CTGGAATGGGTTGCCCAGTTTATGTACAGA | |
| | | | | | TCTGGTTACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGCAGATTGGTACTCTCT | |
| | | | | | GTCTGAATTTTACTACTCTATGGATTACTG | |
| | | | | | GGGTCAGGGCACC | |
| S1_2 | KMSSAY | 140 | KPGKAPKLLIYSASFLYSGVPSRFSG | 141 | AAACCGGGTAAAGCACCGAAACTGCTGAT | 142 |
| | ALDY | | SRSGTDFTLTISSLQPEDFATYYCQQ | | TTATAGCGCCTCTTTCCTGTATAGCGGTGT | |
| | | | SYYLPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNFSYEQMHWVRQA | | GCAGCCGGAAGATTTTGCAACCTATTATTG | |
| | | | PGKGLEWVADFNSGQSNTYYADSV | | CCAGCAA TCTTACTACCTGCCTATCACCTT | |
| | | | KGRFTISADTSKNTAYLQMNSLRAE | | TGGCCAGGGCACCAAAGTTGAAATTAAAC | |
| | | | DTAVYYCSRKMSSAYALDYWGQG | | GTACCGGTGGTGGTTCAGGTGGCGGTGGCA | |
| | | | T | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACTTTTCTTACGAACAGATG | |
| | | | | | CATTGGGTGCGCCAGGCACCTGGTAAAGGT | |
| | | | | | CTGGAATGGGTTGCCGATTTTAACTCTGGT | |
| | | | | | CAGTCTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCAAAATGTCTTCTGCATA | |
| | | | | | CGCACTGGATTACTGGGGTCAGGGCACC | |
| S1_3 | NDLSSY | 143 | KPGKAPKLLIYSASFLYSGVPSRFSG | 144 | AAACCGGGTAAAGCACCGAAACTGCTGAT | 145 |
| | HYAMDY | | SRSGTDFTLTISSLQPEDFATYYCQQ | | TTATAGCGCCTCTTTCCTGTATAGCGGTGT | |
| | | | GTFSSPLTFGQGTKVEIKRTGGGSG | | GCCGAGCCGTTTTAGCGGTAGCCGTAGCGG | |
| | | | GGGSGGGASEVQLVESGGGLVQPG | | CACCGATTTTACCCTGACCATTAGCAGCCT | |
| | | | GSLRLSCAASGFNFTMYSMHWVRQ | | GCAGCCGGAAGATTTTGCAACCTATTATTG | |
| | | | APGKGLEWVATSWGDSGYTYYAD | | CCAGCAA GGTACCTTTTCTTCTCCTCTGAC | |
| | | | SVKGRFTISADTSKNTAYLQMNSLR | | CTTTGGCCAGGGCACCAAAGTTGAAATTAA | |
| | | | AEDTAVYYCSRNDLSSYHYAMDY | | ACGTACCGGTGGTGGTTCAGGTGGCGGTGG | |
| | | | WGQGT | | CAGCGGAGGCGGAGCCAGCGAAGTGCAGC | |
| | | | | | TGGTTGAAAGCGGTGGCGGTCTGGTTCAGC | |
| | | | | | CAGGTGGTAGCCTGCGTCTGAGCTGTGCAG | |
| | | | | | CAAGCGGTTTTAACTTTACCATGTACTCTA | |
| | | | | | TGCATTGGGTGCGCCAGGCACCTGGTAAAG | |
| | | | | | GTCTGGAATGGGTTGCCACCTCTTGGGGTG | |
| | | | | | ATTCTGGTTACACCTACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCAACGATCTGTCTTC | |
| | | | | | TTACCATTACGCAATGGATTACTGGGGTCA | |
| | | | | | GGGCACC | |
| S1_4 | EYQKYS | 146 | KPGKAPKLLIYSASFLYSGVPSRFSG | 147 | AAACCGGGTAAAGCACCGAAACTGCTGAT | 148 |
| | FDY | | SRSGTDFTLTISSLQPEDFATYYCQQ | | TTATAGCGCCTCTTTCCTGTATAGCGGTGT | |
| | | | PYYFPDTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIGYSVMHWVRQA | | GCAGCCGGAAGATTTTGCAACCTATTATTG | |
| | | | PGKGLEWVAVVGPGSGNTNYADS | | CCAGCAA CCATACTACTTTCCTGATACCTT | |
| | | | VKGRFTISADTSKNTAYLQMNSLRA | | TGGCCAGGGCACCAAAGTTGAAATTAAAC | |
| | | | EDTAVYYCSREYQKYSFDYWGQG | | GTACCGGTGGTGGTTCAGGTGGCGGTGGCA | |
| | | | T | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCGGTTACTCTGTTATG | |
| | | | | | CATTGGGTGCGCCAGGCACCTGGTAAAGGT | |
| | | | | | CTGGAATGGGTTGCCGTTGTTGGTCCAGGC | |
| | | | | | TCTGGTAACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGAATACCAGAAATACTC | |
| | | | | | TTTTGATTACTGGGGTCAGGGCACC | |
| S1_5 | AIYNISS | 149 | KPGKAPKLLIYSASFLYSGVPSRFSG | 150 | AAACCGGGTAAAGCACCGAAACTGCTGAT | 151 |
| | ASYYTY | | SRSGTDFTLTISSLQPEDFATYYCQQ | | TTATAGCGCCTCTTTCCTGTATAGCGGTGT | |
| | ADGFDY | | YSAYPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVSAIAIHWVRQAP | | GCAGCCGGAAGATTTTGCAACCTATTATTG | |
| | | | GKGLEWVATIYPGSSYTYYADSVK | | CCAGCAA TACTCTGCATACCCTGTTACCTT | |
| | | | GRFTISADTSKNTAYLQMNSLRAED | | TGGCCAGGGCACCAAAGTTGAAATTAAAC | |
| | | | TAVYYCSRAIYNISSASYYTYADGF | | GTACCGGTGGTGGTTCAGGTGGCGGTGGCA | |
| | | | DYWGQGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTTCTGCAATCGCAATC | |
| | | | | | CATTGGGTGCGCCAGGCACCTGGTAAAGGT | |
| | | | | | CTGGAATGGGTTGCCACCATCTACCCAGGT | |
| | | | | | TCTTCTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGCAATCTACAACATCTC | |
| | | | | | TTCTGCATCTTACTACACCTACGCAGATGG | |
| | | | | | TTTTGATTACTGGGGTCAGGGCACC | |
| S1_6 | ASYSSYF | 152 | KPGKAPKLLIYSASFLYSGVPSRFSG | 153 | AAACCGGGTAAAGCACCGAAACTGCTGAT | 154 |
| | DY | | SRSGTDFTLTISSLQPEDFATYYCQQ | | TTATAGCGCCTCTTTCCTGTATAGCGGTGT | |
| | | | YGRPITFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNIVYQSMHWVRQAP | | GCAGCCGGAAGATTTTGCAACCTATTATTG | |
| | | | GKGLEWVANLSYDGSYTNYADSV | | CCAGCAA TACGGTAGACCTATCACCTTTG | |
| | | | KGRFTISADTSKNTAYLQMNSLRAE | | GCCAGGGCACCAAAGTTGAAATTAAACGT | |
| | | | DTAVYYCSRASYSSYFDYWGQGT | | ACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCGTTTACCAGTCTATGC | |
| | | | | | ATTGGGTGCGCCAGGCACCTGGTAAAGGTC | |
| | | | | | TGGAATGGGTTGCCAACCTGTCTTACGATG | |
| | | | | | GTTCTTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCATCTTACTCTTCTTACT | |
| | | | | | TTGATTACTGGGGTCAGGGCACC | |
| S1_7 | WSSSAF | 155 | KPGKAPKLLIYSASFLYSGVPSRFSG | 156 | AAACCGGGTAAAGCACCGAAACTGCTGAT | 157 |
| | DY | | SRSGTDFTLTISSLQPEDFATYYCQQ | | TTATAGCGCCTCTTTCCTGTATAGCGGTGT | |
| | | | GVYGPVTFGQGTKVEIKRTGGGSG | | GCCGAGCCGTTTTAGCGGTAGCCGTAGCGG | |
| | | | GGGSGGGASEVQLVESGGGLVQPG | | CACCGATTTTACCCTGACCATTAGCAGCCT | |
| | | | GSLRLSCAASGFNVSYYYMHWVRQ | | GCAGCCGGAAGATTTTGCAACCTATTATTG | |
| | | | APGKGLEWVAFLNPDSGNTYYADS | | CCAGCAA GGTGTTTACGGTCCTGTTACCTT | |
| | | | VKGRFTISADTSKNTAYLQMNSLRA | | TGGCCAGGGCACCAAAGTTGAAATTAAAC | |
| | | | EDTAVYYCSRWSSSAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTTCTTACTACTACATG | |
| | | | | | CATTGGGTGCGCCAGGCACCTGGTAAAGGT | |
| | | | | | CTGGAATGGGTTGCCTTTCTGAACCCAGAT | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTGGTCTTCTTCTGCATTT | |
| | | | | | GATTACTGGGGTCAGGGCACC | |
| S1_8 | GRTDYS | 158 | KPGKAPKLLIYSASFLYSGVPSRFSG | 159 | AAACCGGGTAAAGCACCGAAACTGCTGAT | 160 |
| | SYYYSY | | SRSGTDFTLTISSLQPEDFATYYCQQ | | TTATAGCGCCTCTTTCCTGTATAGCGGTGT | |
| | AFDY | | GSSSPSTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVSSTYMHWVRQA | | GCAGCCGGAAGATTTTGCAACCTATTATTG | |
| | | | PGKGLEWVAQIYPHSGPTFYADSV | | CCAGCAA GGTTCTTCTTCTCCTTCTACCTTT | |
| | | | KGRFTISADTSKNTAYLQMNSLRAE | | GGCCAGGGCACCAAAGTTGAAATTAAACG | |
| | | | DTAVYYCSRGRTDYSSYYYSYAFD | | TACCGGTGGTGGTTCAGGTGGCGGTGGCAG | |
| | | | YWGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTTCTACCTACATGCA | |
| | | | | | TTGGGTGCGCCAGGCACCTGGTAAAGGTCT | |
| | | | | | GGAATGGGTTGCCCAGATCTACCCACATTC | |
| | | | | | TGGTCCAACCTTTTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGGTAGAACCGATTACTCTT | |
| | | | | | CTTACTACTACTCTTACGCATTTGATTACTG | |
| | | | | | GGGTCAGGGCACC | |
| S1_9 | GNGYYA | 161 | KPGKAPKLLIYSASFLYSGVPSRFSG | 162 | AAACCGGGTAAAGCACCGAAACTGCTGAT | 163 |
| | FAMDY | | SRSGTDFTLTISSLQPEDFATYYCQQ | | TTATAGCGCCTCTTTCCTGTATAGCGGTGT | |
| | | | YSYYPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLSYSYIHWVRQAP | | GCAGCCGGAAGATTTTGCAACCTATTATTG | |
| | | | GKGLEWVADLYPYGGPTNYADSV | | CCAGCAA TACTCTTACTACCCTATCACCTT | |
| | | | KGRFTISADTSKNTAYLQMNSLRAE | | TGGCCAGGGCACCAAAGTTGAAATTAAAC | |
| | | | DTAVYYCSRGNGYYAFAMDYWG | | GTACCGGTGGTGGTTCAGGTGGCGGTGGCA | |
| | | | QGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTCTTACTCTTACATCC | |
| | | | | | ATTGGGTGCGCCAGGCACCTGGTAAAGGTC | |
| | | | | | TGGAATGGGTTGCCGATCTGTACCCATACG | |
| | | | | | GTGGTCCAACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTAACGGTTACTACGCA | |
| | | | | | TTTGCAATGGATTACTGGGGTCAGGGCACC | |
| S1_10 | DMYYSS | 164 | KPGKAPKLLIYSASFLYSGVPSRFSG | 165 | AAACCGGGTAAAGCACCGAAACTGCTGAT | 166 |
| | YYMDY | | SRSGTDFTLTISSLQPEDFATYYCQQ | | TTATAGCGCCTCTTTCCTGTATAGCGGTGT | |
| | | | YWYSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNISASGIHWVRQAP | | GCAGCCGGAAGATTTTGCAACCTATTATTG | |
| | | | GKGLEWVAWIADLSGGTSYADSVK | | CCAGCAA TACTGGTACTCTCCTATCACCTT | |
| | | | GRFTISADTSKNTAYLQMNSLRAED | | TGGCCAGGGCACCAAAGTTGAAATTAAAC | |
| | | | TAVYYCSRDMYYSSYYMDYWGQ | | GTACCGGTGGTGGTTCAGGTGGCGGTGGCA | |
| | | | GT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTGCATCTGGTATC | |
| | | | | | CATTGGGTGCGCCAGGCACCTGGTAAAGGT | |
| | | | | | CTGGAATGGGTTGCCTGGATCGCAGATCTG | |
| | | | | | TCTGGTGGTACCTCTTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGATATGTACTACTCTTCT | |
| | | | | | TACTACATGGATTACTGGGGTCAGGGCACC | |
| S1_11 | NYKAYA | 167 | KPGKAPKLLIYSASFLYSGVPSRFSG | 168 | AAACCGGGTAAAGCACCGAAACTGCTGAT | 169 |
| | FDY | | SRSGTDFTLTISSLQPEDFATYYCQQ | | TTATAGCGCCTCTTTCCTGTATAGCGGTGT | |
| | | | SYIPPTFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNIYSYSIHWVRQAPG | | GCAGCCGGAAGATTTTGCAACCTATTATTG | |
| | | | KGLEWVATIPPDSDATYYADSVKG | | CCAGCAA TCTTACATCCCTCCAACCTTTGG | |
| | | | RFTISADTSKNTAYLQMNSLRAEDT | | CCAGGGCACCAAAGTTGAAATTAAACGTA | |
| | | | AVYYCSRNYKAYAFDYWGQGT | | CCGGTGGTGGTTCAGGTGGCGGTGGCAGC | |
| | | | | | GGAGGCGGAGCCAGCGAAGTGCAGCTGGT | |
| | | | | | TGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACTCTTACTCTATCCA | |
| | | | | | TTGGGTGCGCCAGGCACCTGGTAAAGGTCT | |
| | | | | | GGAATGGGTTGCCACCATCCCACCAGATTC | |
| | | | | | TGATGCAACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCAACTACAAAGCATACGCAT | |
| | | | | | TTGATTACTGGGGTCAGGGCACC | |
| S1_12 | YSYDAM | 170 | KPGKAPKLLIYSASFLYSGVPSRFSG | 171 | AAACCGGGTAAAGCACCGAAACTGCTGAT | 172 |
| | DY | | SRSGTDFTLTISSLQPEDFATYYCQQ | | TTATAGCGCCTCTTTCCTGTATAGCGGTGT | |
| | | | ISVTPITFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNISHQSMHWVRQAP | | GCAGCCGGAAGATTTTGCAACCTATTATTG | |
| | | | GKGLEWVAYVYPDSDYTEYADSV | | CCAGCAA ATCTCTGTTACCCCTATCACCTT | |
| | | | KGRFTISADTSKNTAYLQMNSLRAE | | TGGCCAGGGCACCAAAGTTGAAATTAAAC | |
| | | | DTAVYYCSRYSYDAMDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTCATCAGTCTATG | |
| | | | | | CATTGGGTGCGCCAGGCACCTGGTAAAGGT | |
| | | | | | CTGGAATGGGTTGCCTACGTTTACCCAGAT | |
| | | | | | TCTGATTACACCGAATATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACTCTTACGATGCAAT | |
| | | | | | GGATTACTGGGGTCAGGGCACC | |
| S1_13 | YHYMVS | 173 | KPGKAPKLLIYSASFLYSGVPSRFSG | 174 | AAACCGGGTAAAGCACCGAAACTGCTGAT | 175 |
| | FDY | | SRSGTDFTLTISSLQPEDFATYYCQQ | | TTATAGCGCCTCTTTCCTGTATAGCGGTGT | |
| | | | YSSAPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIWSWVMHWVRQ | | GCAGCCGGAAGATTTTGCAACCTATTATTG | |
| | | | APGKGLEWVALFHPDSGNTYYADS | | CCAGCAA TACTCTTCTGCACCTATCACCTT | |
| | | | VKGRFTISADTSKNTAYLQMNSLRA | | TGGCCAGGGCACCAAAGTTGAAATTAAAC | |
| | | | EDTAVYYCSRYHYMVSFDYWGQG | | GTACCGGTGGTGGTTCAGGTGGCGGTGGCA | |
| | | | T | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTGGTCTTGGGTTATG | |
| | | | | | CATTGGGTGCGCCAGGCACCTGGTAAAGGT | |
| | | | | | CTGGAATGGGTTGCCCTGTTTCATCCAGAT | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACCATTACATGGTTTCT | |
| | | | | | TTTGATTACTGGGGTCAGGGCACC | |
| FL_1 | SYYQYA | 176 | KPGKAPKLLIYSASFLYSGVPSRFSG | 177 | AAACCGGGTAAAGCACCGAAACTGCTGAT | 178 |
| | FDV | | SRSGTDFTLTISSLQPEDFATYYCQQ | | TTATAGCGCCTCTTTCCTGTATAGCGGTGT | |
| | | | TYTPPRTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIYSYSIHWVRQAP | | GCAGCCGGAAGATTTTGCAACCTATTATTG | |
| | | | GKGLEWVAEFSDQSGYTYYADSVK | | CCAGCAA ACCTACACCCCACCTAGAACCT | |
| | | | GRFTISADTSKNTAYLQMNSLRAED | | TTGGCCAGGGCACCAAAGTTGAAATTAAA | |
| | | | TAVYYCSRSYYQYAFDVWGQGT | | CGTACCGGTGGTGGTTCAGGTGGCGGTGGC | |
| | | | | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCTACTCTTACTCTATC | |
| | | | | | CATTGGGTGCGCCAGGCACCTGGTAAAGGT | |
| | | | | | CTGGAATGGGTTGCCGAATTTTCTGATCAG | |
| | | | | | TCTGGTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTCTTACTACCAGTACGC | |
| | | | | | ATTTGATGTTTGGGGTCAGGGCACC | |
| FL_2 | GPYYPA | 179 | KPGKAPKLLIYSASFLYSGVPSRFSG | 180 | AAACCGGGTAAAGCACCGAAACTGCTGAT | 181 |
| | FDV | | SRSGTDFTLTISSLQPEDFATYYCQQ | | TTATAGCGCCTCTTTCCTGTATAGCGGTGT | |
| | | | MYSSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNISYYGMHWVRQA | | GCAGCCGGAAGATTTTGCAACCTATTATTG | |
| | | | PGKGLEWVAEVAPSSGYTYYADSV | | CCAGCAA ATGTACTCTTCTCCTATCACCTT | |
| | | | KGRFTISADTSKNTAYLQMNSLRAE | | TGGCCAGGGCACCAAAGTTGAAATTAAAC | |
| | | | DTAVYYCSRGPYYPAFDVWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTTACTACGGTATG | |
| | | | | | CATTGGGTGCGCCAGGCACCTGGTAAAGGT | |
| | | | | | CTGGAATGGGTTGCCGAAGTTGCACCATCT | |
| | | | | | TCTGGTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGGTCCATACTACCCAGC | |
| | | | | | ATTTGATGTTTGGGGTCAGGGCACC | |
| FL_3 | YSYYSSR | 182 | KPGKAPKLLIYSASFLYSGVPSRFSG | 183 | AAACCGGGTAAAGCACCGAAACTGCTGAT | 184 |
| | NYAMDV | | SRSGTDFTLTISSLQPEDFATYYCQQ | | TTATAGCGCCTCTTTCCTGTATAGCGGTGT | |
| | | | SRSSPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLFDQYMHWVRQA | | GCAGCCGGAAGATTTTGCAACCTATTATTG | |
| | | | PGKGLEWVAMLYPGSGQTYYADS | | CCAGCAA TCTAGATCTTCTCCTGTTACCTT | |
| | | | VKGRFTISADTSKNTAYLQMNSLRA | | TGGCCAGGGCACCAAAGTTGAAATTAAAC | |
| | | | EDTAVYYCSRYSYYSSRNYAMDV | | GTACCGGTGGTGGTTCAGGTGGCGGTGGCA | |
| | | | WGQGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTTTGATCAGTACATG | |
| | | | | | CATTGGGTGCGCCAGGCACCTGGTAAAGGT | |
| | | | | | CTGGAATGGGTTGCCATGCTGTACCCAGGT | |
| | | | | | TCTGGTCAGACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACTCTTACTACTCTTCT | |
| | | | | | AGAAACTACGCAATGGATGTTTGGGGTCA | |
| | | | | | GGGCACC | |
| FL_4 | ESGMSM | 185 | KPGKAPKLLIYSASFLYSGVPSRFSG | 186 | AAACCGGGTAAAGCACCGAAACTGCTGAT | 187 |
| | DY | | SRSGTDFTLTISSLQPEDFATYYCQQ | | TTATAGCGCCTCTTTCCTGTATAGCGGTGT | |
| | | | YSYPVTFGQGTKVEIKRTGGGSGGG | | GCCGAGCCGTTTTAGCGGTAGCCGTAGCGG | |
| | | | GSGGGASEVQLVESGGGLVQPGGS | | CACCGATTTTACCCTGACCATTAGCAGCCT | |
| | | | LRLSCAASGFNIAGSVMHWVRQAP | | GCAGCCGGAAGATTTTGCAACCTATTATTG | |
| | | | GKGLEWVAGLNPASGYTIYADSVK | | CCAGCAA TACTCTTACCCTGTTACCTTTGG | |
| | | | GRFTISADTSKNTAYLQMNSLRAED | | CCAGGGCACCAAAGTTGAAATTAAACGTA | |
| | | | TAVYYCSRESGMSMDYWGQGT | | CCGGTGGTGGTTCAGGTGGCGGTGGCAGC | |
| | | | | | GGAGGCGGAGCCAGCGAAGTGCAGCTGGT | |
| | | | | | TGAAAGCGGTGGCGGTCTGGTTCAGCCAG | |
| | | | | | GTGGTAGCCTGCGTCTGAGCTGTGCAGCAA | |
| | | | | | GCGGTTTTAACATCGCAGGTTCTGTTATGC | |
| | | | | | ATTGGGTGCGCCAGGCACCTGGTAAAGGTC | |
| | | | | | TGGAATGGGTTGCCGGTCTGAACCCAGCAT | |
| | | | | | CTGGTTACACCATCTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGAATCTGGTATGTCTATG | |
| | | | | | GATTACTGGGGTCAGGGCACC | |
| FL_5 | VVSSEYY | 188 | KPGKAPKLLIYSASFLYSGVPSRFSG | 189 | AAACCGGGTAAAGCACCGAAACTGCTGAT | 190 |
| | AMDY | | SRSGTDFTLTISSLQPEDFATYYCQQ | | TTATAGCGCCTCTTTCCTGTATAGCGGTGT | |
| | | | YKYQPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIRYGAMHWVRQA | | GCAGCCGGAAGATTTTGCAACCTATTATTG | |
| | | | PGKGLEWVADLYGWSGNTYYADS | | CCAGCAA TACAAATACCAGCCTATCACCT | |
| | | | VKGRFTISADTSKNTAYLQMNSLRA | | TTGGCCAGGGCACCAAAGTTGAAATTAAA | |
| | | | EDTAVYYCSRVVSSEYYAMDYWG | | CGTACCGGTGGTGGTTCAGGTGGCGGTGGC | |
| | | | QGT | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCAGATACGGTGCAAT | |
| | | | | | GCATTGGGTGCGCCAGGCACCTGGTAAAG | |
| | | | | | GTCTGGAATGGGTTGCCGATCTGTACGGTT | |
| | | | | | GGTCTGGTAACACCTACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCGTTGTTTCTTCTGAA | |
| | | | | | TACTACGCAATGGATTACTGGGGTCAGGGC | |
| | | | | | ACC | |
| FL_6 | YYYAFD | 191 | KPGKAPKLLIYSASFLYSGVPSRFSG | 192 | AAACCGGGTAAAGCACCGAAACTGCTGAT | 193 |
| | Y | | SRSGTDFTLTISSLQPEDFATYYCQQ | | TTATAGCGCCTCTTTCCTGTATAGCGGTGT | |
| | | | GSESPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNLSYSGMHWVRQA | | GCAGCCGGAAGATTTTGCAACCTATTATTG | |
| | | | PGKGLEWVAAIYPRSGYTYYADSV | | CCAGCAA GGTTCTGAATCTCCTATCACCTT | |
| | | | KGRFTISADTSKNTAYLQMNSLRAE | | TGGCCAGGGCACCAAAGTTGAAATTAAAC | |
| | | | DTAVYYCSRYYYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACCTGTCTTACTCTGGTATG | |
| | | | | | CATTGGGTGCGCCAGGCACCTGGTAAAGGT | |
| | | | | | CTGGAATGGGTTGCCGCAATCTACCCAAGA | |
| | | | | | TCTGGTTACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACTACTACGCATTTGA | |
| | | | | | TTACTGGGGTCAGGGCACC | |
| FL_7 | HDSYHF | 194 | KPGKAPKLLIYSASFLYSGVPSRFSG | 195 | AAACCGGGTAAAGCACCGAAACTGCTGAT | 196 |
| | WYYFDY | | SRSGTDFTLTISSLQPEDFATYYCQQ | | TTATAGCGCCTCTTTCCTGTATAGCGGTGT | |
| | | | YYSYPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIYGSGMHWVRQA | | GCAGCCGGAAGATTTTGCAACCTATTATTG | |
| | | | PGKGLEWVAVVNPLSGNTYYADSV | | CCAGCAA TACTACTCTTACCCTATCACCTT | |
| | | | KGRFTISADTSKNTAYLQMNSLRAE | | TGGCCAGGGCACCAAAGTTGAAATTAAAC | |
| | | | DTAVYYCSRHDSYHFWYYFDYWG | | GTACCGGTGGTGGTTCAGGTGGCGGTGGCA | |
| | | | QGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACGGTTCTGGTATG | |
| | | | | | CATTGGGTGCGCCAGGCACCTGGTAAAGGT | |
| | | | | | CTGGAATGGGTTGCCGTTGTTAACCCACTG | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCCATGATTCTTACCATTTT | |
| | | | | | TGGTACTACTTTGATTACTGGGGTCAGGGC | |
| | | | | | ACC | |
| FL_8 | AMRYYS | 197 | KPGKAPKLLIYSASFLYSGVPSRFSG | 198 | AAACCGGGTAAAGCACCGAAACTGCTGAT | 199 |
| | YYMDY | | SRSGTDFTLTISSLQPEDFATYYCQQ | | TTATAGCGCCTCTTTCCTGTATAGCGGTGT | |
| | | | VYTSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIYWSWMHWVRQ | | GCAGCCGGAAGATTTTGCAACCTATTATTG | |
| | | | APGKGLEWVAQIGPGGDYTNYADS | | CCAGCAA GTTTACACCTCTCCTATCACCTT | |
| | | | VKGRFTISADTSKNTAYLQMNSLRA | | TGGCCAGGGCACCAAAGTTGAAATTAAAC | |
| | | | EDTAVYYCSRAMRYYSYYMDYW | | GTACCGGTGGTGGTTCAGGTGGCGGTGGCA | |
| | | | GQGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACTGGTCTTGGATG | |
| | | | | | CATTGGGTGCGCCAGGCACCTGGTAAAGGT | |
| | | | | | CTGGAATGGGTTGCCCAGATCGGTCCAGGT | |
| | | | | | GGTGATTACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGCAATGAGATACTACTC | |
| | | | | | TTACTACATGGATTACTGGGGTCAGGGCAC | |
| | | | | | C | |
| FL_9 | AHFSSYV | 200 | KPGKAPKLLIYSASFLYSGVPSRFSG | 201 | AAACCGGGTAAAGCACCGAAACTGCTGAT | 202 |
| | DY | | SRSGTDFTLTISSLQPEDFATYYCQQ | | TTATAGCGCCTCTTTCCTGTATAGCGGTGT | |
| | | | YLSLPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNISDQDIHWVRQAP | | GCAGCCGGAAGATTTTGCAACCTATTATTG | |
| | | | GKGLEWVAMIYPLDSYTWYADSV | | CCAGCAA TACCTGTCTCTGCCTGTTACCTT | |
| | | | KGRFTISADTSKNTAYLQMNSLRAE | | TGGCCAGGGCACCAAAGTTGAAATTAAAC | |
| | | | DTAVYYCSRAHFSSYVDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTGATCAGGATATC | |
| | | | | | CATTGGGTGCGCCAGGCACCTGGTAAAGGT | |
| | | | | | CTGGAATGGGTTGCCATGATCTACCCACTG | |
| | | | | | GATTCTTACACCTGGTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGCACATTTTTCTTCTTAC | |
| | | | | | GTTGATTACTGGGGTCAGGGCACC | |
| FL_10 | EYGSAM | 203 | KPGKAPKLLIYSASFLYSGVPSRFSG | 204 | AAACCGGGTAAAGCACCGAAACTGCTGAT | 205 |
| | DY | | SRSGTDFTLTISSLQPEDFATYYCQQ | | TTATAGCGCCTCTTTCCTGTATAGCGGTGT | |
| | | | YRYSPVTFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNIWYWRMHWVRQ | | GCAGCCGGAAGATTTTGCAACCTATTATTG | |
| | | | APGKGLEWVAELSPRSSYTSYADSV | | CCAGCAA TACAGATACTCTCCTGTTACCTT | |
| | | | KGRFTISADTSKNTAYLQMNSLRAE | | TGGCCAGGGCACCAAAGTTGAAATTAAAC | |
| | | | DTAVYYCSREYGSAMDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTGGTACTGGAGAATG | |
| | | | | | CATTGGGTGCGCCAGGCACCTGGTAAAGGT | |
| | | | | | CTGGAATGGGTTGCCGAACTGTCTCCAAGA | |
| | | | | | TCTTCTTACACCTCTTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGAATACGGTTCTGCAAT | |
| | | | | | GGATTACTGGGGTCAGGGCACC | |
| FL_11 | YEAGISE | 206 | KPGKAPKLLIYSASFLYSGVPSRFSG | 207 | AAACCGGGTAAAGCACCGAAACTGCTGAT | 208 |
| | AMDV | | SRSGTDFTLTISSLQPEDFATYYCQQ | | TTATAGCGCCTCTTTCCTGTATAGCGGTGT | |
| | | | GYMDPVTFGQGTKVEIKRTGGGSG | | GCCGAGCCGTTTTAGCGGTAGCCGTAGCGG | |
| | | | GGGSGGGASEVQLVESGGGLVQPG | | CACCGATTTTACCCTGACCATTAGCAGCCT | |
| | | | GSLRLSCAASGFNISSYGMHWVRQ | | GCAGCCGGAAGATTTTGCAACCTATTATTG | |
| | | | APGKGLEWVALLYPQYSYTNYADS | | CCAGCAA GGTTACATGGATCCTGTTACCTT | |
| | | | VKGRFTISADTSKNTAYLQMNSLRA | | TGGCCAGGGCACCAAAGTTGAAATTAAAC | |
| | | | EDTAVYYCSRYEAGISEAMDVWG | | GTACCGGTGGTGGTTCAGGTGGCGGTGGCA | |
| | | | QGT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTCTTCTTACGGTATG | |
| | | | | | CATTGGGTGCGCCAGGCACCTGGTAAAGGT | |
| | | | | | CTGGAATGGGTTGCCCTGCTGTACCCACAG | |
| | | | | | TACTCTTACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTACGAAGCAGGTATCTC | |
| | | | | | TGAAGCAATGGATGTTTGGGGTCAGGGCA | |
| | | | | | CC | |
| FL_12 | SLFSYQY | 209 | KPGKAPKLLIYSASFLYSGVPSRFSG | 210 | AAACCGGGTAAAGCACCGAAACTGCTGAT | 211 |
| | AFDY | | SRSGTDFTLTISSLQPEDFATYYCQQ | | TTATAGCGCCTCTTTCCTGTATAGCGGTGT | |
| | | | YFSYPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNVFQYTMHWVRQA | | GCAGCCGGAAGATTTTGCAACCTATTATTG | |
| | | | PGKGLEWVATIYPISGNTYYADSVK | | CCAGCAA TACTTTTCTTACCCTATCACCTT | |
| | | | GRFTISADTSKNTAYLQMNSLRAED | | TGGCCAGGGCACCAAAGTTGAAATTAAAC | |
| | | | TAVYYCSRSLFSYQYAFDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACGTTTTTCAGTACACCATG | |
| | | | | | CATTGGGTGCGCCAGGCACCTGGTAAAGGT | |
| | | | | | CTGGAATGGGTTGCCACCATCTACCCAATC | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTCTCTGTTTTCTTACCAG | |
| | | | | | TACGCATTTGATTACTGGGGTCAGGGCACC | |
| FL_13 | SSRSMA | 212 | KPGKAPKLLIYSASFLYSGVPSRFSG | 213 | AAACCGGGTAAAGCACCGAAACTGCTGAT | 214 |
| | LDY | | SRSGTDFTLTISSLQPEDFATYYCQQ | | TTATAGCGCCTCTTTCCTGTATAGCGGTGT | |
| | | | YHSYPMTFGQGTKVEIKRTGGGSG | | GCCGAGCCGTTTTAGCGGTAGCCGTAGCGG | |
| | | | GGGSGGGASEVQLVESGGGLVQPG | | CACCGATTTTACCCTGACCATTAGCAGCCT | |
| | | | GSLRLSCAASGFNIYNSGMHWVRQ | | GCAGCCGGAAGATTTTGCAACCTATTATTG | |
| | | | APGKGLEWVAVVWGGSGNTYYAD | | CCAGCAA TACCATTCTTACCCTATGACCTT | |
| | | | SVKGRFTISADTSKNTAYLQMNSLR | | TGGCCAGGGCACCAAAGTTGAAATTAAAC | |
| | | | AEDTAVYYCSRSSRSMALDYWGQ | | GTACCGGTGGTGGTTCAGGTGGCGGTGGCA | |
| | | | GT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACAACTCTGGTATG | |
| | | | | | CATTGGGTGCGCCAGGCACCTGGTAAAGGT | |
| | | | | | CTGGAATGGGTTGCCGTTGTTTGGGGTGGT | |
| | | | | | TCTGGTAACACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCTCTTCTAGATCTATGGC | |
| | | | | | ACTGGATTACTGGGGTCAGGGCACC | |
| BETA_ | WYGPY | 215 | KPGKAPKLLIYSASFLYSGVPSRFSG | 216 | AAACCGGGTAAAGCACCGAAACTGCTGAT | 217 |
| 1 | MDY | | SRSGTDFTLTISSLQPEDFATYYCQQ | | TTATAGCGCCTCTTTTCTGTATAGCGGTGTG | |
| | | | GRYDPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNLSYYIMHWVRQ | | CAGCCGGAAGATTTTGCAACCTATTATTGC | |
| | | | APGKGLEWVAYVYPGYDSTYYAD | | CAGCAA GGTAGATACGATCCTGTTACCTTT | |
| | | | SVKGRFTISADTSKNTAYLQMNSLR | | GGCCAGGGCACCAAAGTTGAAATTAAACG | |
| | | | AEDTAVYYCSRWYGPYMDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACCTGTCTTACTACATCATGC | |
| | | | | | ATTGGGTGCGCCAGGCACCTGGTAAAGGTC | |
| | | | | | TGGAATGGGTTGCCTACGTTTACCCAGGTT | |
| | | | | | ACGATTCTACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTGGTACGGTCCATACATG | |
| | | | | | GATTACTGGGGTCAGGGCACC | |
| BETA_ | SSRSLAL | 218 | KPGKAPKLLIYSASFLYSGVPSRFSG | 219 | AAACCGGGTAAAGCACCGAAACTGCTGAT | 220 |
| 2 | DY | | SRSGTDFTLTISSLQPEDFATYYCQQ | | TTATAGCGCCTCTTTTCTGTATAGCGGTGTG | |
| | | | YHSYPMTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIYNSGMHWVRQ | | CAGCCGGAAGATTTTGCAACCTATTATTGC | |
| | | | APGKGLEWVAVVWGGSGNTYYAD | | CAGCAA TACCATTCTTACCCTATGACCTTT | |
| | | | SVKGRFTISADTSKNTAYLQMNSLR | | GGCCAGGGCACCAAAGTTGAAATTAAACG | |
| | | | AEDTAVYYCSRSSRSLALDYWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACAACTCTGGTATGC | |
| | | | | | ATTGGGTGCGCCAGGCACCTGGTAAAGGTC | |
| | | | | | TGGAATGGGTTGCCGTTGTTTGGGGTGGTT | |
| | | | | | CTGGTAACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTTCTAGATCTCTGGCA | |
| | | | | | CTGGATTACTGGGGTCAGGGCACC | |
| BETA_ | MDSASSS | 221 | KPGKAPKLLIYSASFLYSGVPSRFSG | 222 | AAACCGGGTAAAGCACCGAAACTGCTGAT | 223 |
| 3 | KTSYHY | | SRSGTDFTLTISSLQPEDFATYYCQQ | | TTATAGCGCCTCTTTTCTGTATAGCGGTGTG | |
| | GSQYSF | | STYPPVTFGQGTKVEIKRTGGGSGG | | CCGAGCCGTTTTAGCGGTAGCCGTAGCGGC | |
| | DY | | GGSGGGASEVQLVESGGGLVQPGG | | ACCGATTTTACCCTGACCATTAGCAGCCTG | |
| | | | SLRLSCAASGFNISESIMHWVRQAP | | CAGCCGGAAGATTTTGCAACCTATTATTGC | |
| | | | GKGLEWVAHVYPGYGYTWYADSV | | CAGCAA TCTACCTACCCACCTGTTACCTTT | |
| | | | KGRFTISADTSKNTAYLQMNSLRAE | | GGCCAGGGCACCAAAGTTGAAATTAAACG | |
| | | | DTAVYYCSRMDSASSSKTSYHYGS | | TACCGGTGGTGGTTCAGGTGGCGGTGGCAG | |
| | | | QYSFDYWGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTGAATCTATCATGC | |
| | | | | | ATTGGGTGCGCCAGGCACCTGGTAAAGGTC | |
| | | | | | TGGAATGGGTTGCCCATGTTTACCCAGGTT | |
| | | | | | ACGGTTACACCTGGTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCATGGATTCTGCATCTTCTT | |
| | | | | | CTAAAACCTCTTACCATTACGGTTCTCAGT | |
| | | | | | ACTCTTTTGATTACTGGGGTCAGGGCACC | |
| BETA_ | GMVSSY | 224 | KPGKAPKLLIYSASFLYSGVPSRFSG | 225 | AAACCGGGTAAAGCACCGAAACTGCTGAT | 226 |
| 4 | MDY | | SRSGTDFTLTISSLQPEDFATYYCQQ | | TTATAGCGCCTCTTTTCTGTATAGCGGTGTG | |
| | | | IRYNPMTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIYYSEMHWVRQA | | CAGCCGGAAGATTTTGCAACCTATTATTGC | |
| | | | PGKGLEWVANISPGGGSTYYADSV | | CAGCAA ATCAGATACAACCCTATGACCTT | |
| | | | KGRFTISADTSKNTAYLQMNSLRAE | | TGGCCAGGGCACCAAAGTTGAAATTAAAC | |
| | | | DTAVYYCSRGMVSSYMDYWGQGT | | GTACCGGTGGTGGTTCAGGTGGCGGTGGCA | |
| | | | | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACATCTACTACTCTGAAATG | |
| | | | | | CATTGGGTGCGCCAGGCACCTGGTAAAGGT | |
| | | | | | CTGGAATGGGTTGCCAACATCTCTCCAGGT | |
| | | | | | GGTGGTTCTACCTACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGGTATGGTTTCTTCTTAC | |
| | | | | | ATGGATTACTGGGGTCAGGGCACC | |
| BETA_ | SIYSAFD | 227 | KPGKAPKLLIYSASFLYSGVPSRFSG | 228 | AAACCGGGTAAAGCACCGAAACTGCTGAT | 229 |
| 5 | Y | | SRSGTDFTLTISSLQPEDFATYYCQQ | | TTATAGCGCCTCTTTTCTGTATAGCGGTGTG | |
| | | | DSSSTPFTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNITGYHMHWVRQ | | CAGCCGGAAGATTTTGCAACCTATTATTGC | |
| | | | APGKGLEWVAYISGGGYYTYYADS | | CAGCAA GATTCTTCTTCTACCCCTTTTACC | |
| | | | VKGRFTISADTSKNTAYLQMNSLRA | | TTTGGCCAGGGCACCAAAGTTGAAATTAAA | |
| | | | EDTAVYYCSRSIYSAFDYWGQGT | | CGTACCGGTGGTGGTTCAGGTGGCGGTGGC | |
| | | | | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCACCGGTTACCATAT | |
| | | | | | GCATTGGGTGCGCCAGGCACCTGGTAAAG | |
| | | | | | GTCTGGAATGGGTTGCCTACATCTCTGGTG | |
| | | | | | GTGGTTACTACACCTACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCTCTATCTACTCTGCA | |
| | | | | | TTTGATTACTGGGGTCAGGGCACC | |
| BETA_ | ASGYAFS | 230 | KPGKAPKLLIYSASFLYSGVPSRFSG | 231 | AAACCGGGTAAAGCACCGAAACTGCTGAT | 232 |
| 6 | ALDY | | SRSGTDFTLTISSLQPEDFATYYCQQ | | TTATAGCGCCTCTTTTCTGTATAGCGGTGTG | |
| | | | YNYVPYTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVYSDSMHWVRQ | | CAGCCGGAAGATTTTGCAACCTATTATTGC | |
| | | | APGKGLEWVAVIGPDSGYTYYADS | | CAGCAA TACAACTACGTTCCTTACACCTTT | |
| | | | VKGRFTISADTSKNTAYLQMNSLRA | | GGCCAGGGCACCAAAGTTGAAATTAAACG | |
| | | | EDTAVYYCSRASGYAFSALDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACTCTGATTCTATGCA | |
| | | | | | TTGGGTGCGCCAGGCACCTGGTAAAGGTCT | |
| | | | | | GGAATGGGTTGCCGTTATCGGTCCAGATTC | |
| | | | | | TGGTTACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGCATCTGGTTACGCATTTT | |
| | | | | | CTGCACTGGATTACTGGGGTCAGGGCACC | |
| BETA_ | GYYSGY | 233 | KPGKAPKLLIYSASFLYSGVPSRFSG | 234 | AAACCGGGTAAAGCACCGAAACTGCTGAT | 235 |
| 7 | SFDY | | SRSGTDFTLTISSLQPEDFATYYCQQ | | TTATAGCGCCTCTTTTCTGTATAGCGGTGTG | |
| | | | YMYEPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIITYDMHWVRQA | | CAGCCGGAAGATTTTGCAACCTATTATTGC | |
| | | | PGKGLEWVAVIVDDLGYTNYADSV | | CAGCAA TACATGTACGAACCTGTTACCTTT | |
| | | | KGRFTISADTSKNTAYLQMNSLRAE | | GGCCAGGGCACCAAAGTTGAAATTAAACG | |
| | | | DTAVYYCSRGYYSGYSFDYWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCATCACCTACGATATGC | |
| | | | | | ATTGGGTGCGCCAGGCACCTGGTAAAGGTC | |
| | | | | | TGGAATGGGTTGCCGTTATCGTTGATGATC | |
| | | | | | TGGGTTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTTACTACTCTGGTTAC | |
| | | | | | TCTTTTGATTACTGGGGTCAGGGCACC | |
| BETA_ | AYYYYA | 236 | KPGKAPKLLIYSASFLYSGVPSRFSG | 237 | AAACCGGGTAAAGCACCGAAACTGCTGAT | 238 |
| 8 | FAY | | SRSGTDFTLTISSLQPEDFATYYCQQ | | TTATAGCGCCTCTTTTCTGTATAGCGGTGTG | |
| | | | VWLSPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIYDTGIHWVRQAP | | CAGCCGGAAGATTTTGCAACCTATTATTGC | |
| | | | GKGLEWVATIYDFSSNTYYADSVK | | CAGCAA GTTTGGCTGTCTCCTATCACCTTT | |
| | | | GRFTISADTSKNTAYLQMNSLRAED | | GGCCAGGGCACCAAAGTTGAAATTAAACG | |
| | | | TAVYYCSRAYYYYAFAYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACGATACCGGTATCC | |
| | | | | | ATTGGGTGCGCCAGGCACCTGGTAAAGGTC | |
| | | | | | TGGAATGGGTTGCCACCATCTACGATTTTT | |
| | | | | | CTTCTAACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCATACTACTACTACGCA | |
| | | | | | TTTGCATACTGGGGTCAGGGCACC | |
| BETA_ | LHIHYM | 239 | KPGKAPKLLIYSASFLYSGVPSRFSG | 240 | AAACCGGGTAAAGCACCGAAACTGCTGAT | 241 |
| 9 | ALDV | | SRSGTDFTLTISSLQPEDFATYYCQQ | | TTATAGCGCCTCTTTTCTGTATAGCGGTGTG | |
| | | | SLSKPFITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISGIGMHWVRQAP | | CAGCCGGAAGATTTTGCAACCTATTATTGC | |
| | | | GKGLEWVAWVSPKSGYTYYADSV | | CAGCAA TCTCTGTCTAAACCTTTTATCACC | |
| | | | KGRFTISADTSKNTAYLQMNSLRAE | | TTTGGCCAGGGCACCAAAGTTGAAATTAAA | |
| | | | DTAVYYCSRLHIHYMALDVWGQG | | CGTACCGGTGGTGGTTCAGGTGGCGGTGGC | |
| | | | T | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCTCTGGTATCGGTAT | |
| | | | | | GCATTGGGTGCGCCAGGCACCTGGTAAAG | |
| | | | | | GTCTGGAATGGGTTGCCTGGGTTTCTCCAA | |
| | | | | | AATCTGGTTACACCTACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCCTGCATATCCATTA | |
| | | | | | CATGGCACTGGATGTTTGGGGTCAGGGCAC | |
| | | | | | C | |
| BETA_ | SYYSYFD | 242 | KPGKAPKLLIYSASFLYSGVPSRFSG | 243 | AAACCGGGTAAAGCACCGAAACTGCTGAT | 244 |
| 10 | Y | | SRSGTDFTLTISSLQPEDFATYYCQQ | | TTATAGCGCCTCTTTTCTGTATAGCGGTGTG | |
| | | | SYWLPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVFPGAMHWVRQA | | CAGCCGGAAGATTTTGCAACCTATTATTGC | |
| | | | PGKGLEWVAMVYDDSSNTYYADS | | CAGCAA TCTTACTGGCTGCCTATCACCTTT | |
| | | | VKGRFTISADTSKNTAYLQMNSLRA | | GGCCAGGGCACCAAAGTTGAAATTAAACG | |
| | | | EDTAVYYCSRSYYSYFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTTTCCAGGTGCAATGC | |
| | | | | | ATTGGGTGCGCCAGGCACCTGGTAAAGGTC | |
| | | | | | TGGAATGGGTTGCCATGGTTTACGATGATT | |
| | | | | | CTTCTAACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTTACTACTCTTACTTTG | |
| | | | | | ATTACTGGGGTCAGGGCACC | |
| BETA_ | HTPYYM | 245 | KPGKAPKLLIYSASFLYSGVPSRFSG | 246 | AAACCGGGTAAAGCACCGAAACTGCTGAT | 247 |
| 11 | DY | | SRSGTDFTLTISSLQPEDFATYYCQQ | | TTATAGCGCCTCTTTTCTGTATAGCGGTGTG | |
| | | | HYNTPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSRQGIHWVRQAP | | CAGCCGGAAGATTTTGCAACCTATTATTGC | |
| | | | GKGLEWVAHIWGDSGYTYYADSV | | CAGCAA CATTACAACACCCCTATCACCTTT | |
| | | | KGRFTISADTSKNTAYLQMNSLRAE | | GGCCAGGGCACCAAAGTTGAAATTAAACG | |
| | | | DTAVYYCSRHTPYYMDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTAGACAGGGTATCC | |
| | | | | | ATTGGGTGCGCCAGGCACCTGGTAAAGGTC | |
| | | | | | TGGAATGGGTTGCCCATATCTGGGGTGATT | |
| | | | | | CTGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCCATACCCCATACTACATG | |
| | | | | | GATTACTGGGGTCAGGGCACC | |
| BETA_ | SPYSSFD | 248 | KPGKAPKLLIYSASFLYSGVPSRFSG | 249 | AAACCGGGTAAAGCACCGAAACTGCTGAT | 250 |
| 12 | Y | | SRSGTDFTLTISSLQPEDFATYYCQQ | | TTATAGCGCCTCTTTTCTGTATAGCGGTGTG | |
| | | | YVSWPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIEFSGMHWVRQAP | | CAGCCGGAAGATTTTGCAACCTATTATTGC | |
| | | | GKGLEWVAFIYSDSGYTNYADSVK | | CAGCAA TACGTTTCTTGGCCTATCACCTTT | |
| | | | GRFTISADTSKNTAYLQMNSLRAED | | GGCCAGGGCACCAAAGTTGAAATTAAACG | |
| | | | TAVYYCSRSPYSSFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCGAATTTTCTGGTATGC | |
| | | | | | ATTGGGTGCGCCAGGCACCTGGTAAAGGTC | |
| | | | | | TGGAATGGGTTGCCTTTATCTACTCTGATTC | |
| | | | | | TGGTTACACCAACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCTCTCCATACTCTTCTTTTGA | |
| | | | | | TTACTGGGGTCAGGGCACC | |
| BETA_ | SYSYSYF | 251 | KPGKAPKLLIYSASFLYSGVPSRFSG | 252 | AAACCGGGTAAAGCACCGAAACTGCTGAT | 253 |
| 13 | DY | | SRSGTDFTLTISSLQPEDFATYYCQQ | | TTATAGCGCCTCTTTTCTGTATAGCGGTGTG | |
| | | | AYYDPFTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVYYSGMHWVRQA | | CAGCCGGAAGATTTTGCAACCTATTATTGC | |
| | | | PGKGLEWVAVISGSGYTYYADSVK | | CAGCAA GCATACTACGATCCTTTTACCTTT | |
| | | | GRFTISADTSKNTAYLQMNSLRAED | | GGCCAGGGCACCAAAGTTGAAATTAAACG | |
| | | | TAVYYCSRSYSYSYFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACTACTCTGGTATGC | |
| | | | | | ATTGGGTGCGCCAGGCACCTGGTAAAGGTC | |
| | | | | | TGGAATGGGTTGCCGTTATCTCTGGTTCTG | |
| | | | | | GTTACACCTACTATGCCGATAGCGTGAAAG | |
| | | | | | GTCGCTTTACCATTAGCGCAGATACCAGCA | |
| | | | | | AAAATACCGCATACCTGCAGATGAATAGTC | |
| | | | | | TGCGTGCAGAGGATACAGCAGTGTATTATT | |
| | | | | | GTAGCCGCTCTTACTCTTACTCTTACTTTGA | |
| | | | | | TTACTGGGGTCAGGGCACC | |
| BETA_ | EYMSQT | 254 | KPGKAPKLLIYSASFLYSGVPSRFSG | 255 | AAACCGGGTAAAGCACCGAAACTGCTGAT | 256 |
| 14 | FDY | | SRSGTDFTLTISSLQPEDFATYYCQQ | | TTATAGCGCCTCTTTTCTGTATAGCGGTGTG | |
| | | | NRYVPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVSGSGIHWVRQA | | CAGCCGGAAGATTTTGCAACCTATTATTGC | |
| | | | PGKGLEWVAQVSPWSYNTYYADS | | CAGCAA AACAGATACGTTCCTGTTACCTTT | |
| | | | VKGRFTISADTSKNTAYLQMNSLRA | | GGCCAGGGCACCAAAGTTGAAATTAAACG | |
| | | | EDTAVYYCSREYMSQTFDYWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTGGTTCTGGTATCCA | |
| | | | | | TTGGGTGCGCCAGGCACCTGGTAAAGGTCT | |
| | | | | | GGAATGGGTTGCCCAGGTTTCTCCATGGTC | |
| | | | | | TTACAACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGAATACATGTCTCAGACCT | |
| | | | | | TTGATTACTGGGGTCAGGGCACC | |
| BETA_ | VWYPYA | 257 | KPGKAPKLLIYSASFLYSGVPSRFSG | 258 | AAACCGGGTAAAGCACCGAAACTGCTGAT | 259 |
| 15 | FDY | | SRSGTDFTLTISSLQPEDFATYYCQQ | | TTATAGCGCCTCTTTTCTGTATAGCGGTGTG | |
| | | | YSSSPITFGQGTKVEIKRTGGGSGGG | | CCGAGCCGTTTTAGCGGTAGCCGTAGCGGC | |
| | | | GSGGGASEVQLVESGGGLVQPGGS | | ACCGATTTTACCCTGACCATTAGCAGCCTG | |
| | | | LRLSCAASGFNISEYDIHWVRQAPG | | CAGCCGGAAGATTTTGCAACCTATTATTGC | |
| | | | KGLEWVADVDDQMGYTYYADSVK | | CAGCAA TACTCTTCTTCTCCTATCACCTTT | |
| | | | GRFTISADTSKNTAYLQMNSLRAED | | GGCCAGGGCACCAAAGTTGAAATTAAACG | |
| | | | TAVYYCSRVWYPYAFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTGAATACGATATCC | |
| | | | | | ATTGGGTGCGCCAGGCACCTGGTAAAGGTC | |
| | | | | | TGGAATGGGTTGCCGATGTTGATGATCAGA | |
| | | | | | TGGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGTTTGGTACCCATACGCA | |
| | | | | | TTTGATTACTGGGGTCAGGGCACC | |
| BETA_ | HDYYYL | 260 | KPGKAPKLLIYSASFLYSGVPSRFSG | 261 | AAACCGGGTAAAGCACCGAAACTGCTGAT | 262 |
| 16 | DY | | SRSGTDFTLTISSLQPEDFATYYCQQ | | TTATAGCGCCTCTTTTCTGTATAGCGGTGTG | |
| | | | LYSTPVTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIGYYGIHWVRQAP | | CAGCCGGAAGATTTTGCAACCTATTATTGC | |
| | | | GKGLEWVAHISPGPGNTYYADSVK | | CAGCAA CTGTACTCTACCCCTGTTACCTTT | |
| | | | GRFTISADTSKNTAYLQMNSLRAED | | GGCCAGGGCACCAAAGTTGAAATTAAACG | |
| | | | TAVYYCSRHDYYYLDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCGGTTACTACGGTATCC | |
| | | | | | ATTGGGTGCGCCAGGCACCTGGTAAAGGTC | |
| | | | | | TGGAATGGGTTGCCCATATCTCTCCAGGTC | |
| | | | | | CAGGTAACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCCATGATTACTACTACCTG | |
| | | | | | GATTACTGGGGTCAGGGCACC | |
| BETA_ | GNPYGA | 263 | KPGKAPKLLIYSASFLYSGVPSRFSG | 264 | AAACCGGGTAAAGCACCGAAACTGCTGAT | 265 |
| 17 | FDY | | SRSGTDFTLTISSLQPEDFATYYCQQ | | TTATAGCGCCTCTTTTCTGTATAGCGGTGTG | |
| | | | SRYYPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIAYYSMHWVRQA | | CAGCCGGAAGATTTTGCAACCTATTATTGC | |
| | | | PGKGLEWVAAVYPYSDYTYYADS | | CAGCAA TCTAGATACTACCCTATCACCTTT | |
| | | | VKGRFTISADTSKNTAYLQMNSLRA | | GGCCAGGGCACCAAAGTTGAAATTAAACG | |
| | | | EDTAVYYCSRGNPYGAFDYWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCGCATACTACTCTATGC | |
| | | | | | ATTGGGTGCGCCAGGCACCTGGTAAAGGTC | |
| | | | | | TGGAATGGGTTGCCGCAGTTTACCCATACT | |
| | | | | | CTGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTAACCCATACGGTGCA | |
| | | | | | TTTGATTACTGGGGTCAGGGCACC | |
| BETA_ | AHDYSY | 266 | KPGKAPKLLIYSASFLYSGVPSRFSG | 267 | AAACCGGGTAAAGCACCGAAACTGCTGAT | 268 |
| 18 | RAFDH | | SRSGTDFTLTISSLQPEDFATYYCQQ | | TTATAGCGCCTCTTTTCTGTATAGCGGTGTG | |
| | | | IREFPHTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNITYYDMHWVRQA | | CAGCCGGAAGATTTTGCAACCTATTATTGC | |
| | | | PGKGLEWVAAIIDDSGDTYYADSV | | CAGCAA ATCAGAGAATTTCCTCATACCTTT | |
| | | | KGRFTISADTSKNTAYLQMNSLRAE | | GGCCAGGGCACCAAAGTTGAAATTAAACG | |
| | | | DTAVYYCSRAHDYSYRAFDHWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCACCTACTACGATATGC | |
| | | | | | ATTGGGTGCGCCAGGCACCTGGTAAAGGTC | |
| | | | | | TGGAATGGGTTGCCGCAATCATCGATGATT | |
| | | | | | CTGGTGATACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCACATGATTACTCTTAC | |
| | | | | | AGAGCATTTGATCACTGGGGTCAGGGCACC | |
| BETA_ | YEPGYA | 269 | KPGKAPKLLIYSASFLYSGVPSRFSG | 270 | AAACCGGGTAAAGCACCGAAACTGCTGAT | 271 |
| 19 | LDY | | SRSGTDFTLTISSLQPEDFATYYCQQ | | TTATAGCGCCTCTTTTCTGTATAGCGGTGTG | |
| | | | SYYYPFTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIRHYDMHWVRQA | | CAGCCGGAAGATTTTGCAACCTATTATTGC | |
| | | | PGKGLEWVAHILPYPGYTYYADSV | | CAGCAA TCTTACTACTACCCTTTTACCTTT | |
| | | | KGRFTISADTSKNTAYLQMNSLRAE | | GGCCAGGGCACCAAAGTTGAAATTAAACG | |
| | | | DTAVYYCSRYEPGYALDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCAGACATTACGATATGC | |
| | | | | | ATTGGGTGCGCCAGGCACCTGGTAAAGGTC | |
| | | | | | TGGAATGGGTTGCCCATATCCTGCCATACC | |
| | | | | | CTGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTACGAACCAGGTTACGCA | |
| | | | | | CTGGATTACTGGGGTCAGGGCACC | |
| BETA_ | YSYDAID | 272 | KPGKAPKLLIYSASFLYSGVPSRFSG | 273 | AAACCGGGTAAAGCACCGAAACTGCTGAT | 274 |
| 20 | Y | | SRSGTDFTLTISSLQPEDFATYYCQQ | | TTATAGCGCCTCTTTTCTGTATAGCGGTGTG | |
| | | | ISVTPITFGQGTKVEIKRTGGGSGGG | | CCGAGCCGTTTTAGCGGTAGCCGTAGCGGC | |
| | | | GSGGGASEVQLVESGGGLVQPGGS | | ACCGATTTTACCCTGACCATTAGCAGCCTG | |
| | | | LRLSCAASGFNISHQSMHWVRQAP | | CAGCCGGAAGATTTTGCAACCTATTATTGC | |
| | | | GKGLEWVAYVYPDSDYTEYADSV | | CAGCAA ATCTCTGTTACCCCTATCACCTTT | |
| | | | KGRFTISADTSKNTAYLQMNSLRAE | | GGCCAGGGCACCAAAGTTGAAATTAAACG | |
| | | | DTAVYYCSRYSYDAIDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTCATCAGTCTATGC | |
| | | | | | ATTGGGTGCGCCAGGCACCTGGTAAAGGTC | |
| | | | | | TGGAATGGGTTGCCTACGTTTACCCAGATT | |
| | | | | | CTGATTACACCGAATATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTACTCTTACGATGCAATT | |
| | | | | | GATTACTGGGGTCAGGGCACC | |
| GAMMA_ | ATSYGY | 275 | KPGKAPKLLIYSASFLYSGVPSRFSG | 276 | AAACCGGGTAAAGCACCGAAACTGCTGAT | 27 |
| 1 | SAMDY | | SRSGTDFTLTISSLQPEDFATYYCQQ | | TTATAGCGCCTCTTTTCTGTATAGCGGTGTG | |
| | | | YYSSPWTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNISSPVMHWVRQA | | CAGCCGGAAGATTTTGCAACCTATTATTGC | |
| | | | PGKGLEWVATLDPDYSYTNYADSV | | CAGCAA TACTACTCTTCTCCTTGGACCTTT | |
| | | | KGRFTISADTSKNTAYLQMNSLRAE | | GGCCAGGGCACCAAAGTTGAAATTAAACG | |
| | | | DTAVYYCSRATSYGYSAMDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTCTCCAGTTATGCA | |
| | | | | | TTGGGTGCGCCAGGCACCTGGTAAAGGTCT | |
| | | | | | GGAATGGGTTGCCACCCTGGATCCAGATTA | |
| | | | | | CTCTTACACCAACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGCAACCTCTTACGGTTACT | |
| | | | | | CTGCAATGGATTACTGGGGTCAGGGCACC | |
| GAMMA_ | SIYWDA | 278 | KPGKAPKLLIYSASFLYSGVPSRFSG | 279 | AAACCGGGTAAAGCACCGAAACTGCTGAT | 280 |
| 2 | AMDY | | SRSGTDFTLTISSLQPEDFATYYCQQ | | TTATAGCGCCTCTTTTCTGTATAGCGGTGTG | |
| | | | SSNYPHTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSSTYMHWVRQA | | CAGCCGGAAGATTTTGCAACCTATTATTGC | |
| | | | PGKGLEWVAMFYYYRGYTYYADS | | CAGCAA TCTTCTAACTACCCTCATACCTTT | |
| | | | VKGRFTISADTSKNTAYLQMNSLRA | | GGCCAGGGCACCAAAGTTGAAATTAAACG | |
| | | | EDTAVYYCSRSIYWDAAMDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTTCTACCTACATGCA | |
| | | | | | TTGGGTGCGCCAGGCACCTGGTAAAGGTCT | |
| | | | | | GGAATGGGTTGCCATGTTTTACTACTACAG | |
| | | | | | AGGTTACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCTCTATCTACTGGGATGCAG | |
| | | | | | CAATGGATTACTGGGGTCAGGGCACC | |
| GAMMA_ | GSYGHY | 281 | KPGKAPKLLIYSASFLYSGVPSRFSG | 282 | AAACCGGGTAAAGCACCGAAACTGCTGAT | 283 |
| 3 | GYSMDY | | SRSGTDFTLTISSLQPEDFATYYCQQ | | TTATAGCGCCTCTTTTCTGTATAGCGGTGTG | |
| | | | YSIEPDTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISSSIIHWVRQAPG | | CAGCCGGAAGATTTTGCAACCTATTATTGC | |
| | | | KGLEWVAMFPPYSGNTYYADSVK | | CAGCAA TACTCTATCGAACCTGATACCTTT | |
| | | | GRFTISADTSKNTAYLQMNSLRAED | | GGCCAGGGCACCAAAGTTGAAATTAAACG | |
| | | | TAVYYCSRGSYGHYGYSMDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTCTTCTATCATCCA | |
| | | | | | TTGGGTGCGCCAGGCACCTGGTAAAGGTCT | |
| | | | | | GGAATGGGTTGCCATGTTTCCACCATACTC | |
| | | | | | TGGTAACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGGTTCTTACGGTCATTACG | |
| | | | | | GTTACTCTATGGATTACTGGGGTCAGGGCA | |
| | | | | | CC | |
| GAMMA_ | SGSYAFS | 284 | KPGKAPKLLIYSASFLYSGVPSRFSG | 285 | AAACCGGGTAAAGCACCGAAACTGCTGAT | 286 |
| 4 | AMDV | | SRSGTDFTLTISSLQPEDFATYYCQQ | | TTATAGCGCCTCTTTTCTGTATAGCGGTGTG | |
| | | | YSYMPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVAGSDIHWVRQ | | CAGCCGGAAGATTTTGCAACCTATTATTGC | |
| | | | APGKGLEWVAFVYPGYGVTNYAD | | CAGCAA TACTCTTACATGCCTGTTACCTTT | |
| | | | SVKGRFTISADTSKNTAYLQMNSLR | | GGCCAGGGCACCAAAGTTGAAATTAAACG | |
| | | | AEDTAVYYCSRSGSYAFSAMDVW | | TACCGGTGGTGGTTCAGGTGGCGGTGGCAG | |
| | | | GQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTGCAGGTTCTGATATCC | |
| | | | | | ATTGGGTGCGCCAGGCACCTGGTAAAGGTC | |
| | | | | | TGGAATGGGTTGCCTTTGTTTACCCAGGTT | |
| | | | | | ACGGTGTTACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTGGTTCTTACGCATTTT | |
| | | | | | CTGCAATGGATGTTTGGGGTCAGGGCACC | |
| GAMMA_ | ADEYWG | 287 | KPGKAPKLLIYSASFLYSGVPSRFSG | 28 | AAACCGGGTAAAGCACCGAAACTGCTGAT | 289 |
| 5 | AVDY | | SRSGTDFTLTISSLQPEDFATYYCQQ | | TTATAGCGCCTCTTTTCTGTATAGCGGTGTG | |
| | | | TLIYPITFGQGTKVEIKRTGGGSGGG | | CCGAGCCGTTTTAGCGGTAGCCGTAGCGGC | |
| | | | GSGGGASEVQLVESGGGLVQPGGS | | ACCGATTTTACCCTGACCATTAGCAGCCTG | |
| | | | LRLSCAASGFNISSYMIHWVRQAPG | | CAGCCGGAAGATTTTGCAACCTATTATTGC | |
| | | | KGLEWVAVIYGSSDYTYYADSVKG | | CAGCAA ACCCTGATCTACCCTATCACCTTT | |
| | | | RFTISADTSKNTAYLQMNSLRAEDT | | GGCCAGGGCACCAAAGTTGAAATTAAACG | |
| | | | AVYYCSRADEYWGAVDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTCTTACATGATCCA | |
| | | | | | TTGGGTGCGCCAGGCACCTGGTAAAGGTCT | |
| | | | | | GGAATGGGTTGCCGTTATCTACGGTTCTTC | |
| | | | | | TGATTACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGCAGATGAATACTGGGGTG | |
| | | | | | CAGTTGATTACTGGGGTCAGGGCACC | |
| GAMMA_ | GQTESA | 290 | KPGKAPKLLIYSASFLYSGVPSRFSG | 291 | AAACCGGGTAAAGCACCGAAACTGCTGAT | 292 |
| 6 | VDV | | SRSGTDFTLTISSLQPEDFATYYCQQ | | TTATAGCGCCTCTTTTCTGTATAGCGGTGTG | |
| | | | RYTIPITFGQGTKVEIKRTGGGSGGG | | CCGAGCCGTTTTAGCGGTAGCCGTAGCGGC | |
| | | | GSGGGASEVQLVESGGGLVQPGGS | | ACCGATTTTACCCTGACCATTAGCAGCCTG | |
| | | | LRLSCAASGFNLGYYMMHWVRQA | | CAGCCGGAAGATTTTGCAACCTATTATTGC | |
| | | | PGKGLEWVAMFYGDSGYTNYADS | | CAGCAA AGATACACCATCCCTATCACCTTT | |
| | | | VKGRFTISADTSKNTAYLQMNSLRA | | GGCCAGGGCACCAAAGTTGAAATTAAACG | |
| | | | EDTAVYYCSRGQTESAVDVWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACCTGGGTTACTACATGATGC | |
| | | | | | ATTGGGTGCGCCAGGCACCTGGTAAAGGTC | |
| | | | | | TGGAATGGGTTGCCATGTTTTACGGTGATT | |
| | | | | | CTGGTTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTCAGACCGAATCTGCA | |
| | | | | | GTTGATGTTTGGGGTCAGGGCACC | |
| GAMMA_ | SSYYAYS | 293 | KPGKAPKLLIYSASFLYSGVPSRFSG | 294 | AAACCGGGTAAAGCACCGAAACTGCTGAT | 295 |
| 7 | AMDY | | SRSGTDFTLTISSLQPEDFATYYCQQ | | TTATAGCGCCTCTTTTCTGTATAGCGGTGTG | |
| | | | YYYHPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVFRSGMHWVRQA | | CAGCCGGAAGATTTTGCAACCTATTATTGC | |
| | | | PGKGLEWVASLQDDSGYTYYADSV | | CAGCAA TACTACTACCATCCTATCACCTTT | |
| | | | KGRFTISADTSKNTAYLQMNSLRAE | | GGCCAGGGCACCAAAGTTGAAATTAAACG | |
| | | | DTAVYYCSRSSYYAYSAMDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTTTAGATCTGGTATGC | |
| | | | | | ATTGGGTGCGCCAGGCACCTGGTAAAGGTC | |
| | | | | | TGGAATGGGTTGCCTCTCTGCAGGATGATT | |
| | | | | | CTGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTTCTTACTACGCATACT | |
| | | | | | CTGCAATGGATTACTGGGGTCAGGGCACC | |
| GAMMA_ | ASYIYAF | 296 | KPGKAPKLLIYSASFLYSGVPSRFSG | 297 | AAACCGGGTAAAGCACCGAAACTGCTGAT | 298 |
| 8 | DY | | SRSGTDFTLTISSLQPEDFATYYCQQ | | TTATAGCGCCTCTTTTCTGTATAGCGGTGTG | |
| | | | NYYYPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIMYEQMHWVRQ | | CAGCCGGAAGATTTTGCAACCTATTATTGC | |
| | | | APGKGLEWVAIIAPLDDYTNYADS | | CAGCAA AACTACTACTACCCTGTTACCTTT | |
| | | | VKGRFTISADTSKNTAYLQMNSLRA | | GGCCAGGGCACCAAAGTTGAAATTAAACG | |
| | | | EDTAVYYCSRASYIYAFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCATGTACGAACAGATGC | |
| | | | | | ATTGGGTGCGCCAGGCACCTGGTAAAGGTC | |
| | | | | | TGGAATGGGTTGCCATCATCGCACCACTGG | |
| | | | | | ATGATTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCATCTTACATCTACGCA | |
| | | | | | TTTGATTACTGGGGTCAGGGCACC | |
| GAMMA_ | GTGYAF | 299 | KPGKAPKLLIYSASFLYSGVPSRFSG | 300 | AAACCGGGTAAAGCACCGAAACTGCTGAT | 301 |
| 9 | DY | | SRSGTDFTLTISSLQPEDFATYYCQQ | | TTATAGCGCCTCTTTTCTGTATAGCGGTGTG | |
| | | | HYYVPLTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIHYNAMHWVRQ | | CAGCCGGAAGATTTTGCAACCTATTATTGC | |
| | | | APGKGLEWVAFVSPDGYYTNYADS | | CAGCAA CATTACTACGTTCCTCTGACCTTT | |
| | | | VKGRFTISADTSKNTAYLQMNSLRA | | GGCCAGGGCACCAAAGTTGAAATTAAACG | |
| | | | EDTAVYYCSRGTGYAFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCCATTACAACGCAATGC | |
| | | | | | ATTGGGTGCGCCAGGCACCTGGTAAAGGTC | |
| | | | | | TGGAATGGGTTGCCTTTGTTTCTCCAGATG | |
| | | | | | GTTACTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTACCGGTTACGCATTT | |
| | | | | | GATTACTGGGGTCAGGGCACC | |
| GAMMA_ | ARYSTQ | 302 | KPGKAPKLLIYSASFLYSGVPSRFSG | 303 | AAACCGGGTAAAGCACCGAAACTGCTGAT | 304 |
| 10 | YAFDY | | SRSGTDFTLTISSLQPEDFATYYCQQ | | TTATAGCGCCTCTTTTCTGTATAGCGGTGTG | |
| | | | SYSSPLTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIMPEGIHWVRQAP | | CAGCCGGAAGATTTTGCAACCTATTATTGC | |
| | | | GKGLEWVAWIWYDSDYTNYADSV | | CAGCAA TCTTACTCTTCTCCTCTGACCTTT | |
| | | | KGRFTISADTSKNTAYLQMNSLRAE | | GGCCAGGGCACCAAAGTTGAAATTAAACG | |
| | | | DTAVYYCSRARYSTQYAFDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCATGCCAGAAGGTATCC | |
| | | | | | ATTGGGTGCGCCAGGCACCTGGTAAAGGTC | |
| | | | | | TGGAATGGGTTGCCTGGATCTGGTACGATT | |
| | | | | | CTGATTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAAGATACTCTACCCAG | |
| | | | | | TACGCATTTGATTACTGGGGTCAGGGCACC | |
| GAMMA_ | DWASTS | 305 | KPGKAPKLLIYSASFLYSGVPSRFSG | 306 | AAACCGGGTAAAGCACCGAAACTGCTGAT | 307 |
| 11 | GYHYLD | | SRSGTDFTLTISSLQPEDFATYYCQQ | | TTATAGCGCCTCTTTTCTGTATAGCGGTGTG | |
| | Y | | GDSYPHTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISWTGMHWVRQA | | CAGCCGGAAGATTTTGCAACCTATTATTGC | |
| | | | PGKGLEWVAWISDGSSWTYYADSV | | CAGCAA GGTGATTCTTACCCTCATACCTTT | |
| | | | KGRFTISADTSKNTAYLQMNSLRAE | | GGCCAGGGCACCAAAGTTGAAATTAAACG | |
| | | | DTAVYYCSRDWASTSGYHYLDYW | | TACCGGTGGTGGTTCAGGTGGCGGTGGCAG | |
| | | | GQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTGGACCGGTATGC | |
| | | | | | ATTGGGTGCGCCAGGCACCTGGTAAAGGTC | |
| | | | | | TGGAATGGGTTGCCTGGATCTCTGATGGTT | |
| | | | | | CTTCTTGGACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGATTGGGCATCTACCTCT | |
| | | | | | GGTTACCATTACCTGGATTACTGGGGTCAG | |
| | | | | | GGCACC | |
| GAMM_ | YEPGYA | 308 | KPGKAPKLLIYSASFLYSGVPSRFSG | 309 | AAACCGGGTAAAGCACCGAAACTGCTGAT | 310 |
| 12 | LDY | | SRSGTDFTLTISSLQPEDFATYYCQQ | | TTATAGCGCCTCTTTTCTGTATAGCGGTGTG | |
| | | | SYYYPFTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNIRHYDMHWVRQA | | CAGCCGGAAGATTTTGCAACCTATTATTGC | |
| | | | PGKGLEWVAHILPYPGYTYYADSV | | CAGCAA TCTTACTACTACCCTTTTACCTTT | |
| | | | KGRFTISADTSKNTAYLQMNSLRAE | | GGCCAGGGCACCAAAGTTGAAATTAAACG | |
| | | | DTAVYYCSRYEPGYALDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCAGACATTACGATATGC | |
| | | | | | ATTGGGTGCGCCAGGCACCTGGTAAAGGTC | |
| | | | | | TGGAATGGGTTGCCCATATCCTGCCATACC | |
| | | | | | CTGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTACGAACCAGGTTACGCA | |
| | | | | | CTGGATTACTGGGGTCAGGGCACC | |
| GAMMA_ | SKSYFEG | 311 | KPGKAPKLLIYSASFLYSGVPSRFSG | 312 | AAACCGGGTAAAGCACCGAAACTGCTGAT | 313 |
| 13 | AMDY | | SRSGTDFTLTISSLQPEDFATYYCQQ | | TTATAGCGCCTCTTTTCTGTATAGCGGTGTG | |
| | | | GYYSPLTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSKSAMHWVRQA | | CAGCCGGAAGATTTTGCAACCTATTATTGC | |
| | | | PGKGLEWVAWIYPDGGYTNYADS | | CAGCAA GGTTACTACTCTCCTCTGACCTTT | |
| | | | VKGRFTISADTSKNTAYLQMNSLRA | | GGCCAGGGCACCAAAGTTGAAATTAAACG | |
| | | | EDTAVYYCSRSKSYFEGAMDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTAAATCTGCAATGC | |
| | | | | | ATTGGGTGCGCCAGGCACCTGGTAAAGGTC | |
| | | | | | TGGAATGGGTTGCCTGGATCTACCCAGATG | |
| | | | | | GTGGTTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTAAATCTTACTTTGAA | |
| | | | | | GGTGCAATGGATTACTGGGGTCAGGGCAC | |
| | | | | | C | |
| GAMMA_ | DMLYED | 314 | KPGKAPKLLIYSASFLYSGVPSRFSG | 315 | AAACCGGGTAAAGCACCGAAACTGCTGAT | 316 |
| 14 | GFDV | | SRSGTDFTLTISSLQPEDFATYYCQQ | | TTATAGCGCCTCTTTTCTGTATAGCGGTGTG | |
| | | | AGYSPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNFYQYGMHWVRQA | | CAGCCGGAAGATTTTGCAACCTATTATTGC | |
| | | | PGKGLEWVAFIWPGSSYTYYADSV | | CAGCAA GCAGGTTACTCTCCTATCACCTTT | |
| | | | KGRFTISADTSKNTAYLQMNSLRAE | | GGCCAGGGCACCAAAGTTGAAATTAAACG | |
| | | | DTAVYYCSRDMLYEDGFDVWGQG | | TACCGGTGGTGGTTCAGGTGGCGGTGGCAG | |
| | | | T | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACTTTTACCAGTACGGTATGC | |
| | | | | | ATTGGGTGCGCCAGGCACCTGGTAAAGGTC | |
| | | | | | TGGAATGGGTTGCCTTTATCTGGCCAGGTT | |
| | | | | | CTTCTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGATATGCTGTACGAAGAT | |
| | | | | | GGTTTTGATGTTTGGGGTCAGGGCACC | |
| GAMMA_ | HMGDA | 317 | KPGKAPKLLIYSASFLYSGVPSRFSG | 318 | AAACCGGGTAAAGCACCGAAACTGCTGAT | 319 |
| 15 | MDY | | SRSGTDFTLTISSLQPEDFATYYCQQ | | TTATAGCGCCTCTTTTCTGTATAGCGGTGTG | |
| | | | NKSVPVTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNLSGYGMHWVRQA | | CAGCCGGAAGATTTTGCAACCTATTATTGC | |
| | | | PGKGLEWVAHILPWSGNTYYADSV | | CAGCAA AACAAATCTGTTCCTGTTACCTTT | |
| | | | KGRFTISADTSKNTAYLQMNSLRAE | | GGCCAGGGCACCAAAGTTGAAATTAAACG | |
| | | | DTAVYYCSRHMGDAMDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACCTGTCTGGTTACGGTATGC | |
| | | | | | ATTGGGTGCGCCAGGCACCTGGTAAAGGTC | |
| | | | | | TGGAATGGGTTGCCCATATCCTGCCATGGT | |
| | | | | | CTGGTAACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCCATATGGGTGATGCAATG | |
| | | | | | GATTACTGGGGTCAGGGCACC | |
| GAMMA_ | YHWSNY | 320 | KPGKAPKLLIYSASFLYSGVPSRFSG | 321 | AAACCGGGTAAAGCACCGAAACTGCTGAT | 322 |
| 16 | YAFDV | | SRSGTDFTLTISSLQPEDFATYYCQQ | | TTATAGCGCCTCTTTTCTGTATAGCGGTGTG | |
| | | | LSAPPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVYTYDMHWVRQ | | CAGCCGGAAGATTTTGCAACCTATTATTGC | |
| | | | APGKGLEWVADVYPWSYYTNYAD | | CAGCAA CTGTCTGCACCTCCTATCACCTTT | |
| | | | SVKGRFTISADTSKNTAYLQMNSLR | | GGCCAGGGCACCAAAGTTGAAATTAAACG | |
| | | | AEDTAVYYCSRYHWSNYYAFDVW | | TACCGGTGGTGGTTCAGGTGGCGGTGGCAG | |
| | | | GQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACACCTACGATATGC | |
| | | | | | ATTGGGTGCGCCAGGCACCTGGTAAAGGTC | |
| | | | | | TGGAATGGGTTGCCGATGTTTACCCATGGT | |
| | | | | | CTTACTACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTACCATTGGTCTAACTAC | |
| | | | | | TACGCATTTGATGTTTGGGGTCAGGGCACC | |
| GAMMA_ | EMAHDS | 323 | KPGKAPKLLIYSAYFLYSGVPSRFSG | 324 | AAACCGGGTAAAGCACCGAAACTGCTGAT | 325 |
| 17 | YAMDV | | SRSGTDFTLTISSLQPEDFATYYCQQ | | TTATAGCGCCTACTTTCTGTATAGCGGTGT | |
| | | | YTSSPITFGQGTKVEIKRTGGGSGG | | GCCGAGCCGTTTTAGCGGTAGCCGTAGCGG | |
| | | | GGSGGGASEVQLVESGGGLVQPGG | | CACCGATTTTACCCTGACCATTAGCAGCCT | |
| | | | SLRLSCAASGFNFSRQIMHWVRQAP | | GCAGCCGGAAGATTTTGCAACCTATTATTG | |
| | | | GKGLEWVAWIEGSSGNTNYADSVK | | CCAGCAA TACACCTCTTCTCCTATCACCTT | |
| | | | GRFTISADTSKNTAYLQMNSLRAED | | TGGCCAGGGCACCAAAGTTGAAATTAAAC | |
| | | | TAVYYCSREMAHDSYAMDVWGQ | | GTACCGGTGGTGGTTCAGGTGGCGGTGGCA | |
| | | | GT | | GCGGAGGCGGAGCCAGCGAAGTGCAGCTG | |
| | | | | | GTTGAAAGCGGTGGCGGTCTGGTTCAGCCA | |
| | | | | | GGTGGTAGCCTGCGTCTGAGCTGTGCAGCA | |
| | | | | | AGCGGTTTTAACTTTTCTAGACAGATCATG | |
| | | | | | CATTGGGTGCGCCAGGCACCTGGTAAAGGT | |
| | | | | | CTGGAATGGGTTGCCTGGATCGAAGGTTCT | |
| | | | | | TCTGGTAACACCAACTATGCCGATAGCGTG | |
| | | | | | AAAGGTCGCTTTACCATTAGCGCAGATACC | |
| | | | | | AGCAAAAATACCGCATACCTGCAGATGAA | |
| | | | | | TAGTCTGCGTGCAGAGGATACAGCAGTGTA | |
| | | | | | TTATTGTAGCCGCGAAATGGCACATGATTC | |
| | | | | | TTACGCAATGGATGTTTGGGGTCAGGGCAC | |
| | | | | | C | |
| GAMMA_ | QYYQEA | 326 | KPGKAPKLLIYSASFLYSGVPSRFSG | 327 | AAACCGGGTAAAGCACCGAAACTGCTGAT | 328 |
| 18 | FDY | | SRSGTDFTLTISSLQPEDFATYYCQQ | | TTATAGCGCCTCTTTTCTGTATAGCGGTGTG | |
| | | | YSYYGPITFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNISSQYIHWVRQA | | CAGCCGGAAGATTTTGCAACCTATTATTGC | |
| | | | PGKGLEWVATLYPSSGNTPYADSV | | CAGCAA TACTCTTACTACGGTCCTATCACC | |
| | | | KGRFTISADTSKNTAYLQMNSLRAE | | TTTGGCCAGGGCACCAAAGTTGAAATTAAA | |
| | | | DTAVYYCSRQYYQEAFDYWGQGT | | CGTACCGGTGGTGGTTCAGGTGGCGGTGGC | |
| | | | | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCTCTTCTCAGTACAT | |
| | | | | | CCATTGGGTGCGCCAGGCACCTGGTAAAG | |
| | | | | | GTCTGGAATGGGTTGCCACCCTGTACCCAT | |
| | | | | | CTTCTGGTAACACCCCATATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCCAGTACTACCAGGA | |
| | | | | | AGCATTTGATTACTGGGGTCAGGGCACC | |
| GAMMA_ | VASTAF | 329 | KPGKAPKLLIYSASFLYSGVPSRFSG | 330 | AAACCGGGTAAAGCACCGAAACTGCTGAT | 331 |
| 19 | DY | | SRSGTDFTLTISSLQPEDFATYYCQQ | | TTATAGCGCCTCTTTTCTGTATAGCGGTGTG | |
| | | | TRWYPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNFSSIPMHWVRQA | | CAGCCGGAAGATTTTGCAACCTATTATTGC | |
| | | | PGKGLEWVADIYGSSGHTNYADSV | | CAGCAA ACCAGATGGTACCCTGTTACCTTT | |
| | | | KGRFTISADTSKNTAYLQMNSLRAE | | GGCCAGGGCACCAAAGTTGAAATTAAACG | |
| | | | DTAVYYCSRVASTAFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACTTTTCTTCTATCCCAATGCA | |
| | | | | | TTGGGTGCGCCAGGCACCTGGTAAAGGTCT | |
| | | | | | GGAATGGGTTGCCGATATCTACGGTTCTTC | |
| | | | | | TGGTCATACCAACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGTTGCATCTACCGCATTTG | |
| | | | | | ATTACTGGGGTCAGGGCACC | |
| GAMMA_ | ANLGYIS | 332 | KPGKAPKLLIYSASFLYSGVPSRFSG | 333 | AAACCGGGTAAAGCACCGAAACTGCTGAT | 334 |
| 20 | YMDY | | SRSGTDFTLTISSLQPEDFATYYCQQ | | TTATAGCGCCTCTTTTCTGTATAGCGGTGTG | |
| | | | GYFEPYTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSKTGMHWVRQA | | CAGCCGGAAGATTTTGCAACCTATTATTGC | |
| | | | PGKGLEWVALIWPSMGYTSYADSV | | CAGCAA GGTTACTTTGAACCTTACACCTTT | |
| | | | KGRFTISADTSKNTAYLQMNSLRAE | | GGCCAGGGCACCAAAGTTGAAATTAAACG | |
| | | | DTAVYYCSRANLGYISYMDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTAAAACCGGTATGC | |
| | | | | | ATTGGGTGCGCCAGGCACCTGGTAAAGGTC | |
| | | | | | TGGAATGGGTTGCCCTGATCTGGCCATCTA | |
| | | | | | TGGGTTACACCTCTTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAAACCTGGGTTACATC | |
| | | | | | TCTTACATGGATTACTGGGGTCAGGGCACC | |
| DELTA_ | SYQSPSG | 335 | KPGKAPKLLIYSASFLYSGVPSRFSG | 336 | AAACCGGGTAAAGCACCGAAACTGCTGAT | 337 |
| 1 | SYYYHA | | SRSGTDFTLTISSLQPEDFATYYCQQ | | TTATAGCGCCTCTTTTCTGTATAGCGGTGTG | |
| | FDY | | STTAPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSYYGMHWVRQA | | CAGCCGGAAGATTTTGCAACCTATTATTGC | |
| | | | PGKGLEWVAAVVGDSDYTYYADS | | CAGCAA TCTACCACCGCACCTATCACCTTT | |
| | | | VKGRFTISADTSKNTAYLQMNSLRA | | GGCCAGGGCACCAAAGTTGAAATTAAACG | |
| | | | EDTAVYYCSRSYQSPSGSYYYHAF | | TACCGGTGGTGGTTCAGGTGGCGGTGGCAG | |
| | | | DYWGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTTACTACGGTATGC | |
| | | | | | ATTGGGTGCGCCAGGCACCTGGTAAAGGTC | |
| | | | | | TGGAATGGGTTGCCGCAGTTGTTGGTGATT | |
| | | | | | CTGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTTACCAGTCTCCATCTG | |
| | | | | | GTTCTTACTACTACCATGCATTTGATTACTG | |
| | | | | | GGGTCAGGGCACC | |
| DELTA_ | SDGSSYA | 338 | KPGKAPKLLIYSASFLYSGVPSRFSG | 339 | AAACCGGGTAAAGCACCGAAACTGCTGAT | 340 |
| 2 | LDY | | SRSGTDFTLTISSLQPEDFATYYCQQ | | TTATAGCGCCTCTTTTCTGTATAGCGGTGTG | |
| | | | KYYRPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIYNQDMHWVRQ | | CAGCCGGAAGATTTTGCAACCTATTATTGC | |
| | | | APGKGLEWVAELWGYSDHTNYAD | | CAGCAA AAATACTACAGACCTGTTACCTTT | |
| | | | SVKGRFTISADTSKNTAYLQMNSLR | | GGCCAGGGCACCAAAGTTGAAATTAAACG | |
| | | | AEDTAVYYCSRSDGSSYALDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACAACCAGGATATGC | |
| | | | | | ATTGGGTGCGCCAGGCACCTGGTAAAGGTC | |
| | | | | | TGGAATGGGTTGCCGAACTGTGGGGTTACT | |
| | | | | | CTGATCATACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTGATGGTTCTTCTTACG | |
| | | | | | CACTGGATTACTGGGGTCAGGGCACC | |
| DELTA_ | SYDVYA | 341 | KPGKAPKLLIYSASFLYSGVPSRFSG | 342 | AAACCGGGTAAAGCACCGAAACTGCTGAT | 343 |
| 3 | FDY | | SRSGTDFTLTISSLQPEDFATYYCQQ | | TTATAGCGCCTCTTTTCTGTATAGCGGTGTG | |
| | | | SWEMPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVYAYQMHWVR | | CAGCCGGAAGATTTTGCAACCTATTATTGC | |
| | | | QAPGKGLEWVAFISDDSGNTNYAD | | CAGCAA TCTTGGGAAATGCCTGTTACCTTT | |
| | | | SVKGRFTISADTSKNTAYLQMNSLR | | GGCCAGGGCACCAAAGTTGAAATTAAACG | |
| | | | AEDTAVYYCSRSYDVYAFDYWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACGCATACCAGATGC | |
| | | | | | ATTGGGTGCGCCAGGCACCTGGTAAAGGTC | |
| | | | | | TGGAATGGGTTGCCTTTATCTCTGATGATT | |
| | | | | | CTGGTAACACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCTCTTACGATGTTTACGCA | |
| | | | | | TTTGATTACTGGGGTCAGGGCACC | |
| DELTA_ | GDSSYSY | 344 | KPGKAPKLLIYSASFLYSGVPSRFSG | 345 | AAACCGGGTAAAGCACCGAAACTGCTGAT | 346 |
| 4 | HPYYSSA | | SRSGTDFTLTISSLQPEDFATYYCQQ | | TTATAGCGCCTCTTTTCTGTATAGCGGTGTG | |
| | MDY | | SGYSTPWTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNLSQYKMHWVRQ | | CAGCCGGAAGATTTTGCAACCTATTATTGC | |
| | | | APGKGLEWVAAIDGYSDNTNYADS | | CAGCAA TCTGGTTACTCTACCCCTTGGACC | |
| | | | VKGRFTISADTSKNTAYLQMNSLRA | | TTTGGCCAGGGCACCAAAGTTGAAATTAAA | |
| | | | EDTAVYYCSRGDSSYSYHPYYSSA | | CGTACCGGTGGTGGTTCAGGTGGCGGTGGC | |
| | | | MDYWGQGT | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACCTGTCTCAGTACAAAAT | |
| | | | | | GCATTGGGTGCGCCAGGCACCTGGTAAAG | |
| | | | | | GTCTGGAATGGGTTGCCGCAATCGATGGTT | |
| | | | | | ACTCTGATAACACCAACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCGGTGATTCTTCTTAC | |
| | | | | | TCTTACCATCCATACTACTCTTCTGCAATGG | |
| | | | | | ATTACTGGGGTCAGGGCACC | |
| DELTA_ | NEMGSK | 347 | KPGKAPKLLIYSASFLYSGVPSRFSG | 348 | AAACCGGGTAAAGCACCGAAACTGCTGAT | 349 |
| 5 | LSGFDY | | SRSGTDFTLTISSLQPEDFATYYCQQ | | TTATAGCGCCTCTTTTCTGTATAGCGGTGTG | |
| | | | SYYYPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISYDHMHWVRQA | | CAGCCGGAAGATTTTGCAACCTATTATTGC | |
| | | | PGKGLEWVATLYPSGGFTNYADSV | | CAGCAA TCTTACTACTACCCTATCACCTTT | |
| | | | KGRFTISADTSKNTAYLQMNSLRAE | | GGCCAGGGCACCAAAGTTGAAATTAAACG | |
| | | | DTAVYYCSRNEMGSKLSGFDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTACGATCATATGC | |
| | | | | | ATTGGGTGCGCCAGGCACCTGGTAAAGGTC | |
| | | | | | TGGAATGGGTTGCCACCCTGTACCCATCTG | |
| | | | | | GTGGTTTTACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCAACGAAATGGGTTCTAAA | |
| | | | | | CTGTCTGGTTTTGATTACTGGGGTCAGGGC | |
| | | | | | ACC | |
| DELTA_ | NELCSK | 350 | KPGKAPKLLIYSASFLYSGVPSRFSG | 351 | AAACCGGGTAAAGCACCGAAACTGCTGAT | 352 |
| 6 | LSGFDY | | SRSGTDFTLTISSLQPEDFATYYCQQ | | TTATAGCGCCTCTTTTCTGTATAGCGGTGTG | |
| | | | SYYYPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISYDHMHWVRQA | | CAGCCGGAAGATTTTGCAACCTATTATTGC | |
| | | | PGKGLEWVATLYPSGGFTNYADSV | | CAGCAA TCTTACTACTACCCTATCACCTTT | |
| | | | KGRFTISADTSKNTAYLQMNSLRAE | | GGCCAGGGCACCAAAGTTGAAATTAAACG | |
| | | | DTAVYYCSRNELCSKLSGFDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTACGATCATATGC | |
| | | | | | ATTGGGTGCGCCAGGCACCTGGTAAAGGTC | |
| | | | | | TGGAATGGGTTGCCACCCTGTACCCATCTG | |
| | | | | | GTGGTTTTACCAACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCAACGAACTGTGTTCTAAA | |
| | | | | | CTGTCTGGTTTTGATTACTGGGGTCAGGGC | |
| | | | | | ACC | |
| DELTA_ | GQFYYS | 353 | KPGKAPKLLIYSASFLYSGVPSRFSG | 354 | AAACCGGGTAAAGCACCGAAACTGCTGAT | 355 |
| 7 | SYMGSY | | SRSGTDFTLTISSLQPEDFATYYCQQ | | TTATAGCGCCTCTTTTCTGTATAGCGGTGTG | |
| | SYYNAF | | QYSPITFGQGTKVEIKRTGGGSGGG | | CCGAGCCGTTTTAGCGGTAGCCGTAGCGGC | |
| | DY | | GSGGGASEVQLVESGGGLVQPGGS | | ACCGATTTTACCCTGACCATTAGCAGCCTG | |
| | | | LRLSCAASGFNIYSWQIHWVRQAPG | | CAGCCGGAAGATTTTGCAACCTATTATTGC | |
| | | | KGLEWVAHLSYDGGNTYYADSVK | | CAGCAA CAGTACTCTCCTATCACCTTTGGC | |
| | | | GRFTISADTSKNTAYLQMNSLRAED | | CAGGGCACCAAAGTTGAAATTAAACGTAC | |
| | | | TAVYYCSRGQFYYSSYMGSYSYY | | CGGTGGTGGTTCAGGTGGCGGTGGCAGCG | |
| | | | NAFDYWGQGT | | GAGGCGGAGCCAGCGAAGTGCAGCTGGTT | |
| | | | | | GAAAGCGGTGGCGGTCTGGTTCAGCCAGG | |
| | | | | | TGGTAGCCTGCGTCTGAGCTGTGCAGCAAG | |
| | | | | | CGGTTTTAACATCTACTCTTGGCAGATCCA | |
| | | | | | TTGGGTGCGCCAGGCACCTGGTAAAGGTCT | |
| | | | | | GGAATGGGTTGCCCATCTGTCTTACGATGG | |
| | | | | | TGGTAACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGGTCAGTTTTACTACTCTTC | |
| | | | | | TTACATGGGTTCTTACTCTTACTACAACGC | |
| | | | | | ATTTGATTACTGGGGTCAGGGCACC | |
| DELTA_ | AVYPYA | 356 | KPGKAPKLLIYSASFLYSGVPSRFSG | 357 | AAACCGGGTAAAGCACCGAAACTGCTGAT | 358 |
| 8 | MDV | | SRSGTDFTLTISSLQPEDFATYYCQQ | | TTATAGCGCCTCTTTTCTGTATAGCGGTGTG | |
| | | | SMSYPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVYAYGMHWVRQ | | CAGCCGGAAGATTTTGCAACCTATTATTGC | |
| | | | APGKGLEWVARFYDQDGYTYYAD | | CAGCAA TCTATGTCTTACCCTATCACCTTT | |
| | | | SVKGRFTISADTSKNTAYLQMNSLR | | GGCCAGGGCACCAAAGTTGAAATTAAACG | |
| | | | AEDTAVYYCSRAVYPYAMDVWGQ | | TACCGGTGGTGGTTCAGGTGGCGGTGGCAG | |
| | | | GT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACGCATACGGTATGC | |
| | | | | | ATTGGGTGCGCCAGGCACCTGGTAAAGGTC | |
| | | | | | TGGAATGGGTTGCCAGATTTTACGATCAGG | |
| | | | | | ATGGTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAGTTTACCCATACGCA | |
| | | | | | ATGGATGTTTGGGGTCAGGGCACC | |
| DELTA_ | NYSYYA | 359 | KPGKAPKLLIYSASFLYSGVPSRFSG | 360 | AAACCGGGTAAAGCACCGAAACTGCTGAT | 361 |
| 9 | FDV | | SRSGTDFTLTISSLQPEDFATYYCQQ | | TTATAGCGCCTCTTTTCTGTATAGCGGTGTG | |
| | | | VTVGSPTTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVYQYGMHWVR | | CAGCCGGAAGATTTTGCAACCTATTATTGC | |
| | | | QAPGKGLEWVAKVSDQDGSTYYA | | CAGCAA GTTACCGTTGGTTCTCCTACCACC | |
| | | | DSVKGRFTISADTSKNTAYLQMNSL | | TTTGGCCAGGGCACCAAAGTTGAAATTAAA | |
| | | | RAEDTAVYYCSRNYSYYAFDVWG | | CGTACCGGTGGTGGTTCAGGTGGCGGTGGC | |
| | | | QGT | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACGTTTACCAGTACGGTAT | |
| | | | | | GCATTGGGTGCGCCAGGCACCTGGTAAAG | |
| | | | | | GTCTGGAATGGGTTGCCAAAGTTTCTGATC | |
| | | | | | AGGATGGTTCTACCTACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCAACTACTCTTACTA | |
| | | | | | CGCATTTGATGTTTGGGGTCAGGGCACC | |
| DELTA_ | HLYMYF | 362 | KPGKAPKLLIYSASFLYSGVPSRFSG | 363 | AAACCGGGTAAAGCACCGAAACTGCTGAT | 364 |
| 10 | DY | | SRSGTDFTLTISSLQPEDFATYYCQQ | | TTATAGCGCCTCTTTTCTGTATAGCGGTGTG | |
| | | | MSSAPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNFMYYGMHWVRQ | | CAGCCGGAAGATTTTGCAACCTATTATTGC | |
| | | | APGKGLEWVAHIFPGDSYTYYADS | | CAGCAA ATGTCTTCTGCACCTATCACCTTT | |
| | | | VKGRFTISADTSKNTAYLQMNSLRA | | GGCCAGGGCACCAAAGTTGAAATTAAACG | |
| | | | EDTAVYYCSRHLYMYFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACTTTATGTACTACGGTATGC | |
| | | | | | ATTGGGTGCGCCAGGCACCTGGTAAAGGTC | |
| | | | | | TGGAATGGGTTGCCCATATCTTTCCAGGTG | |
| | | | | | ATTCTTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCCATCTGTACATGTACTTT | |
| | | | | | GATTACTGGGGTCAGGGCACC | |
| DELTA_ | ADYYYS | 365 | KPGKAPKLLIYSASFLYSGVPSRFSG | 366 | AAACCGGGTAAAGCACCGAAACTGCTGAT | 367 |
| 11 | HSYYYSS | | SRSGTDFTLTISSLQPEDFATYYCQQ | | TTATAGCGCCTCTTTTCTGTATAGCGGTGTG | |
| | SYLDV | | AGYYPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIYSYDMHWVRQ | | CAGCCGGAAGATTTTGCAACCTATTATTGC | |
| | | | APGKGLEWVAAISPDDDYTYYADS | | CAGCAA GCAGGTTACTACCCTGTTACCTTT | |
| | | | VKGRFTISADTSKNTAYLQMNSLRA | | GGCCAGGGCACCAAAGTTGAAATTAAACG | |
| | | | EDTAVYYCSRADYYYSHSYYYSSS | | TACCGGTGGTGGTTCAGGTGGCGGTGGCAG | |
| | | | YLDVWGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACTCTTACGATATGC | |
| | | | | | ATTGGGTGCGCCAGGCACCTGGTAAAGGTC | |
| | | | | | TGGAATGGGTTGCCGCAATCTCTCCAGATG | |
| | | | | | ATGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAGATTACTACTACTCT | |
| | | | | | CACTCTTACTACTACTCTTCTTCTTACCTGG | |
| | | | | | ATGTTTGGGGTCAGGGCACC | |
| DELTA_ | YSSQSYY | 368 | KPGKAPKLLIYSASFLYSGVPSRFSG | 369 | AAACCGGGTAAAGCACCGAAACTGCTGAT | 370 |
| 12 | MDY | | SRSGTDFTLTISSLQPEDFATYYCQQ | | TTATAGCGCCTCTTTTCTGTATAGCGGTGTG | |
| | | | QYDTPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISSYGIHWVRQAP | | CAGCCGGAAGATTTTGCAACCTATTATTGC | |
| | | | GKGLEWVAFFYPDSSYTYYADSVK | | CAGCAA CAGTACGATACCCCTATCACCTTT | |
| | | | GRFTISADTSKNTAYLQMNSLRAED | | GGCCAGGGCACCAAAGTTGAAATTAAACG | |
| | | | TAVYYCSRYSSQSYYMDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTTCTTACGGTATCCA | |
| | | | | | TTGGGTGCGCCAGGCACCTGGTAAAGGTCT | |
| | | | | | GGAATGGGTTGCCTTTTTTTACCCAGATTCT | |
| | | | | | TCTTACACCTACTATGCCGATAGCGTGAAA | |
| | | | | | GGTCGCTTTACCATTAGCGCAGATACCAGC | |
| | | | | | AAAAATACCGCATACCTGCAGATGAATAG | |
| | | | | | TCTGCGTGCAGAGGATACAGCAGTGTATTA | |
| | | | | | TTGTAGCCGCTACTCTTCTCAGTCTTACTAC | |
| | | | | | ATGGATTACTGGGGTCAGGGCACC | |
| DELTA_ | ELYEYA | 371 | KPGKAPKLLIYSASFLYSGVPSRFSG | 372 | AAACCGGGTAAAGCACCGAAACTGCTGAT | 373 |
| 13 | FDY | | SRSGTDFTLTISSLQPEDFATYYCQQ | | TTATAGCGCCTCTTTTCTGTATAGCGGTGTG | |
| | | | YSYPVTFGQGTKVEIKRTGGGSGGG | | CCGAGCCGTTTTAGCGGTAGCCGTAGCGGC | |
| | | | GSGGGASEVQLVESGGGLVQPGGS | | ACCGATTTTACCCTGACCATTAGCAGCCTG | |
| | | | LRLSCAASGFNVTASNMHWVRQAP | | CAGCCGGAAGATTTTGCAACCTATTATTGC | |
| | | | GKGLEWVAIVSPDSGYTYYADSVK | | CAGCAA TACTCTTACCCTGTTACCTTTGGC | |
| | | | GRFTISADTSKNTAYLQMNSLRAED | | CAGGGCACCAAAGTTGAAATTAAACGTAC | |
| | | | TAVYYCSRELYEYAFDYWGQGT | | CGGTGGTGGTTCAGGTGGCGGTGGCAGCG | |
| | | | | | GAGGCGGAGCCAGCGAAGTGCAGCTGGTT | |
| | | | | | GAAAGCGGTGGCGGTCTGGTTCAGCCAGG | |
| | | | | | TGGTAGCCTGCGTCTGAGCTGTGCAGCAAG | |
| | | | | | CGGTTTTAACGTTACCGCATCTAACATGCA | |
| | | | | | TTGGGTGCGCCAGGCACCTGGTAAAGGTCT | |
| | | | | | GGAATGGGTTGCCATCGTTTCTCCAGATTC | |
| | | | | | TGGTTACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCGAACTGTACGAATACGCAT | |
| | | | | | TTGATTACTGGGGTCAGGGCACC | |
| DELTA | SYGNYA | 374 | KPGKAPKLLIYSASFLYSGVPSRFSG | 375 | AAACCGGGTAAAGCACCGAAACTGCTGAT | 376 |
| 14 | FDY | | SRSGTDFTLTISSLQPEDFATYYCQQ | | TTATAGCGCCTCTTTTCTGTATAGCGGTGTG | |
| | | | TWSSSPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIYQYVIHWVRQA | | CAGCCGGAAGATTTTGCAACCTATTATTGC | |
| | | | PGKGLEWVALVTPDDGYTNYADSV | | CAGCAA ACCTGGTCTTCTTCTCCTGTTACC | |
| | | | KGRFTISADTSKNTAYLQMNSLRAE | | TTTGGCCAGGGCACCAAAGTTGAAATTAAA | |
| | | | DTAVYYCSRSYGNYAFDYWGQGT | | CGTACCGGTGGTGGTTCAGGTGGCGGTGGC | |
| | | | | | AGCGGAGGCGGAGCCAGCGAAGTGCAGCT | |
| | | | | | GGTTGAAAGCGGTGGCGGTCTGGTTCAGCC | |
| | | | | | AGGTGGTAGCCTGCGTCTGAGCTGTGCAGC | |
| | | | | | AAGCGGTTTTAACATCTACCAGTACGTTAT | |
| | | | | | CCATTGGGTGCGCCAGGCACCTGGTAAAG | |
| | | | | | GTCTGGAATGGGTTGCCCTGGTTACCCCAG | |
| | | | | | ATGATGGTTACACCAACTATGCCGATAGCG | |
| | | | | | TGAAAGGTCGCTTTACCATTAGCGCAGATA | |
| | | | | | CCAGCAAAAATACCGCATACCTGCAGATG | |
| | | | | | AATAGTCTGCGTGCAGAGGATACAGCAGT | |
| | | | | | GTATTATTGTAGCCGCTCTTACGGTAACTA | |
| | | | | | CGCATTTGATTACTGGGGTCAGGGCACC | |
| DELTA_ | ADYYYS | 377 | KPGKAPKLLIYSASFLYSGVPSRFSG | 378 | AAACCGGGTAAAGCACCGAAACTGCTGAT | 379 |
| 15 | NSYYYSS | | SRSGTDFTLTISSLQPEDFATYYCQQ | | TTATAGCGCCTCTTTTCTGTATAGCGGTGTG | |
| | SYLDV | | AGYYPVTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNIYSYDMHWVRQ | | CAGCCGGAAGATTTTGCAACCTATTATTGC | |
| | | | APGKGLEWVAAISPDDDYTYYADS | | CAGCAA GCAGGTTACTACCCTGTTACCTTT | |
| | | | VKGRFTISADTSKNTAYLQMNSLRA | | GGCCAGGGCACCAAAGTTGAAATTAAACG | |
| | | | EDTAVYYCSRADYYYSNSYYYSSS | | TACCGGTGGTGGTTCAGGTGGCGGTGGCAG | |
| | | | YLDVWGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTACTCTTACGATATGC | |
| | | | | | ATTGGGTGCGCCAGGCACCTGGTAAAGGTC | |
| | | | | | TGGAATGGGTTGCCGCAATCTCTCCAGATG | |
| | | | | | ATGATTACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGCAGATTACTACTACTCT | |
| | | | | | AACTCTTACTACTACTCTTCTTCTTACCTGG | |
| | | | | | ATGTTTGGGGTCAGGGCACC | |
| DELTA_ | SYMTYA | 380 | KPGKAPKLLIYSASFLYSGVPSRFSG | 381 | AAACCGGGTAAAGCACCGAAACTGCTGAT | 382 |
| 16 | FDY | | SRSGTDFTLTISSLQPEDFATYYCQQ | | TTATAGCGCCTCTTTTCTGTATAGCGGTGTG | |
| | | | STYKPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNLYSYSIHWVRQAP | | CAGCCGGAAGATTTTGCAACCTATTATTGC | |
| | | | GKGLEWVAAINSDDGYTNYADSVK | | CAGCAA TCTACCTACAAACCTATCACCTTT | |
| | | | GRFTISADTSKNTAYLQMNSLRAED | | GGCCAGGGCACCAAAGTTGAAATTAAACG | |
| | | | TAVYYCSRSYMTYAFDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACCTGTACTCTTACTCTATCCA | |
| | | | | | TTGGGTGCGCCAGGCACCTGGTAAAGGTCT | |
| | | | | | GGAATGGGTTGCCGCGATCAACTCTGATGA | |
| | | | | | TGGTTACACCAACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCTCTTACATGACCTACGCAT | |
| | | | | | TTGATTACTGGGGTCAGGGCACC | |
| DELTA_ | ETSYAFS | 383 | KPGKAPKLLIYSASFLYSGVPSRFSG | 384 | AAACCGGGTAAAGCACCGAAACTGCTGAT | 385 |
| 17 | AMDY | | SRSGTDFTLTISSLQPEDFATYYCQQ | | TTATAGCGCCTCTTTTCTGTATAGCGGTGTG | |
| | | | YYYSPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVSYGDMHWVRQA | | CAGCCGGAAGATTTTGCAACCTATTATTGC | |
| | | | PGKGLEWVAKFHDGSGHTYYADS | | CAGCAA TACTACTACTCTCCTATCACCTTT | |
| | | | VKGRFTISADTSKNTAYLQMNSLRA | | GGCCAGGGCACCAAAGTTGAAATTAAACG | |
| | | | EDTAVYYCSRETSYAFSAMDYWG | | TACCGGTGGTGGTTCAGGTGGCGGTGGCAG | |
| | | | QGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTCTTACGGTGATATGC | |
| | | | | | ATTGGGTGCGCCAGGCACCTGGTAAAGGTC | |
| | | | | | TGGAATGGGTTGCCAAATTTCATGATGGTT | |
| | | | | | CTGGTCACACCTACTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGAAACCTCTTACGCATTT | |
| | | | | | TCTGCAATGGATTACTGGGGTCAGGGCACC | |
| DELTA_ | HHGGYY | 386 | KPGKAPKLLIYSASFLYSGVPSRFSG | 387 | AAACCGGGTAAAGCACCGAAACTGCTGAT | 388 |
| 18 | ALDY | | SRSGTDFTLTISSLQPEDFATYYCQQ | | TTATAGCGCCTCTTTTCTGTATAGCGGTGTG | |
| | | | FSYSPFTFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNISGSSMHWVRQAP | | CAGCCGGAAGATTTTGCAACCTATTATTGC | |
| | | | GKGLEWVAQIYSGYDYTYYADSVK | | CAGCAA TTTTCTTACTCTCCTTTTACCTTTG | |
| | | | GRFTISADTSKNTAYLQMNSLRAED | | GCCAGGGCACCAAAGTTGAAATTAAACGT | |
| | | | TAVYYCSRHHGGYYALDYWGQGT | | ACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACATCTCTGGTTCTTCTATGCA | |
| | | | | | TTGGGTGCGCCAGGCACCTGGTAAAGGTCT | |
| | | | | | GGAATGGGTTGCCCAGATCTACTCTGGTTA | |
| | | | | | CGATTACACCTACTATGCCGATAGCGTGAA | |
| | | | | | AGGTCGCTTTACCATTAGCGCAGATACCAG | |
| | | | | | CAAAAATACCGCATACCTGCAGATGAATA | |
| | | | | | GTCTGCGTGCAGAGGATACAGCAGTGTATT | |
| | | | | | ATTGTAGCCGCCATCATGGTGGTTACTACG | |
| | | | | | CACTGGATTACTGGGGTCAGGGCACC | |
| DELTA_ | GQDSHA | 389 | KPGKAPKLLIYSASFLYSGVPSRFSG | 390 | AAACCGGGTAAAGCACCGAAACTGCTGAT | 391 |
| 19 | TYYYSF | | SRSGTDFTLTISSLQPEDFATYYCQQ | | TTATAGCGCCTCTTTTCTGTATAGCGGTGTG | |
| | DV | | DHYYPMTFGQGTKVEIKRTGGGSG | | CCGAGCCGTTTTAGCGGTAGCCGTAGCGGC | |
| | | | GGGSGGGASEVQLVESGGGLVQPG | | ACCGATTTTACCCTGACCATTAGCAGCCTG | |
| | | | GSLRLSCAASGFNVGNSGMHWVRQ | | CAGCCGGAAGATTTTGCAACCTATTATTGC | |
| | | | APGKGLEWVAYVYDGSDSTEYADS | | CAGCAA GATCATTACTACCCTATGACCTTT | |
| | | | VKGRFTISADTSKNTAYLQMNSLRA | | GGCCAGGGCACCAAAGTTGAAATTAAACG | |
| | | | EDTAVYYCSRGQDSHATYYYSFDV | | TACCGGTGGTGGTTCAGGTGGCGGTGGCAG | |
| | | | WGQGT | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTGGTAACTCTGGTATGC | |
| | | | | | ATTGGGTGCGCCAGGCACCTGGTAAAGGTC | |
| | | | | | TGGAATGGGTTGCCTACGTTTACGATGGTT | |
| | | | | | CTGATTCTACCGAATATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCGGTCAGGATTCTCATGCA | |
| | | | | | ACCTACTACTACTCTTTTGATGTTTGGGGTC | |
| | | | | | AGGGCACC | |
| DELTA_ | NYSKYA | 392 | KPGKAPKLLIYSASFLYSGVPSRFSG | 393 | AAACCGGGTAAAGCACCGAAACTGCTGAT | 394 |
| _20 | MDY | | SRSGTDFTLTISSLQPEDFATYYCQQ | | TTATAGCGCCTCTTTTCTGTATAGCGGTGTG | |
| | | | YNYTPITFGQGTKVEIKRTGGGSGG | | CCGAGCCGTTTTAGCGGTAGCCGTAGCGGC | |
| | | | GGSGGGASEVQLVESGGGLVQPGG | | ACCGATTTTACCCTGACCATTAGCAGCCTG | |
| | | | SLRLSCAASGFNVYAYSMHWVRQA | | CAGCCGGAAGATTTTGCAACCTATTATTGC | |
| | | | PGKGLEWVATFSDDSGVTGYADSV | | CAGCAA TACAACTACACCCCTATCACCTTT | |
| | | | KGRFTISADTSKNTAYLQMNSLRAE | | GGCCAGGGCACCAAAGTTGAAATTAAACG | |
| | | | DTAVYYCSRNYSKYAMDYWGQGT | | TACCGGTGGTGGTTCAGGTGGCGGTGGCAG | |
| | | | | | CGGAGGCGGAGCCAGCGAAGTGCAGCTGG | |
| | | | | | TTGAAAGCGGTGGCGGTCTGGTTCAGCCAG | |
| | | | | | GTGGTAGCCTGCGTCTGAGCTGTGCAGCAA | |
| | | | | | GCGGTTTTAACGTTTACGCATACTCTATGC | |
| | | | | | ATTGGGTGCGCCAGGCACCTGGTAAAGGTC | |
| | | | | | TGGAATGGGTTGCCACCTTTTCTGATGATT | |
| | | | | | CTGGTGTTACCGGTTATGCCGATAGCGTGA | |
| | | | | | AAGGTCGCTTTACCATTAGCGCAGATACCA | |
| | | | | | GCAAAAATACCGCATACCTGCAGATGAAT | |
| | | | | | AGTCTGCGTGCAGAGGATACAGCAGTGTAT | |
| | | | | | TATTGTAGCCGCAACTACTCTAAATACGCA | |
| | | | | | ATGGATTACTGGGGTCAGGGCACC | |

*The text in BOLD indicates the CDR-H3 location along the scFv.

### REFERENCES

1 Wang, C., Horby, P. W., Hayden, F. G. & Gao, G. F. A novel coronavirus outbreak of global health concern. Lancet 395, 470-473, doi: 10.1016/S0140-6736 (20) 30185-9 (2020).
2 Taylor, P. C. et al. Neutralizing monoclonal antibodies for treatment of COVID-19. Nat Rev Immunol 21, 382-393, doi: 10.1038/s41577-021-00542-x (2021).
3 Dong, E., Du, H. & Gardner, L. An interactive web-based dashboard to track COVID-19 in real time. Lancet Infect Dis 20, 533-534, doi: 10.1016/S1473-3099 (20) 30120-1 (2020).
4 Janiaud, P. et al. Association of Convalescent Plasma Treatment With Clinical Outcomes in Patients With COVID-19: A Systematic Review and Meta-analysis. JAMA 325, 1185-1195, doi: 10.1001/jama.2021.2747 (2021).
5 Corti, D. et al. Protective monotherapy against lethal Ebola virus infection by a potently neutralizing antibody. Science 351, 1339-1342, doi: 10.1126/science.aad5224 (2016).
6 Levine, M. M. Monoclonal Antibody Therapy for Ebola Virus Disease. N Engl J Med 381, 2365-2366, doi: 10.1056/NEJMe1915350 (2019).
7 Walker, L. M. et al. Broad and potent neutralizing antibodies from an African donor reveal a new HIV-1 vaccine target. Science 326, 285-289, doi: 10.1126/science. 1178746 (2009).
8 Scheid, J. F. et al. Broad diversity of neutralizing antibodies isolated from memory B cells in HIV-infected individuals. Nature 458, 636-640, doi: 10.1038/nature07930 (2009).
9 Corti, D. et al. Prophylactic and postexposure efficacy of a potent human monoclonal antibody against MERS coronavirus. Proc Natl Acad Sci USA 112, 10473-10478, doi: 10.1073/pnas. 1510199112 (2015).
10 Setthapramote, C. et al. Human monoclonal antibodies to neutralize all dengue virus serotypes using lymphocytes from patients at acute phase of the secondary infection. Biochem Biophys Res Commun 423, 867-872, doi: 10.1016/j.bbrc.2012.06.057 (2012).
11 Kelley, B. Developing therapeutic monoclonal antibodies at pandemic pace. Nat Biotechnol 38, 540-545, doi: 10.1038/s41587-020-0512-5 (2020).
12 Li, M. Applications of display technology in protein analysis. Nat Biotechnol 18, 1251-1256, doi: 10.1038/82355 (2000).
13 Galan, A. et al. Library-based display technologies: where do we stand? Mol Biosyst 12, 2342-2358, doi: 10.1039/c6mb00219f (2016).
14 Hughes-Jones, N. C. et al. Characterization of human blood group scFv antibodies derived from a V gene phage-display library. Br J Haematol 88, 180-186, doi: 10.1111/j.1365-2141.1994.tb04994.x (1994).
15 Dahan, R. et al. TCR-like antibodies distinguish conformational and functional differences in two-versus four-domain auto reactive MHC class II-peptide complexes. Eur J Immunol 41, 1465-1479, doi: 10.1002/eji.201041241 (2011).
16 Griffin, H. M. & Ouwehand, W. H. A human monoclonal antibody specific for the leucine-33 (P1A1, HPA-1a) form of platelet glycoprotein IIIa from a V gene phage display library. Blood 86, 4430-4436 (1995).
17 Rouet, R., Jackson, K. J. L., Langley, D. B. & Christ, D. Next-Generation Sequencing of Antibody Display Repertoires. Front Immunol 9, 118, doi: 10.3389/fimmu.2018.00118 (2018).
18 Miyazaki, N. et al. Isolation and characterization of antigen-specific alpaca (Lama pacos) VHH antibodies by biopanning followed by high-throughput sequencing. J Biochem 158, 205-215, doi: 10.1093/jb/mvv038 (2015).
19 Yang, W. et al. Next-generation sequencing enables the discovery of more diverse positive clones from a phage-displayed antibody library. Exp Mol Med 49, e308, doi: 10.1038/emm.2017.22 (2017).
20 Hwang, M. S. et al. Structural engineering of chimeric antigen receptors targeting HLA-restricted neoantigens. Nat Commun 12, 5271, doi: 10.1038/s41467-021-25605-4 (2021).
21 Hsiue, E. H. et al. Targeting a neoantigen derived from a common TP53 mutation. Science 371, doi: 10.1126/science.abc8697 (2021).
22 Douglass, J. et al. Bispecific antibodies targeting mutant RAS neoantigens. Sci Immunol 6, doi: 10.1126/sciimmunol.abd5515 (2021).
23 Kinde, I., Wu, J., Papadopoulos, N., Kinzler, K. W. & Vogelstein, B. Detection and quantification of rare mutations with massively parallel sequencing. Proc Natl Acad Sci USA 108, 9530-9535, doi: 10.1073/pnas. 1105422108 (2011).
24 Hosseini, S., Vázquez-Villegas, P., Rito-Palomares, M. & Martinez-Chapa, S. O. in Enzyme-linked Immunosorbent Assay (ELISA): From A to Z 67-115 (Springer Singapore, 2018).
25 Hoffmann, M. et al. SARS-COV-2 Cell Entry Depends on ACE2 and TMPRSS2 and Is Blocked by a Clinically Proven Protease Inhibitor. Cell 181, 271-280 e278, doi: 10.1016/j.cell.2020.02.052 (2020).
26 Derda, R. et al. Diversity of phage-displayed libraries of peptides during panning and amplification. Molecules 16, 1776-1803, doi: 10.3390/molecules16021776 (2011).
27 Xiao, X. et al. A high-throughput platform for population reformatting and mammalian expression of phage display libraries to enable functional screening as full-length IgG. MAbs 9, 996-1006, doi: 10.1080/19420862.2017.1337617 (2017).
28 Liu, L. et al. Potent neutralizing antibodies against multiple epitopes on SARS-COV-2 spike. Nature 584, 450-456, doi: 10.1038/s41586-020-2571-7 (2020).
29 Baum, A. et al. Antibody cocktail to SARS-COV-2 spike protein prevents rapid mutational escape seen with individual antibodies. Science 369, 1014-1018, doi: 10.1126/science.abd0831 (2020).
30 Hansen, J. et al. Studies in humanized mice and convalescent humans yield a SARS-COV-2 antibody cocktail. Science 369, 1010-1014, doi: 10.1126/science.abd0827 (2020).
31 Wrapp, D. et al. Cryo-EM structure of the 2019-nCOV spike in the prefusion conformation. Science 367, 1260-1263, doi: 10.1126/science.abb2507 (2020).
32 Miller, M. S. et al. An engineered antibody fragment targeting mutant beta-catenin via major histocompatibility complex I neoantigen presentation. J Biol Chem 294, 19322-19334, doi: 10.1074/jbc.RA119.010251 (2019).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(i) providing a display library comprising a plurality of library members, wherein each library member comprises a display protein and a nucleic acid encoding the display protein, wherein each display protein comprises a complementarity determining region (CDR) H3 region;
(ii) performing a binding assay of the display library against a target antigen to enrich for target-bound display proteins, and isolating nucleic acid from the target bound library members to generate a target bound nucleic acid sample;
(iii) performing a binding assay of the display library against an off-target antigen to enrich for off-target-bound display proteins and isolating nucleic acid from the off-target bound library members to generate an off-target bound nucleic acid sample;
(iv) determining the number of nucleic acid molecules comprising one or more of the CDR H3 regions of the display proteins of the display library in a representative sample of each of the target-bound nucleic acid sample and the off-target bound nucleic acid sample;
(v) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one.

2. The method of claim 1, further comprising:
(vi) comparing the ratio of to a reference value;
(vii) if the ratio is larger than the reference value, determining the sequence of the CDR H1, CDR H2, CDR H3, CDR L1, CDR L2, and CDR L3 sequences of one or more of the display proteins of the display library; and
(viii) optionally, performing a binding assay on the one or more display proteins from step (vii) against the on-target antigen and/or the off-target antigen.

3. A method for identifying an antibody, or antigen binding fragment thereof, that binds to a target antigen, the method comprising:
(i) providing a display library comprising a plurality of library members, wherein each library member comprises a display protein and a nucleic acid encoding the display protein, wherein each display protein comprises a complementarity determining region (CDR) H3 region;
(ii) performing one or more rounds of selection on the display library against a first target antigen to generate a selected display library;
(iii) performing a binding assay of the selected display library against a target antigen to enrich for target-bound display proteins, and isolating nucleic acid from the target bound library members to generate a target bound nucleic acid sample;
(iv) performing a binding assay of the display library against an off-target antigen to enrich for off-target-bound display proteins and isolating nucleic acid from the off-target bound library members to generate an off-target bound nucleic acid sample;
(v) determining the number of nucleic acid molecules comprising one or more of the CDR H3 regions of the display proteins of the display library in a representative sample of each of the target-bound nucleic acid sample and the off-target bound nucleic acid sample; and
(vi) for at least one of the one or more CDR H3 sequences, determining a ratio of the number of molecules comprising the CDR H3 sequence in the target-bound nucleic acid sample to the number of molecules comprising the CDR H3 sequence in the off-target-bound nucleic acid sample, wherein a count of zero in the denominator is rounded to a positive number less than one.

4. The method of claim 3, further comprising:
(vii) comparing the ratio of to a reference value;
(viii) if the ratio is larger than the reference value, determining the sequence of the CDR H1, CDR H2, CDR H3, CDR L1, CDR L2, and CDR L3 sequences of one or more of the display proteins of the display library, thereby identifying an antibody or antigen binding fragment thereof that binds to a target antigen; and
(ix) optionally, performing a binding assay on the one or more display proteins from step (viii) against the on-target antigen and/or the off-target antigen.

5. The method of any one of claims 1-3, wherein the number of library members in the display library is at least 10², at least 10³, at least 10⁴, at least 10⁵, at least 10⁶, at least 10⁷, at least 10⁸, or at least 10⁹.

6. The method of any one of claims 1-5, wherein determining the number of nucleic acid molecules comprises massively parallel sequencing.

7. The method of any one of claims 1-6, wherein massively parallel sequencing comprises sequencing-by-synthesis.

8. The method of any one of claims 1-7, wherein determining the number of nucleic acid molecules comprises PCR amplification using primers that universally amplify the CDR H3 region of the display proteins and also incorporate a molecular barcode.

9. The method of any one of claims 3-8, wherein the one or more rounds of selection comprises one or more rounds comprising positive selection against the target antigen; and/or one or more rounds comprising negative selection against an off-target antigen; and, optionally, amplification.

10. The method of any one of claims 1-9, comprising a first round of selection, wherein the first round of selection comprises or consists of positive selection against the target antigen.

11. The method of claim 10, wherein the first round of selection comprises or consists of positive selection against the target antigen followed by negative selection against the off-target antigen.

12. The method claim 10 or claim 11, wherein the method comprises between two and ten rounds of selection, wherein each of the second through tenth rounds of selection comprises or consists of positive selection against a target antigen and/or negative selection against the off-target antigen to generate a selected display library, and wherein the input display library at the start of each round of selection is the selected display library output from the last round of selection.

13. The method of any one of claims 9-12, wherein positive selection against the target antigen comprises positive selection against a cell line expressing a target antigen and negative selection comprises selection against a cell line not expressing the target antigen.

14. The method of claim 13, wherein the cell line not expressing the target antigen is a genetic knockout or knockdown version of the cell line expressing the target antigen.

15. The method of any one of claims 9-12, wherein positive selection against the target antigen comprises positive selection against a substrate comprising the target antigen and negative selection comprises negative selection against the substrate not comprising the target antigen.

16. The method of any one of claims 1-15, wherein the display library is a phage display library.

17. The method of claim 16, wherein the phage display library is an M13 phage display library.

18. The method of any one of claims 1-17, wherein the display proteins comprise scFvs.

19. The method of claim 18, wherein the display proteins consist of a fusion protein comprising an scFv.

20. The method of claim 19, wherein the fusion protein comprises or consists of a a phage coat protein or portion thereof and an scFv.

21. The method of any one of claims 2-20, wherein the reference value is 1, 2, 3, 4, 5, 6, 7, 8, 9, 10, 11, 12, 13, 14, 15, 16, 17, 18, 19, or 20.

22. An antigen or antigen binding fragment thereof identified by the methods of any one of claims 1-21.


SEQUENCE LISTING (396 sequences)

SEQ ID NO: 1 - DNA, 27 bp, synthetic construct
  source 1..27: mol_type = other DNA
ggataccgctgtctactactgtagccg

SEQ ID NO: 2 - DNA, 23 bp, synthetic construct
  source 1..23: mol_type = other DNA
ctgctcaccgtcaccaatgtgcc

SEQ ID NO: 3 - DNA, 21 bp, synthetic construct
  source 1..21: mol_type = other DNA
actggccgtcgttttacgtcg

SEQ ID NO: 4 - DNA, 32 bp, synthetic construct
  source 1..32: mol_type = other DNA
tgcgctaatggtaaagcgacctttcacgctat

SEQ ID NO: 5 - DNA, 35 bp, synthetic construct
  source 1..35: mol_type = other DNA
ctgcagatgaatagtctgcgtgcagaggatacagc

SEQ ID NO: 6 - protein, 8 aa, synthetic construct
YSDGAFDY

SEQ ID NO: 7 - protein, 9 aa, synthetic construct
FYSSYAFDV

SEQ ID NO: 8 - protein, 11 aa, synthetic construct
NKYYTAYYMDY

SEQ ID NO: 9 - protein, 10 aa, synthetic construct
GYYASYYFDV

SEQ ID NO: 10 - protein, 10 aa, synthetic construct
RYSSTSAFDY

SEQ ID NO: 11 - protein, 8 aa, synthetic construct
GTYFAMDY

SEQ ID NO: 12 - protein, 11 aa, synthetic construct
GFSYYKGYFDY

SEQ ID NO: 13 - protein, 14 aa, synthetic construct
SLSSYLPYTSYFAY

SEQ ID NO: 14 - protein, 9 aa, synthetic construct
ASSAYAFDY

SEQ ID NO: 15 - protein, 10 aa, synthetic construct
SWFSKSAMDY

SEQ ID NO: 16 - protein, 8 aa, synthetic construct
WGSYAFDY

SEQ ID NO: 17 - protein, 9 aa, synthetic construct
ALYSAYFAY

SEQ ID NO: 18 - protein, 10 aa, synthetic construct
GDYYYSYLDY

SEQ ID NO: 19 - protein, 10 aa, synthetic construct
GLSSHSAFDY

SEQ ID NO: 20 - protein, 15 aa, synthetic construct
GFYYYYYSYYMAMDV

SEQ ID NO: 21 - protein, 11 aa, synthetic construct
YMAWSYKYMDY

SEQ ID NO: 22 - protein, 18 aa, synthetic construct
ATSYSHSYSIYYHYYMDY

SEQ ID NO: 23 - protein, 10 aa, synthetic construct
SFSRYGYMDY

SEQ ID NO: 24 - protein, 9 aa, synthetic construct
QMYYEYMDY

SEQ ID NO: 25 - protein, 8 aa, synthetic construct
SYGSYMDY

SEQ ID NO: 26 - protein, 8 aa, synthetic construct
YYGYAMDY

SEQ ID NO: 27 - protein, 10 aa, synthetic construct
SFSSYGYMDY

SEQ ID NO: 28 - protein, 8 aa, synthetic construct
ETMYGFDY

SEQ ID NO: 29 - protein, 10 aa, synthetic construct
GTSSHSAFDY

SEQ ID NO: 30 - protein, 12 aa, synthetic construct
PLYSSFYSAMDY

SEQ ID NO: 31 - protein, 8 aa, synthetic construct
DSYYYMDY

SEQ ID NO: 32 - protein, 9 aa, synthetic construct
GFTWRYFDV

SEQ ID NO: 33 - protein, 9 aa, synthetic construct
GGIPYAFDV

SEQ ID NO: 34 - protein, 10 aa, synthetic construct
GPSSHSAFVY

SEQ ID NO: 35 - protein, 8 aa, synthetic construct
SGSYYFDY

SEQ ID NO: 36 - protein, 8 aa, synthetic construct
SWGVAMDY

SEQ ID NO: 37 - protein, 8 aa, synthetic construct
SYTSYMDY

SEQ ID NO: 38 - protein, 8 aa, synthetic construct
VGYYYFDY

SEQ ID NO: 39 - protein, 9 aa, synthetic construct
WYSRGYMDY

SEQ ID NO: 40 - protein, 10 aa, synthetic construct
QYGYPYYFDY

SEQ ID NO: 41 - protein, 8 aa, synthetic construct
GTYFAMDY

SEQ ID NO: 42 - protein, 8 aa, synthetic construct
ESMYAMDY

SEQ ID NO: 43 - protein, 8 aa, synthetic construct
ETMYGFDY

SEQ ID NO: 44 - protein, 10 aa, synthetic construct
SNYSSYYLDY

SEQ ID NO: 45 - protein, 11 aa, synthetic construct
NKYYTAYYMDY

SEQ ID NO: 46 - protein, 10 aa, synthetic construct
GYYASYYFDV

SEQ ID NO: 47 - protein, 11 aa, synthetic construct
DVKSYRNYFDY

SEQ ID NO: 48 - protein, 8 aa, synthetic construct
SPWYYLDY

SEQ ID NO: 49 - protein, 8 aa, synthetic construct
SLWYYMDY

SEQ ID NO: 50 - protein, 8 aa, synthetic construct
SPWDYMDY

SEQ ID NO: 51 - protein, 10 aa, synthetic construct
SWFSKSAMDY

SEQ ID NO: 52 - protein, 9 aa, synthetic construct
DSVGYAFDY

SEQ ID NO: 53 - protein, 11 aa, synthetic construct
DMYSVRSAMDY

SEQ ID NO: 54 - protein, 10 aa, synthetic construct
SMYQYSSFDY

SEQ ID NO: 55 - protein, 10 aa, synthetic construct
SRYTRYYMDY

SEQ ID NO: 56 - protein, 8 aa, synthetic construct
ESYYGLDY

SEQ ID NO: 57 - protein, 9 aa, synthetic construct
ATSYTYFDV

SEQ ID NO: 58 - protein, 9 aa, synthetic construct
FYSGYGMDY

SEQ ID NO: 59 - protein, 17 aa, synthetic construct
AFSYYYYYSMYYYAVDY

SEQ ID NO: 60 - protein, 8 aa, synthetic construct
AYWYAFDY

SEQ ID NO: 61 - protein, 9 aa, synthetic construct
DNYYFAMDY

SEQ ID NO: 62 - protein, 14 aa, synthetic construct
GFSAIDGYYYYFDV

SEQ ID NO: 63 - protein, 21 aa, synthetic construct
GSWYYSYSYYYYSGYDYYMDV

SEQ ID NO: 64 - protein, 8 aa, synthetic construct
GTYFALDY

SEQ ID NO: 65 - protein, 9 aa, synthetic construct
GYSRYSFDY

SEQ ID NO: 66 - protein, 10 aa, synthetic construct
PHSYQPAFDY

SEQ ID NO: 67 - protein, 8 aa, synthetic construct
SAYYAVDY

SEQ ID NO: 68 - protein, 8 aa, synthetic construct
SWYYAFDY

SEQ ID NO: 69 - protein, 8 aa, synthetic construct
YYAYSFDY

SEQ ID NO: 70 - protein, 8 aa, synthetic construct
YYSSAFDY

SEQ ID NO: 71 - protein, 8 aa, synthetic construct
SPWYYMEY

SEQ ID NO: 72 - protein, 8 aa, synthetic construct
SPCYYMDY

SEQ ID NO: 73 - protein, 8 aa, synthetic construct
SPWYYMDY

SEQ ID NO: 74 - protein, 8 aa, synthetic construct
SPWYYMDY

SEQ ID NO: 75 - protein, 217 aa, synthetic construct
QQKPGKAPKLLIYSASFLYSGVPSRFSGSRSGTDFTLTISSLQPEDFATYYCQQSWTYPVTFGQGTKVEIKRTGGGSGGGGSGGGASEVQLVESGGGLVQPGGSLRLSCAASGFNIYPYRMHWVRQAPGKGLEWVADILPGNGSTSYADSVKGRFTISADTSKNTAYLQMNSLRAEDTAVYYCSRSPWYYMDYWGQGTLVTVSSGGGASDYKDDDDK

SEQ ID NO: 76 - protein, 8 aa, synthetic construct
YSDGAFDY

SEQ ID NO: 77 - protein, 217 aa, synthetic construct
QQKPGKAPKLLIYSASFLYSGVPSRFSGSRSGTDFTLTISSLQPEDFATYYCQQTYRTPITFGQGTKVEIKRTGGGSGGGGSGGGASEVQLVESGGGLVQPGGSLRLSCAASGFNVQGDGMHWVRQAPGKGLEWVAMVGPYSSYTYYADSVKGRFTISADTSKNTAYLQMNSLRAEDTAVYYCSRYSDGAFDYWGQGTLVTVSSGGGASDYKDDDDK

SEQ ID NO: 78 - protein, 9 aa, synthetic construct
FYSSYAFDV

SEQ ID NO: 79 - protein, 218 aa, synthetic construct
QQKPGKAPKLLIYSASFLYSGVPSRFSGSRSGTDFTLTISSLQPEDFATYYCQQSYRSPITFGQGTKVEIKRTGGGSGGGGSGGGASEVQLVESGGGLVQPGGSLRLSCAASGFNITKYSIHWVRQAPGKGLEWVAMVGPYSSYTYYADSVKGRFTISADTSKNTAYLQMNSLRAEDTAVYYCSRFYSSYAFDVWGQGTLVTVSSGGGASDYKDDDDK

SEQ ID NO: 80 - protein, 11 aa, synthetic construct
NKYYTAYYMDY

SEQ ID NO: 81 - protein, 220 aa, synthetic construct
QQKPGKAPKLLIYSASFLYSGVPSRFSGSRSGTDFTLTISSLQPEDFATYYCQQYYVRPITFGQGTKVEIKRTGGGSGGGGSGGGASEVQLVESGGGLVQPGGSLRLSCAASGFNIYSYAMHWVRQAPGKGLEWVASVAPGSGYTYYADSVKGRFTISADTSKNTAYLQMNSLRAEDTAVYYCSRNKYYTAYYMDYWGQGTLVTVSSGGGASDYKDDDDK

SEQ ID NO: 82 - protein, 10 aa, synthetic construct
GYYASYYFDV

SEQ ID NO: 83 - protein, 219 aa, synthetic construct
QQKPGKAPKLLIYSASFLYSGVPSRFSGSRSGTDFTLTISSLQPEDFATYYCQQYHFKPMTFGQGTKVEIKRTGGGSGGGGSGGGASEVQLVESGGGLVQPGGSLRLSCAASGFNIYSYAMHWVRQAPGKGLEWVASFVPGSYATYYADSVKGRFTISADTSKNTAYLQMNSLRAEDTAVYYCSRGYYASYYFDVWGQGTLVTVSSGGGASDYKDDDDK

SEQ ID NO: 84 - protein, 8 aa, synthetic construct
GTYFAMDY

SEQ ID NO: 85 - protein, 217 aa, synthetic construct
QQKPGKAPKLLIYSASFLYSGVPSRFSGSRSGTDFTLTISSLQPEDFATYYCQQFSSYPITFGQGTKVEIKRTGGGSGGGGSGGGASEVQLVESGGGLVQPGGSLRLSCAASGFNLSYYWMHWVRQAPGKGLEWVASIYPDGSYTWYADSVKGRFTISADTSKNTAYLQMNSLRAEDTAVYYCSRGTYFAMDYWGQGTLVTVSSGGGASDYKDDDDK

SEQ ID NO: 86 - protein, 9 aa, synthetic construct
ALYSAYFAY

SEQ ID NO: 87 - protein, 218 aa, synthetic construct
QQKPGKAPKLLIYSASFLYSGVPSRFSGSRSGTDFTLTISSLQPEDFATYYCQQYYRAPPTFGQGTKVEIKRTGGGSGGGGSGGGASEVQLVESGGGLVQPGGSLRLSCAASGFNIFGYYMHWVRQAPGKGLEWVAVVSPGSGYTNYADSVKGRFTISADTSKNTAYLQMNSLRAEDTAVYYCSRALYSAYFAYWGQGTLVTVSSGGGASDYKDDDDK

SEQ ID NO: 88 - protein, 10 aa, synthetic construct
GDYYYSYLDY

SEQ ID NO: 89 - protein, 219 aa, synthetic construct
QQKPGKAPKLLIYSASFLYSGVPSRFSGSRSGTDFTLTISSLQPEDFATYYCQQWETYPATFGQGTKVEIKRTGGGSGGGGSGGGASEVQLVESGGGLVQPGGSLRLSCAASGFNVYYYWMHWVRQAPGKGLEWVASIYPDSDNTYYADSVKGRFTISADTSKNTAYLQMNSLRAEDTAVYYCSRGDYYYSYLDYWGQGTLVTVSSGGGASDYKDDDDK

SEQ ID NO: 90 - protein, 10 aa, synthetic construct
SFSRYGYMDY

SEQ ID NO: 91 - protein, 219 aa, synthetic construct
QQKPGKAPKLLIYSASFLYSGVPSRFSGSRSGTDFTLTISSLQPEDFATYYCQQSWYPPITFGQGTKVEIKRTGGGSGGGGSGGGASEVQLVESGGGLVQPGGSLRLSCAASGFNIYSYYIHWVRQAPGKGLEWVAKIYPDSGYTYYADSVKGRFTISADTSKNTAYLQMNSLRAEDTAVYYCSRSFSRYGYMDYWGQGTLVTVSSGGGASDYKDDDDK

SEQ ID NO: 92 - protein, 10 aa, synthetic construct
SWFSKSAMDY

SEQ ID NO: 93 - protein, 219 aa, synthetic construct
QQKPGKAPKLLIYSASFLYSGVPSRFSGSRSGTDFTLTISSLQPEDFATYYCQQSSHSPITFGQGTKVEIKRTGGGSGGGGSGGGASEVQLVESGGGLVQPGGSLRLSCAASGFNISTWLIHWVRQAPGKGLEWVAVVYPDSSYTYYADSVKGRFTISADTSKNTAYLQMNSLRAEDTAVYYCSRSWFSKSAMDYWGQGTLVTVSSGGGASDYKDDDDK

SEQ ID NO: 94 - protein, 11 aa, synthetic construct
DMYSVRSAMDY

SEQ ID NO: 95 - protein, 220 aa, synthetic construct
QQKPGKAPKLLIYSASFLYSGVPSRFSGSRSGTDFTLTISSLQPEDFATYYCQQYYSYPITFGQGTKVEIKRTGGGSGGGGSGGGASEVQLVESGGGLVQPGGSLRLSCAASGFNVYYSGMHWVRQAPGKGLEWVAKISPDSGSTYYADSVKGRFTISADTSKNTAYLQMNSLRAEDTAVYYCSRDMYSVRSAMDYWGQGTLVTVSSGGGASDYKDDDDK

SEQ ID NO: 96 - protein, 10 aa, synthetic construct
QYGYPYYFDY

SEQ ID NO: 97 - protein, 219 aa, synthetic construct
QQKPGKAPKLLIYSASFLYSGVPSRFSGSRSGTDFTLTISSLQPEDFATYYCQQASSVPVTFGQGTKVEIKRTGGGSGGGGSGGGASEVQLVESGGGLVQPGGSLRLSCAASGFNIYSYGIHWVRQAPGKGLEWVAKVSPSSGYTYYADSVKGRFTISADTSKNTAYLQMNSLRAEDTAVYYCSRQYGYPYYFDYWGQGTLVTVSSGGGASDYKDDDDK

SEQ ID NO: 98 - protein, 11 aa, synthetic construct
DKSTYESAFDY

SEQ ID NO: 99 - protein, 199 aa, synthetic construct
KPGKAPKLLIYSASFLYSGVPSRFSGSRSGTDFTLTISSLQPEDFATYYCQQSFYSPFTFGQGTKVEIKRTGGGSGGGGSGGGASEVQLVESGGGLVQPGGSLRLSCAASGFNISNASMHWVRQAPGKGLEWVAWIYGSGDYTYYADSVKGRFTISADTSKNTAYLQMNSLRAEDTAVYYCSRDKSTYESAFDYWGQGT

SEQ ID NO: 100 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTTTTACTCTCCTTTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTAACGCATCTATGCATTGGGTGCGCCAGGCACCTGGTAAAGGTCTGGAATGGGTTGCCTGGATCTACGGTTCTGGTGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGATAAATCTACCTACGAATCTGCATTTGATTACTGGGGTCAGGGCACC

SEQ ID NO: 101 - protein, 10 aa, synthetic construct
GGVHYGAMDY

SEQ ID NO: 102 - protein, 197 aa, synthetic construct
KPGKAPKLLIYSASFLYSGVPSRFSGSRSGTDFTLTISSLQPEDFATYYCQQYDSPITFGQGTKVEIKRTGGGSGGGGSGGGASEVQLVESGGGLVQPGGSLRLSCAASGFNIDYKFMHWVRQAPGKGLEWVALFGPDQDYTYYADSVKGRFTISADTSKNTAYLQMNSLRAEDTAVYYCSRGGVHYGAMDYWGQGT

SEQ ID NO: 103 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACGATTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGATTACAAATTTATGCATTGGGTGCGCCAGGCACCTGGTAAAGGTCTGGAATGGGTTGCCCTGTTTGGTCCAGATCAGGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTGGTGTTCATTACGGTGCAATGGATTACTGGGGTCAGGGCACC

SEQ ID NO: 104 - protein, 9 aa, synthetic construct
QSSYAYMDY

SEQ ID NO: 105 - protein, 197 aa, synthetic construct
KPGKAPKLLIYSASFLYSGVPSRFSGSRSGTDFTLTISSLQPEDFATYYCQQQWYSPITFGQGTKVEIKRTGGGSGGGGSGGGASEVQLVESGGGLVQPGGSLRLSCAASGFNLADSHIHWVRQAPGKGLEWVAQLYGDSDYTNYADSVKGRFTISADTSKNTAYLQMNSLRAEDTAVYYCSRQSSYAYMDYWGQGT

SEQ ID NO: 106 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACAGTGGTACTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGGCAGATTCTCATATCCATTGGGTGCGCCAGGCACCTGGTAAAGGTCTGGAATGGGTTGCCCAGCTGTACGGTGATTCTGATTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCAGTCTTCTTACGCATACATGGATTACTGGGGTCAGGGCACC

SEQ ID NO: 107 - protein, 9 aa, synthetic construct
NYSSYAFDY

SEQ ID NO: 108 - protein, 197 aa, synthetic construct
KPGKAPKLLIYSASFLYSGVPSRFSGSRSGTDFTLTISSLQPEDFATYYCQQPYSYPVTFGQGTKVEIKRTGGGSGGGGSGGGASEVQLVESGGGLVQPGGSLRLSCAASGFNVYSYQMHWVRQAPGKGLEWVAYVSGYSGNTYYADSVKGRFTISADTSKNTAYLQMNSLRAEDTAVYYCSRNYSSYAFDYWGQGT

SEQ ID NO: 109 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACCATACTCTTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACTCTTACCAGATGCATTGGGTGCGCCAGGCACCTGGTAAAGGTCTGGAATGGGTTGCCTACGTTTCTGGTTACTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACTACTCTTCTTACGCATTTGATTACTGGGGTCAGGGCACC

SEQ ID NO: 110 - protein, 9 aa, synthetic construct
NYGGYAFDY

SEQ ID NO: 111 - protein, 197 aa, synthetic construct
KPGKAPKLLIYSASFLYSGVPSRFSGSRSGTDFTLTISSLQPEDFATYYCQQSSPPPVTFGQGTKVEIKRTGGGSGGGGSGGGASEVQLVESGGGLVQPGGSLRLSCAASGFNLYSYDIHWVRQAPGKGLEWVAQVVPESGNTYYADSVKGRFTISADTSKNTAYLQMNSLRAEDTAVYYCSRNYGGYAFDYWGQGT

SEQ ID NO: 112 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTCTCCACCACCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTACTCTTACGATATCCATTGGGTGCGCCAGGCACCTGGTAAAGGTCTGGAATGGGTTGCCCAGGTTGTTCCAGAATCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACTACGGTGGTTACGCATTTGATTACTGGGGTCAGGGCACC

SEQ ID NO: 113 - protein, 11 aa, synthetic construct
DMYYSSYAVDV

SEQ ID NO: 114 - protein, 198 aa, synthetic construct
KPGKAPKLLIYSASFLYSGVPSRFSGSRSGTDFTLTISSLQPEDFATYYCQQYYEPVTFGQGTKVEIKRTGGGSGGGGSGGGASEVQLVESGGGLVQPGGSLRLSCAASGFNLSSSGMHWVRQAPGKGLEWVALFDPLYDNTMYADSVKGRFTISADTSKNTAYLQMNSLRAEDTAVYYCSRDMYYSSYAVDVWGQGT

SEQ ID NO: 115 - protein, 594 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACGAACCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTTCTTCTGGTATGCATTGGGTGCGCCAGGCACCTGGTAAAGGTCTGGAATGGGTTGCCCTGTTTGATCCACTGTACGATAACACCATGTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGATATGTACTACTCTTCTTACGCAGTTGATGTTTGGGGTCAGGGCACC

SEQ ID NO: 116 - protein, 10 aa, synthetic construct
AEYYSTAFDY

SEQ ID NO: 117 - protein, 198 aa, synthetic construct
KPGKAPKLLIYSASFLYSGVPSRFSGSRSGTDFTLTISSLQPEDFATYYCQQSFVYPHTFGQGTKVEIKRTGGGSGGGGSGGGASEVQLVESGGGLVQPGGSLRLSCAASGFNVYSYSMHWVRQAPGKGLEWVAAINYDGSYTYYADSVKGRFTISADTSKNTAYLQMNSLRAEDTAVYYCSRAEYYSTAFDYWGQGT

SEQ ID NO: 118 - protein, 594 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTTTGTTTACCCTCATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACTCTTACTCTATGCATTGGGTGCGCCAGGCACCTGGTAAAGGTCTGGAATGGGTTGCCGCAATCAACTACGATGGTTCTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGAATACTACTCTACCGCATTTGATTACTGGGGTCAGGGCACC

SEQ ID NO: 119 - protein, 8 aa, synthetic construct
SDSYAFDY

SEQ ID NO: 120 - protein, 196 aa, synthetic construct
KPGKAPKLLIYSASFLYSGVPSRFSGSRSGTDFTLTISSLQPEDFATYYCQQYYWSPITFGQGTKVEIKRTGGGSGGGGSGGGASEVQLVESGGGLVQPGGSLRLSCAASGFNVTYGYMHWVRQAPGKGLEWVAWLLGDYGYTYYADSVKGRFTISADTSKNTAYLQMNSLRAEDTAVYYCSRSDSYAFDYWGQGT

SEQ ID NO: 121 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACTGGTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTACCTACGGTTACATGCATTGGGTGCGCCAGGCACCTGGTAAAGGTCTGGAATGGGTTGCCTGGCTGCTGGGTGATTACGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTGATTCTTACGCATTTGATTACTGGGGTCAGGGCACC

SEQ ID NO: 122 - protein, 8 aa, synthetic construct
YATYAFDY

SEQ ID NO: 123 - protein, 196 aa, synthetic construct
KPGKAPKLLIYSASFLYSGVPSRFSGSRSGTDFTLTISSLQPEDFATYYCQQSFKTPVTFGQGTKVEIKRTGGGSGGGGSGGGASEVQLVESGGGLVQPGGSLRLSCAASGFNISYHGMHWVRQAPGKGLEWVAVIQPSGGYTYYADSVKGRFTISADTSKNTAYLQMNSLRAEDTAVYYCSRYATYAFDYWGQGT

SEQ ID NO: 124 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTTTAAAACCCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTACCATGGTATGCATTGGGTGCGCCAGGCACCTGGTAAAGGTCTGGAATGGGTTGCCGTTATCCAGCCATCTGGTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACGCAACCTACGCATTTGATTACTGGGGTCAGGGCACC

SEQ ID NO: 125 - protein, 10 aa, synthetic construct
GHSYGYALAY

SEQ ID NO: 126 - protein, 198 aa, synthetic construct
KPGKAPKLLIYSASFLYSGVPSRFSGSRSGTDFTLTISSLQPEDFATYYCQQSAYGPITFGQGTKVEIKRTGGGSGGGGSGGGASEVQLVESGGGLVQPGGSLRLSCAASGFNIYHESMHWVRQAPGKGLEWVAEIRPMTGFTYYADSVKGRFTISADTSKNTAYLQMNSLRAEDTAVYYCSRGHSYGYALAYWGQGT

SEQ ID NO: 127 - protein, 594 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTGCATACGGTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACCATGAATCTATGCATTGGGTGCGCCAGGCACCTGGTAAAGGTCTGGAATGGGTTGCCGAAATCAGACCAATGACCGGTTTTACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCATTCTTACGGTTACGCACTGGCATACTGGGGTCAGGGCACC

SEQ ID NO: 128 - protein, 8 aa, synthetic construct
GNGSAFDY

SEQ ID NO: 129 - protein, 196 aa, synthetic construct
KPGKAPKLLIYSASFLYSGVPSRFSGSRSGTDFTLTISSLQPEDFATYYCQQAYGVPITFGQGTKVEIKRTGGGSGGGGSGGGASEVQLVESGGGLVQPGGSLRLSCAASGFNLSYDPMHWVRQAPGKGLEWVAHIEPWSGNTYYADSVKGRFTISADTSKNTAYLQMNSLRAEDTAVYYCSRGNGSAFDYWGQGT

SEQ ID NO: 130 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGCATACGGTGTTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTTACGATCCTATGCATTGGGTGCGCCAGGCACCTGGTAAAGGTCTGGAATGGGTTGCCCATATCGAACCATGGTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTAACGGTTCTGCATTTGATTACTGGGGTCAGGGCACC

SEQ ID NO: 131 - protein, 13 aa, synthetic construct
GLLHHSSGVALDY

SEQ ID NO: 132 - protein, 201 aa, synthetic construct
KPGKAPKLLIYSASFLYSGVPSRFSGSRSGTDFTLTISSLQPEDFATYYCQQSYYYPVTFGQGTKVEIKRTGGGSGGGGSGGGASEVQLVESGGGLVQPGGSLRLSCAASGFNLYNSDMHWVRQAPGKGLEWVAIIYPGSGNTYYADSVKGRFTISADTSKNTAYLQMNSLRAEDTAVYYCSRGLLHHSSGVALDYWGQGT

SEQ ID NO: 133 - protein, 603 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTACTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTACAACTCTGATATGCATTGGGTGCGCCAGGCACCTGGTAAAGGTCTGGAATGGGTTGCCATCATCTACCCAGGTTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCTGCTGCATCATTCTTCTGGTGTTGCACTGGATTACTGGGGTCAGGGCACC

SEQ ID NO: 134 - protein, 10 aa, synthetic construct
WERYSISMDY

SEQ ID NO: 135 - protein, 198 aa, synthetic construct
KPGKAPKLLIYSASFLYSGVPSRFSGSRSGTDFTLTISSLQPEDFATYYCQQSSSYPITFGQGTKVEIKRTGGGSGGGGSGGGASEVQLVESGGGLVQPGGSLRLSCAASGFNIYQYDIHWVRQAPGKGLEWVASLSDSSGSTNYADSVKGRFTISADTSKNTAYLQMNSLRAEDTAVYYCSRWERYSISMDYWGQGT

SEQ ID NO: 136 - protein, 594 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTCTTCTTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACCAGTACGATATCCATTGGGTGCGCCAGGCACCTGGTAAAGGTCTGGAATGGGTTGCCTCTCTGTCTGATTCTTCTGGTTCTACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTGGGAAAGATACTCTATCTCTATGGATTACTGGGGTCAGGGCACC

SEQ ID NO: 137 - protein, 15 aa, synthetic construct
ADWYSLSEFYYSMDY

SEQ ID NO: 138 - protein, 203 aa, synthetic construct
KPGKAPKLLIYSASFLYSGVPSRFSGSRSGTDFTLTISSLQPEDFATYYCQQSSAAPITFGQGTKVEIKRTGGGSGGGGSGGGASEVQLVESGGGLVQPGGSLRLSCAASGFNLGQYDIHWVRQAPGKGLEWVAQFMYRSGYTNYADSVKGRFTISADTSKNTAYLQMNSLRAEDTAVYYCSRADWYSLSEFYYSMDYWGQGT

SEQ ID NO: 139 - protein, 609 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTCTGCAGCACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGGGTCAGTACGATATCCATTGGGTGCGCCAGGCACCTGGTAAAGGTCTGGAATGGGTTGCCCAGTTTATGTACAGATCTGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGATTGGTACTCTCTGTCTGAATTTTACTACTCTATGGATTACTGGGGTCAGGGCACC

SEQ ID NO: 140 - protein, 10 aa, synthetic construct
KMSSAYALDY

SEQ ID NO: 141 - protein, 198 aa, synthetic construct
KPGKAPKLLIYSASFLYSGVPSRFSGSRSGTDFTLTISSLQPEDFATYYCQQSYYLPITFGQGTKVEIKRTGGGSGGGGSGGGASEVQLVESGGGLVQPGGSLRLSCAASGFNFSYEQMHWVRQAPGKGLEWVADFNSGQSNTYYADSVKGRFTISADTSKNTAYLQMNSLRAEDTAVYYCSRKMSSAYALDYWGQGT

SEQ ID NO: 142 - protein, 594 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTACCTGCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTTCTTACGAACAGATGCATTGGGTGCGCCAGGCACCTGGTAAAGGTCTGGAATGGGTTGCCGATTTTAACTCTGGTCAGTCTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAAAATGTCTTCTGCATACGCACTGGATTACTGGGGTCAGGGCACC

SEQ ID NO: 143 - protein, 12 aa, synthetic construct
NDLSSYHYAMDY

SEQ ID NO: 144 - protein, 201 aa, synthetic construct
KPGKAPKLLIYSASFLYSGVPSRFSGSRSGTDFTLTISSLQPEDFATYYCQQGTFSSPLTFGQGTKVEIKRTGGGSGGGGSGGGASEVQLVESGGGLVQPGGSLRLSCAASGFNFTMYSMHWVRQAPGKGLEWVATSWGDSGYTYYADSVKGRFTISADTSKNTAYLQMNSLRAEDTAVYYCSRNDLSSYHYAMDYWGQGT

SEQ ID NO: 145 - protein, 603 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTACCTTTTCTTCTCCTCTGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTACCATGTACTCTATGCATTGGGTGCGCCAGGCACCTGGTAAAGGTCTGGAATGGGTTGCCACCTCTTGGGGTGATTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACGATCTGTCTTCTTACCATTACGCAATGGATTACTGGGGTCAGGGCACC

SEQ ID NO: 146 - protein, 9 aa, synthetic construct
EYQKYSFDY

SEQ ID NO: 147 - protein, 197 aa, synthetic construct
KPGKAPKLLIYSASFLYSGVPSRFSGSRSGTDFTLTISSLQPEDFATYYCQQPYYFPDTFGQGTKVEIKRTGGGSGGGGSGGGASEVQLVESGGGLVQPGGSLRLSCAASGFNIGYSVMHWVRQAPGKGLEWVAVVGPGSGNTNYADSVKGRFTISADTSKNTAYLQMNSLRAEDTAVYYCSREYQKYSFDYWGQGT

SEQ ID NO: 148 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACCATACTACTTTCCTGATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGGTTACTCTGTTATGCATTGGGTGCGCCAGGCACCTGGTAAAGGTCTGGAATGGGTTGCCGTTGTTGGTCCAGGCTCTGGTAACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAATACCAGAAATACTCTTTTGATTACTGGGGTCAGGGCACC

SEQ ID NO: 149 - protein, 19 aa, synthetic construct
AIYNISSASYYTYADGFDY

SEQ ID NO: 150 - protein, 207 aa, synthetic construct
KPGKAPKLLIYSASFLYSGVPSRFSGSRSGTDFTLTISSLQPEDFATYYCQQYSAYPVTFGQGTKVEIKRTGGGSGGGGSGGGASEVQLVESGGGLVQPGGSLRLSCAASGFNVSAIAIHWVRQAPGKGLEWVATIYPGSSYTYYADSVKGRFTISADTSKNTAYLQMNSLRAEDTAVYYCSRAIYNISSASYYTYADGFDYWGQGT

SEQ ID NO: 151 - protein, 621 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTGCATACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTGCAATCGCAATCCATTGGGTGCGCCAGGCACCTGGTAAAGGTCTGGAATGGGTTGCCACCATCTACCCAGGTTCTTCTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAATCTACAACATCTCTTCTGCATCTTACTACACCTACGCAGATGGTTTTGATTACTGGGGTCAGGGCACC

SEQ ID NO: 152 - protein, 9 aa, synthetic construct
ASYSSYFDY

SEQ ID NO: 153 - protein, 196 aa, synthetic construct
KPGKAPKLLIYSASFLYSGVPSRFSGSRSGTDFTLTISSLQPEDFATYYCQQYGRPITFGQGTKVEIKRTGGGSGGGGSGGGASEVQLVESGGGLVQPGGSLRLSCAASGFNIVYQSMHWVRQAPGKGLEWVANLSYDGSYTNYADSVKGRFTISADTSKNTAYLQMNSLRAEDTAVYYCSRASYSSYFDYWGQGT

SEQ ID NO: 154 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACGGTAGACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGTTTACCAGTCTATGCATTGGGTGCGCCAGGCACCTGGTAAAGGTCTGGAATGGGTTGCCAACCTGTCTTACGATGGTTCTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCATCTTACTCTTCTTACTTTGATTACTGGGGTCAGGGCACC

SEQ ID NO: 155 - protein, 8 aa, synthetic construct
WSSSAFDY

SEQ ID NO: 156 - protein, 196 aa, synthetic construct
KPGKAPKLLIYSASFLYSGVPSRFSGSRSGTDFTLTISSLQPEDFATYYCQQGVYGPVTFGQGTKVEIKRTGGGSGGGGSGGGASEVQLVESGGGLVQPGGSLRLSCAASGFNVSYYYMHWVRQAPGKGLEWVAFLNPDSGNTYYADSVKGRFTISADTSKNTAYLQMNSLRAEDTAVYYCSRWSSSAFDYWGQGT

SEQ ID NO: 157 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTGTTTACGGTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTTACTACTACATGCATTGGGTGCGCCAGGCACCTGGTAAAGGTCTGGAATGGGTTGCCTTTCTGAACCCAGATTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTGGTCTTCTTCTGCATTTGATTACTGGGGTCAGGGCACC

SEQ ID NO: 158 - protein, 16 aa, synthetic construct
GRTDYSSYYYSYAFDY

SEQ ID NO: 159 - protein, 204 aa, synthetic construct
KPGKAPKLLIYSASFLYSGVPSRFSGSRSGTDFTLTISSLQPEDFATYYCQQGSSSPSTFGQGTKVEIKRTGGGSGGGGSGGGASEVQLVESGGGLVQPGGSLRLSCAASGFNVSSTYMHWVRQAPGKGLEWVAQIYPHSGPTFYADSVKGRFTISADTSKNTAYLQMNSLRAEDTAVYYCSRGRTDYSSYYYSYAFDYWGQGT

SEQ ID NO: 160 - protein, 612 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTTCTTCTTCTCCTTCTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTTCTACCTACATGCATTGGGTGCGCCAGGCACCTGGTAAAGGTCTGGAATGGGTTGCCCAGATCTACCCACATTCTGGTCCAACCTTTTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTAGAACCGATTACTCTTCTTACTACTACTCTTACGCATTTGATTACTGGGGTCAGGGCACC

SEQ ID NO: 161 - protein, 11 aa, synthetic construct
GNGYYAFAMDY

SEQ ID NO: 162 - protein, 199 aa, synthetic construct
KPGKAPKLLIYSASFLYSGVPSRFSGSRSGTDFTLTISSLQPEDFATYYCQQYSYYPITFGQGTKVEIKRTGGGSGGGGSGGGASEVQLVESGGGLVQPGGSLRLSCAASGFNLSYSYIHWVRQAPGKGLEWVADLYPYGGPTNYADSVKGRFTISADTSKNTAYLQMNSLRAEDTAVYYCSRGNGYYAFAMDYWGQGT

SEQ ID NO: 163 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTACTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTTACTCTTACATCCATTGGGTGCGCCAGGCACCTGGTAAAGGTCTGGAATGGGTTGCCGATCTGTACCCATACGGTGGTCCAACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTAACGGTTACTACGCATTTGCAATGGATTACTGGGGTCAGGGCACC

SEQ ID NO: 164 - protein, 11 aa, synthetic construct
DMYYSSYYMDY

SEQ ID NO: 165 - protein, 199 aa, synthetic construct
KPGKAPKLLIYSASFLYSGVPSRFSGSRSGTDFTLTISSLQPEDFATYYCQQYWYSPITFGQGTKVEIKRTGGGSGGGGSGGGASEVQLVESGGGLVQPGGSLRLSCAASGFNISASGIHWVRQAPGKGLEWVAWIADLSGGTSYADSVKGRFTISADTSKNTAYLQMNSLRAEDTAVYYCSRDMYYSSYYMDYWGQGT

SEQ ID NO: 166 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTGGTACTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGCATCTGGTATCCATTGGGTGCGCCAGGCACCTGGTAAAGGTCTGGAATGGGTTGCCTGGATCGCAGATCTGTCTGGTGGTACCTCTTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGATATGTACTACTCTTCTTACTACATGGATTACTGGGGTCAGGGCACC

SEQ ID NO: 167 - protein, 9 aa, synthetic construct
NYKAYAFDY

SEQ ID NO: 168 - protein, 196 aa, synthetic construct
KPGKAPKLLIYSASFLYSGVPSRFSGSRSGTDFTLTISSLQPEDFATYYCQQSYIPPTFGQGTKVEIKRTGGGSGGGGSGGGASEVQLVESGGGLVQPGGSLRLSCAASGFNIYSYSIHWVRQAPGKGLEWVATIPPDSDATYYADSVKGRFTISADTSKNTAYLQMNSLRAEDTAVYYCSRNYKAYAFDYWGQGT

SEQ ID NO: 169 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACATCCCTCCAACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTCTTACTCTATCCATTGGGTGCGCCAGGCACCTGGTAAAGGTCTGGAATGGGTTGCCACCATCCCACCAGATTCTGATGCAACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACTACAAAGCATACGCATTTGATTACTGGGGTCAGGGCACC

SEQ ID NO: 170 - protein, 8 aa, synthetic construct
YSYDAMDY

SEQ ID NO: 171 - protein, 196 aa, synthetic construct
KPGKAPKLLIYSASFLYSGVPSRFSGSRSGTDFTLTISSLQPEDFATYYCQQISVTPITFGQGTKVEIKRTGGGSGGGGSGGGASEVQLVESGGGLVQPGGSLRLSCAASGFNISHQSMHWVRQAPGKGLEWVAYVYPDSDYTEYADSVKGRFTISADTSKNTAYLQMNSLRAEDTAVYYCSRYSYDAMDYWGQGT

SEQ ID NO: 172 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATCTCTGTTACCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTCATCAGTCTATGCATTGGGTGCGCCAGGCACCTGGTAAAGGTCTGGAATGGGTTGCCTACGTTTACCCAGATTCTGATTACACCGAATATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACTCTTACGATGCAATGGATTACTGGGGTCAGGGCACC

SEQ ID NO: 173 - protein, 9 aa, synthetic construct
YHYMVSFDY

SEQ ID NO: 174 - protein, 197 aa, synthetic construct
KPGKAPKLLIYSASFLYSGVPSRFSGSRSGTDFTLTISSLQPEDFATYYCQQYSSAPITFGQGTKVEIKRTGGGSGGGGSGGGASEVQLVESGGGLVQPGGSLRLSCAASGFNIWSWVMHWVRQAPGKGLEWVALFHPDSGNTYYADSVKGRFTISADTSKNTAYLQMNSLRAEDTAVYYCSRYHYMVSFDYWGQGT

SEQ ID NO: 175 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTCTGCACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTGGTCTTGGGTTATGCATTGGGTGCGCCAGGCACCTGGTAAAGGTCTGGAATGGGTTGCCCTGTTTCATCCAGATTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACCATTACATGGTTTCTTTTGATTACTGGGGTCAGGGCACC

SEQ ID NO: 176 - protein, 9 aa, synthetic construct
SYYQYAFDV

SEQ ID NO: 177 - protein, 197 aa, synthetic construct
KPGKAPKLLIYSASFLYSGVPSRFSGSRSGTDFTLTISSLQPEDFATYYCQQTYTPPRTFGQGTKVEIKRTGGGSGGGGSGGGASEVQLVESGGGLVQPGGSLRLSCAASGFNIYSYSIHWVRQAPGKGLEWVAEFSDQSGYTYYADSVKGRFTISADTSKNTAYLQMNSLRAEDTAVYYCSRSYYQYAFDVWGQGT

SEQ ID NO: 178 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAACCTACACCCCACCTAGAACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTCTTACTCTATCCATTGGGTGCGCCAGGCACCTGGTAAAGGTCTGGAATGGGTTGCCGAATTTTCTGATCAGTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACTACCAGTACGCATTTGATGTTTGGGGTCAGGGCACC

SEQ ID NO: 179 - protein, 9 aa, synthetic construct
GPYYPAFDV

SEQ ID NO: 180 - protein, 197 aa, synthetic construct
KPGKAPKLLIYSASFLYSGVPSRFSGSRSGTDFTLTISSLQPEDFATYYCQQMYSSPITFGQGTKVEIKRTGGGSGGGGSGGGASEVQLVESGGGLVQPGGSLRLSCAASGFNISYYGMHWVRQAPGKGLEWVAEVAPSSGYTYYADSVKGRFTISADTSKNTAYLQMNSLRAEDTAVYYCSRGPYYPAFDVWGQGT

SEQ ID NO: 181 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATGTACTCTTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTACTACGGTATGCATTGGGTGCGCCAGGCACCTGGTAAAGGTCTGGAATGGGTTGCCGAAGTTGCACCATCTTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCCATACTACCCAGCATTTGATGTTTGGGGTCAGGGCACC

SEQ ID NO: 182 - protein, 13 aa, synthetic construct
YSYYSSRNYAMDV

SEQ ID NO: 183 - protein, 201 aa, synthetic construct
KPGKAPKLLIYSASFLYSGVPSRFSGSRSGTDFTLTISSLQPEDFATYYCQQSRSSPVTFGQGTKVEIKRTGGGSGGGGSGGGASEVQLVESGGGLVQPGGSLRLSCAASGFNLFDQYMHWVRQAPGKGLEWVAMLYPGSGQTYYADSVKGRFTISADTSKNTAYLQMNSLRAEDTAVYYCSRYSYYSSRNYAMDVWGQGT

SEQ ID NO: 184 - protein, 603 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTAGATCTTCTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTTTGATCAGTACATGCATTGGGTGCGCCAGGCACCTGGTAAAGGTCTGGAATGGGTTGCCATGCTGTACCCAGGTTCTGGTCAGACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACTCTTACTACTCTTCTAGAAACTACGCAATGGATGTTTGGGGTCAGGGCACC

SEQ ID NO: 185 - protein, 8 aa, synthetic construct
ESGMSMDY

SEQ ID NO: 186 - protein, 195 aa, synthetic construct
KPGKAPKLLIYSASFLYSGVPSRFSGSRSGTDFTLTISSLQPEDFATYYCQQYSYPVTFGQGTKVEIKRTGGGSGGGGSGGGASEVQLVESGGGLVQPGGSLRLSCAASGFNIAGSVMHWVRQAPGKGLEWVAGLNPASGYTIYADSVKGRFTISADTSKNTAYLQMNSLRAEDTAVYYCSRESGMSMDYWGQGT

SEQ ID NO: 187 - protein, 585 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGCAGGTTCTGTTATGCATTGGGTGCGCCAGGCACCTGGTAAAGGTCTGGAATGGGTTGCCGGTCTGAACCCAGCATCTGGTTACACCATCTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAATCTGGTATGTCTATGGATTACTGGGGTCAGGGCACC

SEQ ID NO: 188 - protein, 11 aa, synthetic construct
VVSSEYYAMDY

SEQ ID NO: 189 - protein, 199 aa, synthetic construct
KPGKAPKLLIYSASFLYSGVPSRFSGSRSGTDFTLTISSLQPEDFATYYCQQYKYQPITFGQGTKVEIKRTGGGSGGGGSGGGASEVQLVESGGGLVQPGGSLRLSCAASGFNIRYGAMHWVRQAPGKGLEWVADLYGWSGNTYYADSVKGRFTISADTSKNTAYLQMNSLRAEDTAVYYCSRVVSSEYYAMDYWGQGT

SEQ ID NO: 190 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACAAATACCAGCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCAGATACGGTGCAATGCATTGGGTGCGCCAGGCACCTGGTAAAGGTCTGGAATGGGTTGCCGATCTGTACGGTTGGTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGTTGTTTCTTCTGAATACTACGCAATGGATTACTGGGGTCAGGGCACC

SEQ ID NO: 191 - protein, 7 aa, synthetic construct
YYYAFDY

SEQ ID NO: 192 - protein, 195 aa, synthetic construct
KPGKAPKLLIYSASFLYSGVPSRFSGSRSGTDFTLTISSLQPEDFATYYCQQGSESPITFGQGTKVEIKRTGGGSGGGGSGGGASEVQLVESGGGLVQPGGSLRLSCAASGFNLSYSGMHWVRQAPGKGLEWVAAIYPRSGYTYYADSVKGRFTISADTSKNTAYLQMNSLRAEDTAVYYCSRYYYAFDYWGQGT

SEQ ID NO: 193 - protein, 585 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTTCTGAATCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTTACTCTGGTATGCATTGGGTGCGCCAGGCACCTGGTAAAGGTCTGGAATGGGTTGCCGCAATCTACCCAAGATCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACTACTACGCATTTGATTACTGGGGTCAGGGCACC

SEQ ID NO: 194 - protein, 12 aa, synthetic construct
HDSYHFWYYFDY

SEQ ID NO: 195 - protein, 200 aa, synthetic construct
KPGKAPKLLIYSASFLYSGVPSRFSGSRSGTDFTLTISSLQPEDFATYYCQQYYSYPITFGQGTKVEIKRTGGGSGGGGSGGGASEVQLVESGGGLVQPGGSLRLSCAASGFNIYGSGMHWVRQAPGKGLEWVAVVNPLSGNTYYADSVKGRFTISADTSKNTAYLQMNSLRAEDTAVYYCSRHDSYHFWYYFDYWGQGT

SEQ ID NO: 196 - protein, 600 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACTCTTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACGGTTCTGGTATGCATTGGGTGCGCCAGGCACCTGGTAAAGGTCTGGAATGGGTTGCCGTTGTTAACCCACTGTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATGATTCTTACCATTTTTGGTACTACTTTGATTACTGGGGTCAGGGCACC

SEQ ID NO: 197 - protein, 11 aa, synthetic construct
AMRYYSYYMDY

SEQ ID NO: 198 - protein, 199 aa, synthetic construct
KPGKAPKLLIYSASFLYSGVPSRFSGSRSGTDFTLTISSLQPEDFATYYCQQVYTSPITFGQGTKVEIKRTGGGSGGGGSGGGASEVQLVESGGGLVQPGGSLRLSCAASGFNIYWSWMHWVRQAPGKGLEWVAQIGPGGDYTNYADSVKGRFTISADTSKNTAYLQMNSLRAEDTAVYYCSRAMRYYSYYMDYWGQGT

SEQ ID NO: 199 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGTTTACACCTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTGGTCTTGGATGCATTGGGTGCGCCAGGCACCTGGTAAAGGTCTGGAATGGGTTGCCCAGATCGGTCCAGGTGGTGATTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAATGAGATACTACTCTTACTACATGGATTACTGGGGTCAGGGCACC

SEQ ID NO: 200 - protein, 9 aa, synthetic construct
AHFSSYVDY

SEQ ID NO: 201 - protein, 197 aa, synthetic construct
KPGKAPKLLIYSASFLYSGVPSRFSGSRSGTDFTLTISSLQPEDFATYYCQQYLSLPVTFGQGTKVEIKRTGGGSGGGGSGGGASEVQLVESGGGLVQPGGSLRLSCAASGFNISDQDIHWVRQAPGKGLEWVAMIYPLDSYTWYADSVKGRFTISADTSKNTAYLQMNSLRAEDTAVYYCSRAHFSSYVDYWGQGT

SEQ ID NO: 202 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACCTGTCTCTGCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGATCAGGATATCCATTGGGTGCGCCAGGCACCTGGTAAAGGTCTGGAATGGGTTGCCATGATCTACCCACTGGATTCTTACACCTGGTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCACATTTTTCTTCTTACGTTGATTACTGGGGTCAGGGCACC

SEQ ID NO: 203 - protein, 8 aa, synthetic construct
EYGSAMDY

SEQ ID NO: 204 - protein, 196 aa, synthetic construct
KPGKAPKLLIYSASFLYSGVPSRFSGSRSGTDFTLTISSLQPEDFATYYCQQYRYSPVTFGQGTKVEIKRTGGGSGGGGSGGGASEVQLVESGGGLVQPGGSLRLSCAASGFNIWYWRMHWVRQAPGKGLEWVAELSPRSSYTSYADSVKGRFTISADTSKNTAYLQMNSLRAEDTAVYYCSREYGSAMDYWGQGT

SEQ ID NO: 205 - protein, 588 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACAGATACTCTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTGGTACTGGAGAATGCATTGGGTGCGCCAGGCACCTGGTAAAGGTCTGGAATGGGTTGCCGAACTGTCTCCAAGATCTTCTTACACCTCTTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAATACGGTTCTGCAATGGATTACTGGGGTCAGGGCACC

SEQ ID NO: 206 - protein, 11 aa, synthetic construct
YEAGISEAMDV

SEQ ID NO: 207 - protein, 199 aa, synthetic construct
KPGKAPKLLIYSASFLYSGVPSRFSGSRSGTDFTLTISSLQPEDFATYYCQQGYMDPVTFGQGTKVEIKRTGGGSGGGGSGGGASEVQLVESGGGLVQPGGSLRLSCAASGFNISSYGMHWVRQAPGKGLEWVALLYPQYSYTNYADSVKGRFTISADTSKNTAYLQMNSLRAEDTAVYYCSRYEAGISEAMDVWGQGT

SEQ ID NO: 208 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTTACATGGATCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTTACGGTATGCATTGGGTGCGCCAGGCACCTGGTAAAGGTCTGGAATGGGTTGCCCTGCTGTACCCACAGTACTCTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACGAAGCAGGTATCTCTGAAGCAATGGATGTTTGGGGTCAGGGCACC

SEQ ID NO: 209 - protein, 11 aa, synthetic construct
SLFSYQYAFDY

SEQ ID NO: 210 - protein, 199 aa, synthetic construct
KPGKAPKLLIYSASFLYSGVPSRFSGSRSGTDFTLTISSLQPEDFATYYCQQYFSYPITFGQGTKVEIKRTGGGSGGGGSGGGASEVQLVESGGGLVQPGGSLRLSCAASGFNVFQYTMHWVRQAPGKGLEWVATIYPISGNTYYADSVKGRFTISADTSKNTAYLQMNSLRAEDTAVYYCSRSLFSYQYAFDYWGQGT

SEQ ID NO: 211 - protein, 597 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTTTTCTTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTTTCAGTACACCATGCATTGGGTGCGCCAGGCACCTGGTAAAGGTCTGGAATGGGTTGCCACCATCTACCCAATCTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTCTGTTTTCTTACCAGTACGCATTTGATTACTGGGGTCAGGGCACC

SEQ ID NO: 212 - protein, 9 aa, synthetic construct
SSRSMALDY

SEQ ID NO: 213 - protein, 197 aa, synthetic construct
KPGKAPKLLIYSASFLYSGVPSRFSGSRSGTDFTLTISSLQPEDFATYYCQQYHSYPMTFGQGTKVEIKRTGGGSGGGGSGGGASEVQLVESGGGLVQPGGSLRLSCAASGFNIYNSGMHWVRQAPGKGLEWVAVVWGGSGNTYYADSVKGRFTISADTSKNTAYLQMNSLRAEDTAVYYCSRSSRSMALDYWGQGT

SEQ ID NO: 214 - protein, 591 aa, synthetic construct
AAACCGGGTAAAGCACCGAAACTGCTGATTTATAGCGCCTCTTTCCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACCATTCTTACCCTATGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACAACTCTGGTATGCATTGGGTGCGCCAGGCACCTGGTAAAGGTCTGGAATGGGTTGCCGTTGTTTGGGGTGGTTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTCTAGATCTATGGCACTGGATTACTGGGGTCAGGGCACC

SEQ ID NO: 215 - protein, 8 aa, synthetic construct
WYGPYMDY

SEQ ID NO: 216 - protein, 196 aa, synthetic construct
KPGKAPKLLIYSASFLYSGVPSRFSGSRSGTDFTLTISSLQPEDFATYYCQQGRYDPVTFGQGTKVEIKRTGGGSGGGGSGGGASEVQLVESGGGLVQPGGSLRLSCAASGFNLSYYIMHWVRQAPGKGLEWVAYVYPGYDSTYYADSVKGRFTISADTSKNTAYLQMNSLRAEDTAVYYCSRWYGPYMDYWGQGT

SEQ ID NO: 217 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTAGATACGATCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTTACTACATCATGCATTGGGTGCGCCAGGCACCTGGTAAAGGTCTGGAATGGGTTGCCTACGTTTACCCAGGTTACGATTCTACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTGGTACGGTCCATACATGGATTACTGGGGTCAGGGCACC

SEQ ID NO: 218 - protein, 9 aa, synthetic construct
SSRSLALDY

SEQ ID NO: 219 - protein, 197 aa, synthetic construct
KPGKAPKLLIYSASFLYSGVPSRFSGSRSGTDFTLTISSLQPEDFATYYCQQYHSYPMTFGQGTKVEIKRTGGGSGGGGSGGGASEVQLVESGGGLVQPGGSLRLSCAASGFNIYNSGMHWVRQAPGKGLEWVAVVWGGSGNTYYADSVKGRFTISADTSKNTAYLQMNSLRAEDTAVYYCSRSSRSLALDYWGQGT

SEQ ID NO: 220 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACCATTCTTACCCTATGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACAACTCTGGTATGCATTGGGTGCGCCAGGCACCTGGTAAAGGTCTGGAATGGGTTGCCGTTGTTTGGGGTGGTTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTCTAGATCTCTGGCACTGGATTACTGGGGTCAGGGCACC

SEQ ID NO: 221 - protein, 21 aa, synthetic construct
MDSASSSKTSYHYGSQYSFDY

SEQ ID NO: 222 - protein, 209 aa, synthetic construct
KPGKAPKLLIYSASFLYSGVPSRFSGSRSGTDFTLTISSLQPEDFATYYCQQSTYPPVTFGQGTKVEIKRTGGGSGGGGSGGGASEVQLVESGGGLVQPGGSLRLSCAASGFNISESIMHWVRQAPGKGLEWVAHVYPGYGYTWYADSVKGRFTISADTSKNTAYLQMNSLRAEDTAVYYCSRMDSASSSKTSYHYGSQYSFDYWGQGT

SEQ ID NO: 223 - protein, 627 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTACCTACCCACCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGAATCTATCATGCATTGGGTGCGCCAGGCACCTGGTAAAGGTCTGGAATGGGTTGCCCATGTTTACCCAGGTTACGGTTACACCTGGTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCATGGATTCTGCATCTTCTTCTAAAACCTCTTACCATTACGGTTCTCAGTACTCTTTTGATTACTGGGGTCAGGGCACC

SEQ ID NO: 224 - protein, 9 aa, synthetic construct
GMVSSYMDY

SEQ ID NO: 225 - protein, 197 aa, synthetic construct
KPGKAPKLLIYSASFLYSGVPSRFSGSRSGTDFTLTISSLQPEDFATYYCQQIRYNPMTFGQGTKVEIKRTGGGSGGGGSGGGASEVQLVESGGGLVQPGGSLRLSCAASGFNIYYSEMHWVRQAPGKGLEWVANISPGGGSTYYADSVKGRFTISADTSKNTAYLQMNSLRAEDTAVYYCSRGMVSSYMDYWGQGT

SEQ ID NO: 226 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATCAGATACAACCCTATGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTACTCTGAAATGCATTGGGTGCGCCAGGCACCTGGTAAAGGTCTGGAATGGGTTGCCAACATCTCTCCAGGTGGTGGTTCTACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTATGGTTTCTTCTTACATGGATTACTGGGGTCAGGGCACC

SEQ ID NO: 227 - protein, 8 aa, synthetic construct
SIYSAFDY

SEQ ID NO: 228 - protein, 197 aa, synthetic construct
KPGKAPKLLIYSASFLYSGVPSRFSGSRSGTDFTLTISSLQPEDFATYYCQQDSSSTPFTFGQGTKVEIKRTGGGSGGGGSGGGASEVQLVESGGGLVQPGGSLRLSCAASGFNITGYHMHWVRQAPGKGLEWVAYISGGGYYTYYADSVKGRFTISADTSKNTAYLQMNSLRAEDTAVYYCSRSIYSAFDYWGQGT

SEQ ID NO: 229 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGATTCTTCTTCTACCCCTTTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCACCGGTTACCATATGCATTGGGTGCGCCAGGCACCTGGTAAAGGTCTGGAATGGGTTGCCTACATCTCTGGTGGTGGTTACTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTATCTACTCTGCATTTGATTACTGGGGTCAGGGCACC

SEQ ID NO: 230 - protein, 11 aa, synthetic construct
ASGYAFSALDY

SEQ ID NO: 231 - protein, 199 aa, synthetic construct
KPGKAPKLLIYSASFLYSGVPSRFSGSRSGTDFTLTISSLQPEDFATYYCQQYNYVPYTFGQGTKVEIKRTGGGSGGGGSGGGASEVQLVESGGGLVQPGGSLRLSCAASGFNVYSDSMHWVRQAPGKGLEWVAVIGPDSGYTYYADSVKGRFTISADTSKNTAYLQMNSLRAEDTAVYYCSRASGYAFSALDYWGQGT

SEQ ID NO: 232 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACAACTACGTTCCTTACACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACTCTGATTCTATGCATTGGGTGCGCCAGGCACCTGGTAAAGGTCTGGAATGGGTTGCCGTTATCGGTCCAGATTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCATCTGGTTACGCATTTTCTGCACTGGATTACTGGGGTCAGGGCACC

SEQ ID NO: 233 - protein, 10 aa, synthetic construct
GYYSGYSFDY

SEQ ID NO: 234 - protein, 198 aa, synthetic construct
KPGKAPKLLIYSASFLYSGVPSRFSGSRSGTDFTLTISSLQPEDFATYYCQQYMYEPVTFGQGTKVEIKRTGGGSGGGGSGGGASEVQLVESGGGLVQPGGSLRLSCAASGFNIITYDMHWVRQAPGKGLEWVAVIVDDLGYTNYADSVKGRFTISADTSKNTAYLQMNSLRAEDTAVYYCSRGYYSGYSFDYWGQGT

SEQ ID NO: 235 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACATGTACGAACCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCATCACCTACGATATGCATTGGGTGCGCCAGGCACCTGGTAAAGGTCTGGAATGGGTTGCCGTTATCGTTGATGATCTGGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTTACTACTCTGGTTACTCTTTTGATTACTGGGGTCAGGGCACC

SEQ ID NO: 236 - protein, 9 aa, synthetic construct
AYYYYAFAY

SEQ ID NO: 237 - protein, 197 aa, synthetic construct
KPGKAPKLLIYSASFLYSGVPSRFSGSRSGTDFTLTISSLQPEDFATYYCQQVWLSPITFGQGTKVEIKRTGGGSGGGGSGGGASEVQLVESGGGLVQPGGSLRLSCAASGFNIYDTGIHWVRQAPGKGLEWVATIYDFSSNTYYADSVKGRFTISADTSKNTAYLQMNSLRAEDTAVYYCSRAYYYYAFAYWGQGT

SEQ ID NO: 238 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGTTTGGCTGTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACGATACCGGTATCCATTGGGTGCGCCAGGCACCTGGTAAAGGTCTGGAATGGGTTGCCACCATCTACGATTTTTCTTCTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCATACTACTACTACGCATTTGCATACTGGGGTCAGGGCACC

SEQ ID NO: 239 - protein, 10 aa, synthetic construct
LHIHYMALDV

SEQ ID NO: 240 - protein, 199 aa, synthetic construct
KPGKAPKLLIYSASFLYSGVPSRFSGSRSGTDFTLTISSLQPEDFATYYCQQSLSKPFITFGQGTKVEIKRTGGGSGGGGSGGGASEVQLVESGGGLVQPGGSLRLSCAASGFNISGIGMHWVRQAPGKGLEWVAWVSPKSGYTYYADSVKGRFTISADTSKNTAYLQMNSLRAEDTAVYYCSRLHIHYMALDVWGQGT

SEQ ID NO: 241 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTCTGTCTAAACCTTTTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGGTATCGGTATGCATTGGGTGCGCCAGGCACCTGGTAAAGGTCTGGAATGGGTTGCCTGGGTTTCTCCAAAATCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCTGCATATCCATTACATGGCACTGGATGTTTGGGGTCAGGGCACC

SEQ ID NO: 242 - protein, 8 aa, synthetic construct
SYYSYFDY

SEQ ID NO: 243 - protein, 196 aa, synthetic construct
KPGKAPKLLIYSASFLYSGVPSRFSGSRSGTDFTLTISSLQPEDFATYYCQQSYWLPITFGQGTKVEIKRTGGGSGGGGSGGGASEVQLVESGGGLVQPGGSLRLSCAASGFNVFPGAMHWVRQAPGKGLEWVAMVYDDSSNTYYADSVKGRFTISADTSKNTAYLQMNSLRAEDTAVYYCSRSYYSYFDYWGQGT

SEQ ID NO: 244 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTGGCTGCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTTTCCAGGTGCAATGCATTGGGTGCGCCAGGCACCTGGTAAAGGTCTGGAATGGGTTGCCATGGTTTACGATGATTCTTCTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACTACTCTTACTTTGATTACTGGGGTCAGGGCACC

SEQ ID NO: 245 - protein, 8 aa, synthetic construct
HTPYYMDY

SEQ ID NO: 246 - protein, 196 aa, synthetic construct
KPGKAPKLLIYSASFLYSGVPSRFSGSRSGTDFTLTISSLQPEDFATYYCQQHYNTPITFGQGTKVEIKRTGGGSGGGGSGGGASEVQLVESGGGLVQPGGSLRLSCAASGFNVSRQGIHWVRQAPGKGLEWVAHIWGDSGYTYYADSVKGRFTISADTSKNTAYLQMNSLRAEDTAVYYCSRHTPYYMDYWGQGT

SEQ ID NO: 247 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACATTACAACACCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTAGACAGGGTATCCATTGGGTGCGCCAGGCACCTGGTAAAGGTCTGGAATGGGTTGCCCATATCTGGGGTGATTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATACCCCATACTACATGGATTACTGGGGTCAGGGCACC

SEQ ID NO: 248 - protein, 8 aa, synthetic construct
SPYSSFDY

SEQ ID NO: 249 - protein, 196 aa, synthetic construct
KPGKAPKLLIYSASFLYSGVPSRFSGSRSGTDFTLTISSLQPEDFATYYCQQYVSWPITFGQGTKVEIKRTGGGSGGGGSGGGASEVQLVESGGGLVQPGGSLRLSCAASGFNIEFSGMHWVRQAPGKGLEWVAFIYSDSGYTNYADSVKGRFTISADTSKNTAYLQMNSLRAEDTAVYYCSRSPYSSFDYWGQGT

SEQ ID NO: 250 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACGTTTCTTGGCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGAATTTTCTGGTATGCATTGGGTGCGCCAGGCACCTGGTAAAGGTCTGGAATGGGTTGCCTTTATCTACTCTGATTCTGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTCCATACTCTTCTTTTGATTACTGGGGTCAGGGCACC

SEQ ID NO: 251 - protein, 9 aa, synthetic construct
SYSYSYFDY

SEQ ID NO: 252 - protein, 196 aa, synthetic construct
KPGKAPKLLIYSASFLYSGVPSRFSGSRSGTDFTLTISSLQPEDFATYYCQQAYYDPFTFGQGTKVEIKRTGGGSGGGGSGGGASEVQLVESGGGLVQPGGSLRLSCAASGFNVYYSGMHWVRQAPGKGLEWVAVISGSGYTYYADSVKGRFTISADTSKNTAYLQMNSLRAEDTAVYYCSRSYSYSYFDYWGQGT

SEQ ID NO: 253 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGCATACTACGATCCTTTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACTACTCTGGTATGCATTGGGTGCGCCAGGCACCTGGTAAAGGTCTGGAATGGGTTGCCGTTATCTCTGGTTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACTCTTACTCTTACTTTGATTACTGGGGTCAGGGCACC

SEQ ID NO: 254 - protein, 9 aa, synthetic construct
EYMSQTFDY

SEQ ID NO: 255 - protein, 197 aa, synthetic construct
KPGKAPKLLIYSASFLYSGVPSRFSGSRSGTDFTLTISSLQPEDFATYYCQQNRYVPVTFGQGTKVEIKRTGGGSGGGGSGGGASEVQLVESGGGLVQPGGSLRLSCAASGFNVSGSGIHWVRQAPGKGLEWVAQVSPWSYNTYYADSVKGRFTISADTSKNTAYLQMNSLRAEDTAVYYCSREYMSQTFDYWGQGT

SEQ ID NO: 256 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAAACAGATACGTTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTGGTTCTGGTATCCATTGGGTGCGCCAGGCACCTGGTAAAGGTCTGGAATGGGTTGCCCAGGTTTCTCCATGGTCTTACAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAATACATGTCTCAGACCTTTGATTACTGGGGTCAGGGCACC

SEQ ID NO: 257 - protein, 9 aa, synthetic construct
VWYPYAFDY

SEQ ID NO: 258 - protein, 197 aa, synthetic construct
KPGKAPKLLIYSASFLYSGVPSRFSGSRSGTDFTLTISSLQPEDFATYYCQQYSSSPITFGQGTKVEIKRTGGGSGGGGSGGGASEVQLVESGGGLVQPGGSLRLSCAASGFNISEYDIHWVRQAPGKGLEWVADVDDQMGYTYYADSVKGRFTISADTSKNTAYLQMNSLRAEDTAVYYCSRVWYPYAFDYWGQGT

SEQ ID NO: 259 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTCTTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGAATACGATATCCATTGGGTGCGCCAGGCACCTGGTAAAGGTCTGGAATGGGTTGCCGATGTTGATGATCAGATGGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGTTTGGTACCCATACGCATTTGATTACTGGGGTCAGGGCACC

SEQ ID NO: 260 - protein, 8 aa, synthetic construct
HDYYYLDY

SEQ ID NO: 261 - protein, 196 aa, synthetic construct
KPGKAPKLLIYSASFLYSGVPSRFSGSRSGTDFTLTISSLQPEDFATYYCQQLYSTPVTFGQGTKVEIKRTGGGSGGGGSGGGASEVQLVESGGGLVQPGGSLRLSCAASGFNIGYYGIHWVRQAPGKGLEWVAHISPGPGNTYYADSVKGRFTISADTSKNTAYLQMNSLRAEDTAVYYCSRHDYYYLDYWGQGT

SEQ ID NO: 262 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACTGTACTCTACCCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGGTTACTACGGTATCCATTGGGTGCGCCAGGCACCTGGTAAAGGTCTGGAATGGGTTGCCCATATCTCTCCAGGTCCAGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATGATTACTACTACCTGGATTACTGGGGTCAGGGCACC

SEQ ID NO: 263 - protein, 9 aa, synthetic construct
GNPYGAFDY

SEQ ID NO: 264 - protein, 197 aa, synthetic construct
KPGKAPKLLIYSASFLYSGVPSRFSGSRSGTDFTLTISSLQPEDFATYYCQQSRYYPITFGQGTKVEIKRTGGGSGGGGSGGGASEVQLVESGGGLVQPGGSLRLSCAASGFNIAYYSMHWVRQAPGKGLEWVAAVYPYSDYTYYADSVKGRFTISADTSKNTAYLQMNSLRAEDTAVYYCSRGNPYGAFDYWGQGT

SEQ ID NO: 265 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTAGATACTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCGCATACTACTCTATGCATTGGGTGCGCCAGGCACCTGGTAAAGGTCTGGAATGGGTTGCCGCAGTTTACCCATACTCTGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTAACCCATACGGTGCATTTGATTACTGGGGTCAGGGCACC

SEQ ID NO: 266 - protein, 11 aa, synthetic construct
AHDYSYRAFDH

SEQ ID NO: 267 - protein, 199 aa, synthetic construct
KPGKAPKLLIYSASFLYSGVPSRFSGSRSGTDFTLTISSLQPEDFATYYCQQIREFPHTFGQGTKVEIKRTGGGSGGGGSGGGASEVQLVESGGGLVQPGGSLRLSCAASGFNITYYDMHWVRQAPGKGLEWVAAIIDDSGDTYYADSVKGRFTISADTSKNTAYLQMNSLRAEDTAVYYCSRAHDYSYRAFDHWGQGT

SEQ ID NO: 268 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATCAGAGAATTTCCTCATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCACCTACTACGATATGCATTGGGTGCGCCAGGCACCTGGTAAAGGTCTGGAATGGGTTGCCGCAATCATCGATGATTCTGGTGATACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCACATGATTACTCTTACAGAGCATTTGATCACTGGGGTCAGGGCACC

SEQ ID NO: 269 - protein, 9 aa, synthetic construct
YEPGYALDY

SEQ ID NO: 270 - protein, 197 aa, synthetic construct
KPGKAPKLLIYSASFLYSGVPSRFSGSRSGTDFTLTISSLQPEDFATYYCQQSYYYPFTFGQGTKVEIKRTGGGSGGGGSGGGASEVQLVESGGGLVQPGGSLRLSCAASGFNIRHYDMHWVRQAPGKGLEWVAHILPYPGYTYYADSVKGRFTISADTSKNTAYLQMNSLRAEDTAVYYCSRYEPGYALDYWGQGT

SEQ ID NO: 271 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTACTACCCTTTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCAGACATTACGATATGCATTGGGTGCGCCAGGCACCTGGTAAAGGTCTGGAATGGGTTGCCCATATCCTGCCATACCCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACGAACCAGGTTACGCACTGGATTACTGGGGTCAGGGCACC

SEQ ID NO: 272 - protein, 8 aa, synthetic construct
YSYDAIDY

SEQ ID NO: 273 - protein, 196 aa, synthetic construct
KPGKAPKLLIYSASFLYSGVPSRFSGSRSGTDFTLTISSLQPEDFATYYCQQISVTPITFGQGTKVEIKRTGGGSGGGGSGGGASEVQLVESGGGLVQPGGSLRLSCAASGFNISHQSMHWVRQAPGKGLEWVAYVYPDSDYTEYADSVKGRFTISADTSKNTAYLQMNSLRAEDTAVYYCSRYSYDAIDYWGQGT

SEQ ID NO: 274 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATCTCTGTTACCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTCATCAGTCTATGCATTGGGTGCGCCAGGCACCTGGTAAAGGTCTGGAATGGGTTGCCTACGTTTACCCAGATTCTGATTACACCGAATATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACTCTTACGATGCAATTGATTACTGGGGTCAGGGCACC

SEQ ID NO: 275 - protein, 11 aa, synthetic construct
ATSYGYSAMDY

SEQ ID NO: 276 - protein, 199 aa, synthetic construct
KPGKAPKLLIYSASFLYSGVPSRFSGSRSGTDFTLTISSLQPEDFATYYCQQYYSSPWTFGQGTKVEIKRTGGGSGGGGSGGGASEVQLVESGGGLVQPGGSLRLSCAASGFNISSPVMHWVRQAPGKGLEWVATLDPDYSYTNYADSVKGRFTISADTSKNTAYLQMNSLRAEDTAVYYCSRATSYGYSAMDYWGQGT

SEQ ID NO: 277 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACTCTTCTCCTTGGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTCCAGTTATGCATTGGGTGCGCCAGGCACCTGGTAAAGGTCTGGAATGGGTTGCCACCCTGGATCCAGATTACTCTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAACCTCTTACGGTTACTCTGCAATGGATTACTGGGGTCAGGGCACC

SEQ ID NO: 278 - protein, 10 aa, synthetic construct
SIYWDAAMDY

SEQ ID NO: 279 - protein, 198 aa, synthetic construct
KPGKAPKLLIYSASFLYSGVPSRFSGSRSGTDFTLTISSLQPEDFATYYCQQSSNYPHTFGQGTKVEIKRTGGGSGGGGSGGGASEVQLVESGGGLVQPGGSLRLSCAASGFNVSSTYMHWVRQAPGKGLEWVAMFYYYRGYTYYADSVKGRFTISADTSKNTAYLQMNSLRAEDTAVYYCSRSIYWDAAMDYWGQGT

SEQ ID NO: 280 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTCTAACTACCCTCATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTTCTACCTACATGCATTGGGTGCGCCAGGCACCTGGTAAAGGTCTGGAATGGGTTGCCATGTTTTACTACTACAGAGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTATCTACTGGGATGCAGCAATGGATTACTGGGGTCAGGGCACC

SEQ ID NO: 281 - protein, 12 aa, synthetic construct
GSYGHYGYSMDY

SEQ ID NO: 282 - protein, 200 aa, synthetic construct
KPGKAPKLLIYSASFLYSGVPSRFSGSRSGTDFTLTISSLQPEDFATYYCQQYSIEPDTFGQGTKVEIKRTGGGSGGGGSGGGASEVQLVESGGGLVQPGGSLRLSCAASGFNISSSIIHWVRQAPGKGLEWVAMFPPYSGNTYYADSVKGRFTISADTSKNTAYLQMNSLRAEDTAVYYCSRGSYGHYGYSMDYWGQGT

SEQ ID NO: 283 - protein, 600 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTATCGAACCTGATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTTCTATCATCCATTGGGTGCGCCAGGCACCTGGTAAAGGTCTGGAATGGGTTGCCATGTTTCCACCATACTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTTCTTACGGTCATTACGGTTACTCTATGGATTACTGGGGTCAGGGCACC

SEQ ID NO: 284 - protein, 11 aa, synthetic construct
SGSYAFSAMDV

SEQ ID NO: 285 - protein, 199 aa, synthetic construct
KPGKAPKLLIYSASFLYSGVPSRFSGSRSGTDFTLTISSLQPEDFATYYCQQYSYMPVTFGQGTKVEIKRTGGGSGGGGSGGGASEVQLVESGGGLVQPGGSLRLSCAASGFNVAGSDIHWVRQAPGKGLEWVAFVYPGYGVTNYADSVKGRFTISADTSKNTAYLQMNSLRAEDTAVYYCSRSGSYAFSAMDVWGQGT

SEQ ID NO: 286 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTACATGCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTGCAGGTTCTGATATCCATTGGGTGCGCCAGGCACCTGGTAAAGGTCTGGAATGGGTTGCCTTTGTTTACCCAGGTTACGGTGTTACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTGGTTCTTACGCATTTTCTGCAATGGATGTTTGGGGTCAGGGCACC

SEQ ID NO: 287 - protein, 10 aa, synthetic construct
ADEYWGAVDY

SEQ ID NO: 288 - protein, 198 aa, synthetic construct
KPGKAPKLLIYSASFLYSGVPSRFSGSRSGTDFTLTISSLQPEDFATYYCQQTLIYPITFGQGTKVEIKRTGGGSGGGGSGGGASEVQLVESGGGLVQPGGSLRLSCAASGFNISSYMIHWVRQAPGKGLEWVAVIYGSSDYTYYADSVKGRFTISADTSKNTAYLQMNSLRAEDTAVYYCSRADEYWGAVDYWGQGT

SEQ ID NO: 289 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAACCCTGATCTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTTACATGATCCATTGGGTGCGCCAGGCACCTGGTAAAGGTCTGGAATGGGTTGCCGTTATCTACGGTTCTTCTGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGATGAATACTGGGGTGCAGTTGATTACTGGGGTCAGGGCACC

SEQ ID NO: 290 - protein, 9 aa, synthetic construct
GQTESAVDV

SEQ ID NO: 291 - protein, 197 aa, synthetic construct
KPGKAPKLLIYSASFLYSGVPSRFSGSRSGTDFTLTISSLQPEDFATYYCQQRYTIPITFGQGTKVEIKRTGGGSGGGGSGGGASEVQLVESGGGLVQPGGSLRLSCAASGFNLGYYMMHWVRQAPGKGLEWVAMFYGDSGYTNYADSVKGRFTISADTSKNTAYLQMNSLRAEDTAVYYCSRGQTESAVDVWGQGT

SEQ ID NO: 292 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAAGATACACCATCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGGGTTACTACATGATGCATTGGGTGCGCCAGGCACCTGGTAAAGGTCTGGAATGGGTTGCCATGTTTTACGGTGATTCTGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCAGACCGAATCTGCAGTTGATGTTTGGGGTCAGGGCACC

SEQ ID NO: 293 - protein, 11 aa, synthetic construct
SSYYAYSAMDY

SEQ ID NO: 294 - protein, 199 aa, synthetic construct
KPGKAPKLLIYSASFLYSGVPSRFSGSRSGTDFTLTISSLQPEDFATYYCQQYYYHPITFGQGTKVEIKRTGGGSGGGGSGGGASEVQLVESGGGLVQPGGSLRLSCAASGFNVFRSGMHWVRQAPGKGLEWVASLQDDSGYTYYADSVKGRFTISADTSKNTAYLQMNSLRAEDTAVYYCSRSSYYAYSAMDYWGQGT

SEQ ID NO: 295 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACTACCATCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTTTAGATCTGGTATGCATTGGGTGCGCCAGGCACCTGGTAAAGGTCTGGAATGGGTTGCCTCTCTGCAGGATGATTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTCTTACTACGCATACTCTGCAATGGATTACTGGGGTCAGGGCACC

SEQ ID NO: 296 - protein, 9 aa, synthetic construct
ASYIYAFDY

SEQ ID NO: 297 - protein, 197 aa, synthetic construct
KPGKAPKLLIYSASFLYSGVPSRFSGSRSGTDFTLTISSLQPEDFATYYCQQNYYYPVTFGQGTKVEIKRTGGGSGGGGSGGGASEVQLVESGGGLVQPGGSLRLSCAASGFNIMYEQMHWVRQAPGKGLEWVAIIAPLDDYTNYADSVKGRFTISADTSKNTAYLQMNSLRAEDTAVYYCSRASYIYAFDYWGQGT

SEQ ID NO: 298 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAAACTACTACTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCATGTACGAACAGATGCATTGGGTGCGCCAGGCACCTGGTAAAGGTCTGGAATGGGTTGCCATCATCGCACCACTGGATGATTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCATCTTACATCTACGCATTTGATTACTGGGGTCAGGGCACC

SEQ ID NO: 299 - protein, 8 aa, synthetic construct
GTGYAFDY

SEQ ID NO: 300 - protein, 196 aa, synthetic construct
KPGKAPKLLIYSASFLYSGVPSRFSGSRSGTDFTLTISSLQPEDFATYYCQQHYYVPLTFGQGTKVEIKRTGGGSGGGGSGGGASEVQLVESGGGLVQPGGSLRLSCAASGFNIHYNAMHWVRQAPGKGLEWVAFVSPDGYYTNYADSVKGRFTISADTSKNTAYLQMNSLRAEDTAVYYCSRGTGYAFDYWGQGT

SEQ ID NO: 301 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACATTACTACGTTCCTCTGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCCATTACAACGCAATGCATTGGGTGCGCCAGGCACCTGGTAAAGGTCTGGAATGGGTTGCCTTTGTTTCTCCAGATGGTTACTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTACCGGTTACGCATTTGATTACTGGGGTCAGGGCACC

SEQ ID NO: 302 - protein, 11 aa, synthetic construct
ARYSTQYAFDY

SEQ ID NO: 303 - protein, 199 aa, synthetic construct
KPGKAPKLLIYSASFLYSGVPSRFSGSRSGTDFTLTISSLQPEDFATYYCQQSYSSPLTFGQGTKVEIKRTGGGSGGGGSGGGASEVQLVESGGGLVQPGGSLRLSCAASGFNIMPEGIHWVRQAPGKGLEWVAWIWYDSDYTNYADSVKGRFTISADTSKNTAYLQMNSLRAEDTAVYYCSRARYSTQYAFDYWGQGT

SEQ ID NO: 304 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTCTTCTCCTCTGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCATGCCAGAAGGTATCCATTGGGTGCGCCAGGCACCTGGTAAAGGTCTGGAATGGGTTGCCTGGATCTGGTACGATTCTGATTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAAGATACTCTACCCAGTACGCATTTGATTACTGGGGTCAGGGCACC

SEQ ID NO: 305 - protein, 13 aa, synthetic construct
DWASTSGYHYLDY

SEQ ID NO: 306 - protein, 201 aa, synthetic construct
KPGKAPKLLIYSASFLYSGVPSRFSGSRSGTDFTLTISSLQPEDFATYYCQQGDSYPHTFGQGTKVEIKRTGGGSGGGGSGGGASEVQLVESGGGLVQPGGSLRLSCAASGFNISWTGMHWVRQAPGKGLEWVAWISDGSSWTYYADSVKGRFTISADTSKNTAYLQMNSLRAEDTAVYYCSRDWASTSGYHYLDYWGQGT

SEQ ID NO: 307 - protein, 603 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTGATTCTTACCCTCATACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTGGACCGGTATGCATTGGGTGCGCCAGGCACCTGGTAAAGGTCTGGAATGGGTTGCCTGGATCTCTGATGGTTCTTCTTGGACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGATTGGGCATCTACCTCTGGTTACCATTACCTGGATTACTGGGGTCAGGGCACC

SEQ ID NO: 308 - protein, 9 aa, synthetic construct
YEPGYALDY

SEQ ID NO: 309 - protein, 197 aa, synthetic construct; identical to SEQ ID NO: 270

SEQ ID NO: 310 - protein, 591 aa, synthetic construct; identical to SEQ ID NO: 271

SEQ ID NO: 311 - protein, 11 aa, synthetic construct
SKSYFEGAMDY

SEQ ID NO: 312 - protein, 199 aa, synthetic construct
KPGKAPKLLIYSASFLYSGVPSRFSGSRSGTDFTLTISSLQPEDFATYYCQQGYYSPLTFGQGTKVEIKRTGGGSGGGGSGGGASEVQLVESGGGLVQPGGSLRLSCAASGFNVSKSAMHWVRQAPGKGLEWVAWIYPDGGYTNYADSVKGRFTISADTSKNTAYLQMNSLRAEDTAVYYCSRSKSYFEGAMDYWGQGT

SEQ ID NO: 313 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTTACTACTCTCCTCTGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTAAATCTGCAATGCATTGGGTGCGCCAGGCACCTGGTAAAGGTCTGGAATGGGTTGCCTGGATCTACCCAGATGGTGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTAAATCTTACTTTGAAGGTGCAATGGATTACTGGGGTCAGGGCACC

SEQ ID NO: 314 - protein, 10 aa, synthetic construct
DMLYEDGFDV

SEQ ID NO: 315 - protein, 198 aa, synthetic construct
KPGKAPKLLIYSASFLYSGVPSRFSGSRSGTDFTLTISSLQPEDFATYYCQQAGYSPITFGQGTKVEIKRTGGGSGGGGSGGGASEVQLVESGGGLVQPGGSLRLSCAASGFNFYQYGMHWVRQAPGKGLEWVAFIWPGSSYTYYADSVKGRFTISADTSKNTAYLQMNSLRAEDTAVYYCSRDMLYEDGFDVWGQGT

SEQ ID NO: 316 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGCAGGTTACTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTTACCAGTACGGTATGCATTGGGTGCGCCAGGCACCTGGTAAAGGTCTGGAATGGGTTGCCTTTATCTGGCCAGGTTCTTCTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGATATGCTGTACGAAGATGGTTTTGATGTTTGGGGTCAGGGCACC

SEQ ID NO: 317 - protein, 8 aa, synthetic construct
HMGDAMDY

SEQ ID NO: 318 - protein, 196 aa, synthetic construct
KPGKAPKLLIYSASFLYSGVPSRFSGSRSGTDFTLTISSLQPEDFATYYCQQNKSVPVTFGQGTKVEIKRTGGGSGGGGSGGGASEVQLVESGGGLVQPGGSLRLSCAASGFNLSGYGMHWVRQAPGKGLEWVAHILPWSGNTYYADSVKGRFTISADTSKNTAYLQMNSLRAEDTAVYYCSRHMGDAMDYWGQGT

SEQ ID NO: 319 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAAACAAATCTGTTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTGGTTACGGTATGCATTGGGTGCGCCAGGCACCTGGTAAAGGTCTGGAATGGGTTGCCCATATCCTGCCATGGTCTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATATGGGTGATGCAATGGATTACTGGGGTCAGGGCACC

SEQ ID NO: 320 - protein, 11 aa, synthetic construct
YHWSNYYAFDV

SEQ ID NO: 321 - protein, 199 aa, synthetic construct
KPGKAPKLLIYSASFLYSGVPSRFSGSRSGTDFTLTISSLQPEDFATYYCQQLSAPPITFGQGTKVEIKRTGGGSGGGGSGGGASEVQLVESGGGLVQPGGSLRLSCAASGFNVYTYDMHWVRQAPGKGLEWVADVYPWSYYTNYADSVKGRFTISADTSKNTAYLQMNSLRAEDTAVYYCSRYHWSNYYAFDVWGQGT

SEQ ID NO: 322 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACTGTCTGCACCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACACCTACGATATGCATTGGGTGCGCCAGGCACCTGGTAAAGGTCTGGAATGGGTTGCCGATGTTTACCCATGGTCTTACTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACCATTGGTCTAACTACTACGCATTTGATGTTTGGGGTCAGGGCACC

SEQ ID NO: 323 - protein, 11 aa, synthetic construct
EMAHDSYAMDV

SEQ ID NO: 324 - protein, 199 aa, synthetic construct
KPGKAPKLLIYSAYFLYSGVPSRFSGSRSGTDFTLTISSLQPEDFATYYCQQYTSSPITFGQGTKVEIKRTGGGSGGGGSGGGASEVQLVESGGGLVQPGGSLRLSCAASGFNFSRQIMHWVRQAPGKGLEWVAWIEGSSGNTNYADSVKGRFTISADTSKNTAYLQMNSLRAEDTAVYYCSREMAHDSYAMDVWGQGT

SEQ ID NO: 325 - protein, 597 aa, synthetic construct
AAACCGGGTAAAGCACCGAAACTGCTGATTTATAGCGCCTACTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACACCTCTTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTTCTAGACAGATCATGCATTGGGTGCGCCAGGCACCTGGTAAAGGTCTGGAATGGGTTGCCTGGATCGAAGGTTCTTCTGGTAACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAAATGGCACATGATTCTTACGCAATGGATGTTTGGGGTCAGGGCACC

SEQ ID NO: 326 - protein, 9 aa, synthetic construct
QYYQEAFDY

SEQ ID NO: 327 - protein, 198 aa, synthetic construct
KPGKAPKLLIYSASFLYSGVPSRFSGSRSGTDFTLTISSLQPEDFATYYCQQYSYYGPITFGQGTKVEIKRTGGGSGGGGSGGGASEVQLVESGGGLVQPGGSLRLSCAASGFNISSQYIHWVRQAPGKGLEWVATLYPSSGNTPYADSVKGRFTISADTSKNTAYLQMNSLRAEDTAVYYCSRQYYQEAFDYWGQGT

SEQ ID NO: 328 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTACTACGGTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTCAGTACATCCATTGGGTGCGCCAGGCACCTGGTAAAGGTCTGGAATGGGTTGCCACCCTGTACCCATCTTCTGGTAACACCCCATATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCAGTACTACCAGGAAGCATTTGATTACTGGGGTCAGGGCACC

SEQ ID NO: 329 - protein, 8 aa, synthetic construct
VASTAFDY

SEQ ID NO: 330 - protein, 196 aa, synthetic construct
KPGKAPKLLIYSASFLYSGVPSRFSGSRSGTDFTLTISSLQPEDFATYYCQQTRWYPVTFGQGTKVEIKRTGGGSGGGGSGGGASEVQLVESGGGLVQPGGSLRLSCAASGFNFSSIPMHWVRQAPGKGLEWVADIYGSSGHTNYADSVKGRFTISADTSKNTAYLQMNSLRAEDTAVYYCSRVASTAFDYWGQGT

SEQ ID NO: 331 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAACCAGATGGTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTTCTTCTATCCCAATGCATTGGGTGCGCCAGGCACCTGGTAAAGGTCTGGAATGGGTTGCCGATATCTACGGTTCTTCTGGTCATACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGTTGCATCTACCGCATTTGATTACTGGGGTCAGGGCACC

SEQ ID NO: 332 - protein, 11 aa, synthetic construct
ANLGYISYMDY

SEQ ID NO: 333 - protein, 199 aa, synthetic construct
KPGKAPKLLIYSASFLYSGVPSRFSGSRSGTDFTLTISSLQPEDFATYYCQQGYFEPYTFGQGTKVEIKRTGGGSGGGGSGGGASEVQLVESGGGLVQPGGSLRLSCAASGFNVSKTGMHWVRQAPGKGLEWVALIWPSMGYTSYADSVKGRFTISADTSKNTAYLQMNSLRAEDTAVYYCSRANLGYISYMDYWGQGT

SEQ ID NO: 334 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGGTTACTTTGAACCTTACACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTAAAACCGGTATGCATTGGGTGCGCCAGGCACCTGGTAAAGGTCTGGAATGGGTTGCCCTGATCTGGCCATCTATGGGTTACACCTCTTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAAACCTGGGTTACATCTCTTACATGGATTACTGGGGTCAGGGCACC

SEQ ID NO: 335 - protein, 16 aa, synthetic construct
SYQSPSGSYYYHAFDY

SEQ ID NO: 336 - protein, 204 aa, synthetic construct
KPGKAPKLLIYSASFLYSGVPSRFSGSRSGTDFTLTISSLQPEDFATYYCQQSTTAPITFGQGTKVEIKRTGGGSGGGGSGGGASEVQLVESGGGLVQPGGSLRLSCAASGFNVSYYGMHWVRQAPGKGLEWVAAVVGDSDYTYYADSVKGRFTISADTSKNTAYLQMNSLRAEDTAVYYCSRSYQSPSGSYYYHAFDYWGQGT

SEQ ID NO: 337 - protein, 612 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTACCACCGCACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTTACTACGGTATGCATTGGGTGCGCCAGGCACCTGGTAAAGGTCTGGAATGGGTTGCCGCAGTTGTTGGTGATTCTGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACCAGTCTCCATCTGGTTCTTACTACTACCATGCATTTGATTACTGGGGTCAGGGCACC

SEQ ID NO: 338 - protein, 10 aa, synthetic construct
SDGSSYALDY

SEQ ID NO: 339 - protein, 198 aa, synthetic construct
KPGKAPKLLIYSASFLYSGVPSRFSGSRSGTDFTLTISSLQPEDFATYYCQQKYYRPVTFGQGTKVEIKRTGGGSGGGGSGGGASEVQLVESGGGLVQPGGSLRLSCAASGFNIYNQDMHWVRQAPGKGLEWVAELWGYSDHTNYADSVKGRFTISADTSKNTAYLQMNSLRAEDTAVYYCSRSDGSSYALDYWGQGT

SEQ ID NO: 340 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAAAATACTACAGACCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACAACCAGGATATGCATTGGGTGCGCCAGGCACCTGGTAAAGGTCTGGAATGGGTTGCCGAACTGTGGGGTTACTCTGATCATACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTGATGGTTCTTCTTACGCACTGGATTACTGGGGTCAGGGCACC

SEQ ID NO: 341 - protein, 9 aa, synthetic construct
SYDVYAFDY

SEQ ID NO: 342 - protein, 197 aa, synthetic construct
KPGKAPKLLIYSASFLYSGVPSRFSGSRSGTDFTLTISSLQPEDFATYYCQQSWEMPVTFGQGTKVEIKRTGGGSGGGGSGGGASEVQLVESGGGLVQPGGSLRLSCAASGFNVYAYQMHWVRQAPGKGLEWVAFISDDSGNTNYADSVKGRFTISADTSKNTAYLQMNSLRAEDTAVYYCSRSYDVYAFDYWGQGT

SEQ ID NO: 343 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTGGGAAATGCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACGCATACCAGATGCATTGGGTGCGCCAGGCACCTGGTAAAGGTCTGGAATGGGTTGCCTTTATCTCTGATGATTCTGGTAACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACGATGTTTACGCATTTGATTACTGGGGTCAGGGCACC

SEQ ID NO: 344 - protein, 17 aa, synthetic construct
GDSSYSYHPYYSSAMDY

SEQ ID NO: 345 - protein, 206 aa, synthetic construct
KPGKAPKLLIYSASFLYSGVPSRFSGSRSGTDFTLTISSLQPEDFATYYCQQSGYSTPWTFGQGTKVEIKRTGGGSGGGGSGGGASEVQLVESGGGLVQPGGSLRLSCAASGFNLSQYKMHWVRQAPGKGLEWVAAIDGYSDNTNYADSVKGRFTISADTSKNTAYLQMNSLRAEDTAVYYCSRGDSSYSYHPYYSSAMDYWGQGT

SEQ ID NO: 346 - protein, 618 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTGGTTACTCTACCCCTTGGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTCTCAGTACAAAATGCATTGGGTGCGCCAGGCACCTGGTAAAGGTCTGGAATGGGTTGCCGCAATCGATGGTTACTCTGATAACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTGATTCTTCTTACTCTTACCATCCATACTACTCTTCTGCAATGGATTACTGGGGTCAGGGCACC

SEQ ID NO: 347 - protein, 12 aa, synthetic construct
NEMGSKLSGFDY

SEQ ID NO: 348 - protein, 200 aa, synthetic construct
KPGKAPKLLIYSASFLYSGVPSRFSGSRSGTDFTLTISSLQPEDFATYYCQQSYYYPITFGQGTKVEIKRTGGGSGGGGSGGGASEVQLVESGGGLVQPGGSLRLSCAASGFNISYDHMHWVRQAPGKGLEWVATLYPSGGFTNYADSVKGRFTISADTSKNTAYLQMNSLRAEDTAVYYCSRNEMGSKLSGFDYWGQGT

SEQ ID NO: 349 - protein, 600 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTACTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTACGATCATATGCATTGGGTGCGCCAGGCACCTGGTAAAGGTCTGGAATGGGTTGCCACCCTGTACCCATCTGGTGGTTTTACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACGAAATGGGTTCTAAACTGTCTGGTTTTGATTACTGGGGTCAGGGCACC

SEQ ID NO: 350 - protein, 12 aa, synthetic construct
NELCSKLSGFDY

SEQ ID NO: 351 - protein, 200 aa, synthetic construct
KPGKAPKLLIYSASFLYSGVPSRFSGSRSGTDFTLTISSLQPEDFATYYCQQSYYYPITFGQGTKVEIKRTGGGSGGGGSGGGASEVQLVESGGGLVQPGGSLRLSCAASGFNISYDHMHWVRQAPGKGLEWVATLYPSGGFTNYADSVKGRFTISADTSKNTAYLQMNSLRAEDTAVYYCSRNELCSKLSGFDYWGQGT

SEQ ID NO: 352 - protein, 600 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTTACTACTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTACGATCATATGCATTGGGTGCGCCAGGCACCTGGTAAAGGTCTGGAATGGGTTGCCACCCTGTACCCATCTGGTGGTTTTACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACGAACTGTGTTCTAAACTGTCTGGTTTTGATTACTGGGGTCAGGGCACC

SEQ ID NO: 353 - protein, 20 aa, synthetic construct
GQFYYSSYMGSYSYYNAFDY

SEQ ID NO: 354 - protein, 207 aa, synthetic construct
KPGKAPKLLIYSASFLYSGVPSRFSGSRSGTDFTLTISSLQPEDFATYYCQQQYSPITFGQGTKVEIKRTGGGSGGGGSGGGASEVQLVESGGGLVQPGGSLRLSCAASGFNIYSWQIHWVRQAPGKGLEWVAHLSYDGGNTYYADSVKGRFTISADTSKNTAYLQMNSLRAEDTAVYYCSRGQFYYSSYMGSYSYYNAFDYWGQGT

SEQ ID NO: 355 - protein, 621 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACAGTACTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTCTTGGCAGATCCATTGGGTGCGCCAGGCACCTGGTAAAGGTCTGGAATGGGTTGCCCATCTGTCTTACGATGGTGGTAACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCAGTTTTACTACTCTTCTTACATGGGTTCTTACTCTTACTACAACGCATTTGATTACTGGGGTCAGGGCACC

SEQ ID NO: 356 - protein, 9 aa, synthetic construct
AVYPYAMDV

SEQ ID NO: 357 - protein, 197 aa, synthetic construct
KPGKAPKLLIYSASFLYSGVPSRFSGSRSGTDFTLTISSLQPEDFATYYCQQSMSYPITFGQGTKVEIKRTGGGSGGGGSGGGASEVQLVESGGGLVQPGGSLRLSCAASGFNVYAYGMHWVRQAPGKGLEWVARFYDQDGYTYYADSVKGRFTISADTSKNTAYLQMNSLRAEDTAVYYCSRAVYPYAMDVWGQGT

SEQ ID NO: 358 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTATGTCTTACCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACGCATACGGTATGCATTGGGTGCGCCAGGCACCTGGTAAAGGTCTGGAATGGGTTGCCAGATTTTACGATCAGGATGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGTTTACCCATACGCAATGGATGTTTGGGGTCAGGGCACC

SEQ ID NO: 359 - protein, 9 aa, synthetic construct
NYSYYAFDV

SEQ ID NO: 360 - protein, 198 aa, synthetic construct
KPGKAPKLLIYSASFLYSGVPSRFSGSRSGTDFTLTISSLQPEDFATYYCQQVTVGSPTTFGQGTKVEIKRTGGGSGGGGSGGGASEVQLVESGGGLVQPGGSLRLSCAASGFNVYQYGMHWVRQAPGKGLEWVAKVSDQDGSTYYADSVKGRFTISADTSKNTAYLQMNSLRAEDTAVYYCSRNYSYYAFDVWGQGT

SEQ ID NO: 361 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGTTACCGTTGGTTCTCCTACCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACCAGTACGGTATGCATTGGGTGCGCCAGGCACCTGGTAAAGGTCTGGAATGGGTTGCCAAAGTTTCTGATCAGGATGGTTCTACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACTACTCTTACTACGCATTTGATGTTTGGGGTCAGGGCACC

SEQ ID NO: 362 - protein, 8 aa, synthetic construct
HLYMYFDY

SEQ ID NO: 363 - protein, 196 aa, synthetic construct
KPGKAPKLLIYSASFLYSGVPSRFSGSRSGTDFTLTISSLQPEDFATYYCQQMSSAPITFGQGTKVEIKRTGGGSGGGGSGGGASEVQLVESGGGLVQPGGSLRLSCAASGFNFMYYGMHWVRQAPGKGLEWVAHIFPGDSYTYYADSVKGRFTISADTSKNTAYLQMNSLRAEDTAVYYCSRHLYMYFDYWGQGT

SEQ ID NO: 364 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAATGTCTTCTGCACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACTTTATGTACTACGGTATGCATTGGGTGCGCCAGGCACCTGGTAAAGGTCTGGAATGGGTTGCCCATATCTTTCCAGGTGATTCTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATCTGTACATGTACTTTGATTACTGGGGTCAGGGCACC

SEQ ID NO: 365 - protein, 18 aa, synthetic construct
ADYYYSHSYYYSSSYLDV

SEQ ID NO: 366 - protein, 206 aa, synthetic construct
KPGKAPKLLIYSASFLYSGVPSRFSGSRSGTDFTLTISSLQPEDFATYYCQQAGYYPVTFGQGTKVEIKRTGGGSGGGGSGGGASEVQLVESGGGLVQPGGSLRLSCAASGFNIYSYDMHWVRQAPGKGLEWVAAISPDDDYTYYADSVKGRFTISADTSKNTAYLQMNSLRAEDTAVYYCSRADYYYSHSYYYSSSYLDVWGQGT

SEQ ID NO: 367 - protein, 618 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGCAGGTTACTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTCTTACGATATGCATTGGGTGCGCCAGGCACCTGGTAAAGGTCTGGAATGGGTTGCCGCAATCTCTCCAGATGATGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGATTACTACTACTCTCACTCTTACTACTACTCTTCTTCTTACCTGGATGTTTGGGGTCAGGGCACC

SEQ ID NO: 368 - protein, 10 aa, synthetic construct
YSSQSYYMDY

SEQ ID NO: 369 - protein, 198 aa, synthetic construct
KPGKAPKLLIYSASFLYSGVPSRFSGSRSGTDFTLTISSLQPEDFATYYCQQQYDTPITFGQGTKVEIKRTGGGSGGGGSGGGASEVQLVESGGGLVQPGGSLRLSCAASGFNISSYGIHWVRQAPGKGLEWVAFFYPDSSYTYYADSVKGRFTISADTSKNTAYLQMNSLRAEDTAVYYCSRYSSQSYYMDYWGQGT

SEQ ID NO: 370 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAACAGTACGATACCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTTCTTACGGTATCCATTGGGTGCGCCAGGCACCTGGTAAAGGTCTGGAATGGGTTGCCTTTTTTTACCCAGATTCTTCTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTACTCTTCTCAGTCTTACTACATGGATTACTGGGGTCAGGGCACC

SEQ ID NO: 371 - protein, 9 aa, synthetic construct
ELYEYAFDY

SEQ ID NO: 372 - protein, 196 aa, synthetic construct
KPGKAPKLLIYSASFLYSGVPSRFSGSRSGTDFTLTISSLQPEDFATYYCQQYSYPVTFGQGTKVEIKRTGGGSGGGGSGGGASEVQLVESGGGLVQPGGSLRLSCAASGFNVTASNMHWVRQAPGKGLEWVAIVSPDSGYTYYADSVKGRFTISADTSKNTAYLQMNSLRAEDTAVYYCSRELYEYAFDYWGQGT

SEQ ID NO: 373 - protein, 588 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTCTTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTACCGCATCTAACATGCATTGGGTGCGCCAGGCACCTGGTAAAGGTCTGGAATGGGTTGCCATCGTTTCTCCAGATTCTGGTTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAACTGTACGAATACGCATTTGATTACTGGGGTCAGGGCACC

SEQ ID NO: 374 - protein, 9 aa, synthetic construct
SYGNYAFDY

SEQ ID NO: 375 - protein, 198 aa, synthetic construct
KPGKAPKLLIYSASFLYSGVPSRFSGSRSGTDFTLTISSLQPEDFATYYCQQTWSSSPVTFGQGTKVEIKRTGGGSGGGGSGGGASEVQLVESGGGLVQPGGSLRLSCAASGFNIYQYVIHWVRQAPGKGLEWVALVTPDDGYTNYADSVKGRFTISADTSKNTAYLQMNSLRAEDTAVYYCSRSYGNYAFDYWGQGT

SEQ ID NO: 376 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAACCTGGTCTTCTTCTCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACCAGTACGTTATCCATTGGGTGCGCCAGGCACCTGGTAAAGGTCTGGAATGGGTTGCCCTGGTTACCCCAGATGATGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACGGTAACTACGCATTTGATTACTGGGGTCAGGGCACC

SEQ ID NO: 377 - protein, 18 aa, synthetic construct
ADYYYSNSYYYSSSYLDV

SEQ ID NO: 378 - protein, 206 aa, synthetic construct
KPGKAPKLLIYSASFLYSGVPSRFSGSRSGTDFTLTISSLQPEDFATYYCQQAGYYPVTFGQGTKVEIKRTGGGSGGGGSGGGASEVQLVESGGGLVQPGGSLRLSCAASGFNIYSYDMHWVRQAPGKGLEWVAAISPDDDYTYYADSVKGRFTISADTSKNTAYLQMNSLRAEDTAVYYCSRADYYYSNSYYYSSSYLDVWGQGT

SEQ ID NO: 379 - protein, 618 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGCAGGTTACTACCCTGTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTACTCTTACGATATGCATTGGGTGCGCCAGGCACCTGGTAAAGGTCTGGAATGGGTTGCCGCAATCTCTCCAGATGATGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGCAGATTACTACTACTCTAACTCTTACTACTACTCTTCTTCTTACCTGGATGTTTGGGGTCAGGGCACC

SEQ ID NO: 380 - protein, 9 aa, synthetic construct
SYMTYAFDY

SEQ ID NO: 381 - protein, 197 aa, synthetic construct
KPGKAPKLLIYSASFLYSGVPSRFSGSRSGTDFTLTISSLQPEDFATYYCQQSTYKPITFGQGTKVEIKRTGGGSGGGGSGGGASEVQLVESGGGLVQPGGSLRLSCAASGFNLYSYSIHWVRQAPGKGLEWVAAINSDDGYTNYADSVKGRFTISADTSKNTAYLQMNSLRAEDTAVYYCSRSYMTYAFDYWGQGT

SEQ ID NO: 382 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATCTACCTACAAACCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACCTGTACTCTTACTCTATCCATTGGGTGCGCCAGGCACCTGGTAAAGGTCTGGAATGGGTTGCCGCGATCAACTCTGATGATGGTTACACCAACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCTCTTACATGACCTACGCATTTGATTACTGGGGTCAGGGCACC

SEQ ID NO: 383 - protein, 11 aa, synthetic construct
ETSYAFSAMDY

SEQ ID NO: 384 - protein, 199 aa, synthetic construct
KPGKAPKLLIYSASFLYSGVPSRFSGSRSGTDFTLTISSLQPEDFATYYCQQYYYSPITFGQGTKVEIKRTGGGSGGGGSGGGASEVQLVESGGGLVQPGGSLRLSCAASGFNVSYGDMHWVRQAPGKGLEWVAKFHDGSGHTYYADSVKGRFTISADTSKNTAYLQMNSLRAEDTAVYYCSRETSYAFSAMDYWGQGT

SEQ ID NO: 385 - protein, 597 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACTACTACTCTCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTCTTACGGTGATATGCATTGGGTGCGCCAGGCACCTGGTAAAGGTCTGGAATGGGTTGCCAAATTTCATGATGGTTCTGGTCACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGAAACCTCTTACGCATTTTCTGCAATGGATTACTGGGGTCAGGGCACC

SEQ ID NO: 386 - protein, 10 aa, synthetic construct
HHGGYYALDY

SEQ ID NO: 387 - protein, 198 aa, synthetic construct
KPGKAPKLLIYSASFLYSGVPSRFSGSRSGTDFTLTISSLQPEDFATYYCQQFSYSPFTFGQGTKVEIKRTGGGSGGGGSGGGASEVQLVESGGGLVQPGGSLRLSCAASGFNISGSSMHWVRQAPGKGLEWVAQIYSGYDYTYYADSVKGRFTISADTSKNTAYLQMNSLRAEDTAVYYCSRHHGGYYALDYWGQGT

SEQ ID NO: 388 - protein, 594 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATTTTCTTACTCTCCTTTTACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACATCTCTGGTTCTTCTATGCATTGGGTGCGCCAGGCACCTGGTAAAGGTCTGGAATGGGTTGCCCAGATCTACTCTGGTTACGATTACACCTACTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCCATCATGGTGGTTACTACGCACTGGATTACTGGGGTCAGGGCACC

SEQ ID NO: 389 - protein, 14 aa, synthetic construct
GQDSHATYYYSFDV

SEQ ID NO: 390 - protein, 202 aa, synthetic construct
KPGKAPKLLIYSASFLYSGVPSRFSGSRSGTDFTLTISSLQPEDFATYYCQQDHYYPMTFGQGTKVEIKRTGGGSGGGGSGGGASEVQLVESGGGLVQPGGSLRLSCAASGFNVGNSGMHWVRQAPGKGLEWVAYVYDGSDSTEYADSVKGRFTISADTSKNTAYLQMNSLRAEDTAVYYCSRGQDSHATYYYSFDVWGQGT

SEQ ID NO: 391 - protein, 606 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAAGATCATTACTACCCTATGACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTGGTAACTCTGGTATGCATTGGGTGCGCCAGGCACCTGGTAAAGGTCTGGAATGGGTTGCCTACGTTTACGATGGTTCTGATTCTACCGAATATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCGGTCAGGATTCTCATGCAACCTACTACTACTCTTTTGATGTTTGGGGTCAGGGCACC

SEQ ID NO: 392 - protein, 9 aa, synthetic construct
NYSKYAMDY

SEQ ID NO: 393 - protein, 197 aa, synthetic construct
KPGKAPKLLIYSASFLYSGVPSRFSGSRSGTDFTLTISSLQPEDFATYYCQQYNYTPITFGQGTKVEIKRTGGGSGGGGSGGGASEVQLVESGGGLVQPGGSLRLSCAASGFNVYAYSMHWVRQAPGKGLEWVATFSDDSGVTGYADSVKGRFTISADTSKNTAYLQMNSLRAEDTAVYYCSRNYSKYAMDYWGQGT

SEQ ID NO: 394 - protein, 591 aa, synthetic construct
AAACCGGGTAAAGCACCGAAACTGCTGATTTATAGCGCCTCTTTTCTGTATAGCGGTGTGCCGAGCCGTTTTAGCGGTAGCCGTAGCGGCACCGATTTTACCCTGACCATTAGCAGCCTGCAGCCGGAAGATTTTGCAACCTATTATTGCCAGCAATACAACTACACCCCTATCACCTTTGGCCAGGGCACCAAAGTTGAAATTAAACGTACCGGTGGTGGTTCAGGTGGCGGTGGCAGCGGAGGCGGAGCCAGCGAAGTGCAGCTGGTTGAAAGCGGTGGCGGTCTGGTTCAGCCAGGTGGTAGCCTGCGTCTGAGCTGTGCAGCAAGCGGTTTTAACGTTTACGCATACTCTATGCATTGGGTGCGCCAGGCACCTGGTAAAGGTCTGGAATGGGTTGCCACCTTTTCTGATGATTCTGGTGTTACCGGTTATGCCGATAGCGTGAAAGGTCGCTTTACCATTAGCGCAGATACCAGCAAAAATACCGCATACCTGCAGATGAATAGTCTGCGTGCAGAGGATACAGCAGTGTATTATTGTAGCCGCAACTACTCTAAATACGCAATGGATTACTGGGGTCAGGGCACC

SEQ ID NO: 395 - DNA, 22 bp, synthetic construct
  source 1..22: mol_type = other DNA
atgaaatacctattgcctacgg

SEQ ID NO: 396 - DNA, 28 bp, synthetic construct
  source 1..28: mol_type = other DNA
cgttagtaaatgaattttctgtatgagg